(19) United States
(12) Patent Application Publication
(10) Pub. No.: US 2024/0083999 A1
(43) Pub. Date: Mar. 14, 2024

(54) VHH POLYPEPTIDES THAT BIND TO INTERLEUKIN 6 (IL-6), COMPOSITIONS AND METHODS OF USE THEREOF

(71) Applicants: Trustees of Tufts College, Medford, MA (US); Vicero, Inc., Shrewsbury, MA (US)

(72) Inventors: Charles B. SHOEMAKER, Medford, MA (US); Vikram KANSRA, Shrewsbury, MA (US)

(73) Assignees: Trustees of Tufts College, Medford, MA (US); Vicero, Inc., Shrewsbury, MA (US)

(21) Appl. No.: 18/501,648

(22) Filed: Nov. 3, 2023

Related U.S. Application Data
(63) Continuation of application No. PCT/US2022/027439, filed on May 3, 2022.
(60) Provisional application No. 63/184,441, filed on May 5, 2021.

(51) Int. Cl. C07K 16/24; C12N 15/85

(52) U.S. Cl. CPC C07K 16/248; C12N 15/85; A61K 2039/505; C07K 2317/22; C07K 2317/569; C07K 2317/626

(57) ABSTRACT

Described herein are single domain VHH polypeptides (antibodies) that bind to and/or neutralize the cytokine interleukin 6 (IL-6), in particular, human IL-6 (hIL-6). Anti-hIL-6 VHH polypeptide products, methods, pharmaceutical compositions, and kits are provided for treating a subject having or at risk of having a disease, disorder, pathology, or infection associated with or induced by IL-6 or dysfunctional IL-6 signaling. The methods, compositions and kits include a single domain, anti-hIL-6 VHH polypeptide (antibody), or hIL-6 binding portion thereof, that specifically binds to and/or neutralizes IL-6 and treats or prevents IL-6-induced illnesses and diseases. The anti-hIL-6 VHHs, or hIL-6 binding portion thereof, may be recombinantly produced and expressed.

### CROSS REFERENCE TO RELATED APPLICATIONS

This application is a continuation under 35 U.S.C. § 111(a) of PCT International Patent Application No. PCT/US2022/027439, filed May 3, 2022, designating the United States and published in English, which claims priority to and the benefit of U.S. Provisional Application No. 63/184,441, filed May 5, 2021, the entire contents of each of which are incorporated by reference herein.

### SEQUENCE LISTING

The present application contains a Sequence Listing which has been submitted electronically in XML format and is herein incorporated by reference in its entirety. The Sequence Listing XML file, created on Nov. 3, 2023, is named 167774-012602US-Sequence_Listing.xml and is 170,174 bytes in size.

### BACKGROUND

Interleukin 6 (IL-6) is an endogenous single chain glycoprotein and cytokine, which is produced by several different cell types and is active in both acute and chronic inflammation and other diseases and pathologies. The IL-6 protein binds to the IL-6 receptor (IL-6R) on the surface of cells to induce transcription of inflammatory gene products. In addition, IL-6 can bind to soluble IL-6R; thereafter, an IL-6/IL-6R complex may directly activate cells. IL-6 promotes B-cell maturation and T-cell differentiation, while concurrently synergizing with tumor necrosis factor-alpha (TNF-α) and interleukin 1 glycoprotein (IL-1) to promote a systemic inflammatory response. IL-6 can also function as a pyrogen, causing fever in autoimmune, infectious diseases and non-infectious diseases. Produced in the body wherever there is inflammation, IL-6 is involved in numerous conditions, diseases and disorders, such as trauma, burns, cancers and infection.

Cytokine storm (CS) has been attributed as the major cause of morbidity, multi-organ failure and mortality in patients having a number of diseases, for example, inflammatory diseases, autoimmune diseases, cancer and infectious diseases, including viral infection. The use of IL-6R inhibitors or other IL-6 blocking agents have shown only modest benefit in the treatment of diseases, especially in acutely ill patients, due to a need for high doses of such agents, as well as their associated side effects. In addition, the cost of treatment using such agents may be prohibitive.

Thus, there is a profound need for new, efficacious, and cost effective therapeutic agents that target IL-6 and safely treat subjects afflicted with conditions, diseases, disorder, infections, and the symptoms thereof, involving or associated with IL-6 and its activity. The present invention provides a solution to such a need.

### SUMMARY

Described herein are VHH-based polypeptides (antibodies) that specifically bind to interleukin-6 (IL-6) produced by various cells in a mammalian subject, including, without limitation, monocytes, macrophages, dendritic cells, endothelial cells and cells of the immune system. In an embodiment, the interleukin-6 is human interleukin-6. The VHH polypeptides described herein are monomeric or multimeric, e.g., dimeric, single chain antibodies that specifically bind to human IL-6 protein (anti-hIL-6 VHHs). In some embodiments, the anti-hIL-6 VHHs both bind to and neutralize human IL-6 (hIL-6). In some embodiments, the anti-hIL-6 VHHs as described herein bind to and neutralize human IL-6 in vitro and/or in vivo. As would be appreciated by the skilled practitioner in the art, anti-hIL-6 VHH antibodies are also synonymously known as “single domain antibodies (sdAbs)” or “nanobodies (NBs).”

In an aspect, the present invention provides a VH-heavy chain only (VHH) binding protein or an antigen binding portion thereof that specifically binds to interleukin-6 (IL-6), wherein the binding protein or the antigen binding portion thereof comprises three Complementarity Determining Regions (CDRs), CDR1, CDR2 and CDR3, which are structurally positioned between four camelid VHH framework (FR) regions (FR1-FR4) as follows: FR1-CDR1-FR2-CDR2-FR3-CDR3-FR4; wherein the three CDRs are selected from:
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13);
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13);
CDR1 comprising amino acid sequence GFTLDYYA (SEQ ID NO: 11); CDR2 comprising amino acid sequence SSSDRSAY (SEQ ID NO: 14); and CDR3 comprising amino acid sequence GTWDLKWGYNISACVRSYEYDY (SEQ ID NO: 15);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFALDYYA (SEQ ID NO: 18); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYG (SEQ ID NO: 19); CDR2 comprising amino acid sequence SSSDLKTY (SEQ ID NO: 16); and CDR3 comprising amino acid sequence GTWDLKFGYNITTCVRSSEYDY (SEQ ID NO: 20);
CDR1 comprising amino acid sequence GFTSDYYG (SEQ ID NO: 21); CDR2 comprising amino acid sequence SSSDLKTY (SEQ ID NO: 16); and CDR3 comprising amino acid sequence GTWDLKFGYNITTCVRSSEYDY (SEQ ID NO: 20);
CDR1 comprising amino acid sequence GFTLDYYG (SEQ ID NO: 19); CDR2 comprising amino acid sequence SSSDWSTY (SEQ ID NO: 22); and CDR3 comprising amino acid sequence GTWDLKFGYNRSNCVRSAEYDY (SEQ ID NO: 23); or
CDR1 comprising amino acid sequence GFTLAYYG (SEQ ID NO: 24); CDR2 comprising amino acid sequence SSSDLSTY (SEQ ID NO: 25); and CDR3 comprising amino acid sequence GTWDLKFGYSRSNCVRSYEYDY (SEQ ID NO: 26). In an embodiment, in the VHH binding protein, FR1 comprises 20 consecutive amino acids comprising X₁-X₂-G-G-G-L-V-Q-P-G-G-S-X₃-X₄-L-S-C-A-A-S(SEQ ID NO: 27), wherein X₁ is absent or T; X₂ is S, T, or G; X₃ is L or Q; and X₄ is R or G; FR2 comprises 18 consecutive amino acids comprising X₁-G-W-F-R-Q-A-P-G-K-E-R-E-G-X₂-X₃-C-X₄ (SEQ ID NO: 28), wherein X₁ is I or V; X₂ is V or I; X₃ is S or A; and X₄ is L, I, or M; FR3 comprises 38 consecutive amino acids comprising X₁-D-S-V-K-G-R-F-T-I-S-R-D-X₂-X₃-X₄-X₅-X₆-X₇-X₈-L-Q-M-N-S-L-K-P-E-D-T-X₉-X₁₀-Y-Y-C-A-A (SEQ ID NO: 29), wherein X₁ is V, I, T, or A; X₂ is D, G, N, Y, or S; X₃ is D or A; X₄ is K or N; X₅ is N, S, or D; X₆ is T or A; X₇ is A or V; X₈ is Y or S; X₉ is A or G; and X₁₀ is T or V; and FR4 comprises 11 consecutive amino acids comprising X₁-X₂-Q-G T Q V T V S S (SEQ ID NO: 30), wherein X₁ is W or R; and X₂ is G or D.

In an aspect, the present invention provides a VH-heavy chain only (VHH) binding protein or an antigen binding portion thereof that specifically binds to interleukin-6 (IL-6), wherein the binding protein or the antigen binding portion thereof comprises three Complementarity Determining Regions (CDRs), CDR1, CDR2 and CDR3, which are structurally positioned between four camelid VHH framework (FR) regions (FR1-FR4) as follows: FR1-CDR1-FR2-CDR2-FR3-CDR3-FR4; wherein the three CDRs are selected from:
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GRPFSSFA (SEQ ID NO: 31); CDR2 comprising amino acid sequence TWSRGTTH (SEQ ID NO: 32); and CDR3 comprising amino acid sequence AAADGWKVVSTASPAYDY (SEQ ID NO: 33);
CDR1 comprising amino acid sequence GFTLAYYG (SEQ ID NO: 24); CDR2 comprising amino acid sequence SSSDLSTY (SEQ ID NO: 25); and CDR3 comprising amino acid sequence GTWDLKFGYSRSNCVRSYEYDY (SEQ ID NO: 26);
CDR1 comprising amino acid sequence GRTFSSRA (SEQ ID NO: 34); CDR2 comprising amino acid sequence SWTGSPY (SEQ ID NO: 35); and CDR3 comprising amino acid sequence AATSEHVMLVVTTRGGYDY (SEQ ID NO: 36); or
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13). In an embodiment, in the VHH binding protein, FR1 comprises 25 consecutive amino acids comprising Q-X₁-Q-L-X₂-E-X₃-G-G-G-X₄-V-Q-X₅-G-X₆-S-L-X₇-L-S-C-A-A-S(SEQ ID NO: 37), wherein X₁ is L or V; X₂ is A or V; X₃ is T or S; X₄ is L or S; X₅ is P or A; X₆ is G or D; X₇ is R, T or G; FR2 comprises 18 consecutive amino acids comprising X₁-G-W-F-R-Q-A-P-G-K-E-R-E-X₂-X₃-X₄-X₅-X₆ (SEQ ID NO: 38), wherein X₁ is V, M, or I; X₂ is F or G; X₃ is I or V; X₄ is S or A; X₅ is C, A or V; X₆ is I or L; FR3 comprises 37-39 consecutive amino acids comprising Y-X₁-D-S-V-K-G-R-F-T-I-S-X₂-D-X₃-X₄-K-X₅-T-X₆-X₇-L-Q-M-N-S-L-K-P-E-D-T-X₈-X₉-Y-Y-C-A-A (SEQ ID NO: 39), wherein X₁ is A, T, or V; X₂ is R or G; X₃ is Y, N, or D; X₄ is A or D; X₅ is S, N, or D; X₆ is V or A; X₇ is S, F, or Y; X₈ is G or A; X₉ is V or T; and FR4 comprises 11 consecutive amino acids comprising W-X₁-Q-G-T-Q-V-T-V-S-S(SEQ ID NO: 40), wherein X₁ is D or G.

In an embodiment of the above aspects and/or the embodiments thereof, the VHH binding protein neutralizes interleukin-6 (IL-6) or activity thereof. In an embodiment, the VHH binding protein is a camelid-derived single domain anti-hIL-6 VHH antibody. In an embodiment, the VHH binding protein is recombinantly produced. In an embodiment, the VHH binding protein is in the form of a dimer or multimer. In an embodiment, the VHH binding protein is in the form of a homodimer. In embodiment, the dimer, homodimer, or multimer comprises the VHH binding proteins separated by a spacer or linker. In an embodiment, the VHH binding protein includes one or more epitope tag sequences specifically bindable by an anti-epitope tag antibody or binding portion thereof. In an embodiment, the one or more epitope tag sequences comprises at least one of DELGPRLMGK (SEQ ID NO: 41) or GAPVPYPDPLEPR (SEQ ID NO: 42). In an embodiment, the VHH binding protein neutralizes hIL-6 activity in vitro or in vivo. In an embodiment, the VHH binding protein reduces or abolishes JAK-STAT signaling in vitro or in vivo.

In an aspect of the invention, a polypeptide that specifically binds to human interleukin-6 (hTL-6) is provided, in which the polypeptide or a hIL-6-binding portion thereof has at least 85% amino acid sequence identity to a sequence selected from the group consisting of SEQ ID NOs: 1, 3, 5, 7 and 9.

In an aspect of the invention, a polypeptide that specifically binds to human interleukin-6 (hTL-6) is provided, in which the polypeptide or a hIL-6-binding portion thereof has at least 90% amino acid sequence identity to a sequence selected from the group consisting of SEQ ID NOs: 1, 3, 5, 7 and 9.

In an aspect of the invention, a polypeptide that specifically binds to human interleukin-6 (hTL-6) is provided, in which the polypeptide or a hIL-6-binding portion thereof has at least 95% amino acid sequence identity to a sequence selected from the group consisting of SEQ ID NOs: 1, 3, 5, 7 and 9.

In an embodiment of the foregoing aspects, conservative amino acid substitutions in the polypeptide comprise the at least 85%, at least 90%, at least 95%, or at least 98% amino acid sequence identity.

In an aspect of the invention, a polypeptide that specifically binds to human interleukin-6 (hTL-6) is provided, in which the polypeptide or a hIL-6-binding portion thereof comprises a sequence selected from the group consisting of SEQ ID NOs: 1, 3, 5, 7 and 9.

In an aspect of the invention, a polypeptide that specifically binds to human interleukin-6 (hTL-6) is provided, in which the polypeptide or a hIL-6-binding portion thereof consists of a sequence selected from the group consisting of SEQ ID NOs: 1, 3, 5, 7 and 9.

In an embodiment of the above-delineated aspects, the polypeptide neutralizes hTL-6 activity in vitro or in vivo. In an embodiment, the polypeptide reduces or abolishes JAK-STAT signaling in vitro or in vivo. In an embodiment, the polypeptide is a camelid-derived single domain VHH antibody (VHH). In an embodiment, the polypeptide is in the form of a dimer or multimer. In an embodiment, the polypeptide is in the form of a homodimer. In an embodiment, the dimer, homodimer, or multimer comprises the hIL-6 binding polypeptides separated by a spacer or linker. In an embodiment, the polypeptide includes one or more epitope tag sequences specifically bindable by an anti-epitope tag antibody or binding portion thereof. In an embodiment, the one or more epitope tag sequences comprises at least one of DELGPRLMGK (SEQ ID NO: 41) or GAPVPYPDPLEPR (SEQ ID NO: 42). In an embodiment of the above-delineated aspects of the binding protein or polypeptide and/or embodiments thereof, the binding protein or polypeptide is linked to an immunoglobulin Fc domain.

In another aspect of the invention, a dimeric or multimeric polypeptide comprising two or more anti-hIL-6 VHH polypeptides comprising a sequence selected from the group consisting of SEQ ID NOs: 1, 3, 5, 7, and 9, or hIL-6-binding regions thereof is provided, wherein the two or more anti-hIL-6 VHH polypeptides, or hIL-6-binding regions, are joined with one or more spacer or linker peptides. In an embodiment of the dimeric or multimeric polypeptide, the one or more linker peptides is selected from GGGGS (SEQ ID NO: 43); GGGGSGGGGSGGGGS (GGGGS)₃ (SEQ ID NO: 44), or a functional portion thereof, EPKTPKPQGGGGSGGGGSGGGGSQGVQSQVQLVE (SEQ ID NO: 45); EPKTPKPQ (SEQ ID NO: 46); or a combination thereof. In an embodiment, the dimeric or multimeric polypeptide includes one or more epitope tag sequences specifically bindable by an anti-epitope tag antibody or binding portion thereof. In an embodiment, the one or more epitope tag sequences comprises at least one of DELGPRLMGK (SEQ ID NO: 41) or GAPVPYPDPLEPR (SEQ ID NO: 42). In an embodiment, the dimeric or multimeric polypeptide is dimeric and comprises two anti-hIL-6 VHH polypeptides. In an embodiment of the dimeric polypeptide, the two anti-hIL-6 VHH polypeptides are the same and in the form of a homodimer. In an embodiment, the homodimer comprises two anti-hIL-6 VHH polypeptides of SEQ ID NO: 5 joined with one or more spacer or linker peptides. In an embodiment of the dimeric polypeptide, the two anti-hTL-6 VHH polypeptides are different. In an embodiment of the dimeric or multimeric polypeptide, the polypeptide is multimeric and comprises at least three anti-hTL-6 VHH polypeptides. In an embodiment of the dimeric or multimeric polypeptide, the polypeptide is multimeric and comprises at least four anti-hIL-6 VHH polypeptides. In an embodiment, the multimeric polypeptide comprises three or four anti-hTL-6 VHH polypeptides. In an embodiment of the multimeric polypeptide, the anti-hIL-6 VHH polypeptides are the same or different. In an embodiment of the multimeric polypeptide, the anti-hIL-6 VHH polypeptides are a combination of the same and different anti-hIL-6 VHH polypeptides. In an embodiment, the dimeric or multimeric polypeptide is linked to an immunoglobulin Fc domain.

In another aspect of the present invention, an isolated polynucleotide encoding the binding protein or polypeptide of any of the above-delineated aspects and/or embodiments thereof is provided.

In another aspect of the present invention, an isolated polynucleotide encoding the dimeric or multimeric polypeptide of any of the above-delineated aspects and/or embodiments thereof is provided.

In another aspect of the present invention, an isolated polynucleotide having at least 90% sequence identity to a nucleic acid sequence selected from the group consisting of SEQ ID NOs: 2, 4, 6, 8 and 10 is provided.

In another aspect of the present invention, an isolated polynucleotide having at least 95% sequence identity to a nucleic acid sequence selected from the group consisting of SEQ ID NOs: 2, 4, 6, 8 and 10 is provided.

In another aspect of the present invention, an isolated polynucleotide having at least 98% sequence identity to a nucleic acid sequence selected from the group consisting of SEQ ID NOs: 2, 4, 6, 8 and 10 is provided.

In another aspect of the present invention, an isolated polynucleotide comprising a sequence selected from the group consisting of SEQ ID NOs: 2, 4, 6, 8 and 10 is provided.

In another aspect of the present invention, an isolated polynucleotide consisting of a sequence selected from the group consisting of SEQ ID NOs: 2, 4, 6, 8 and 10 is provided.

In another aspect of the present invention, an isolated polynucleotide comprising a nucleic acid sequence encoding an anti-hIL-6 VHH of any one of SEQ ID NOs: 1, 3, 5, 7, or 9 is provided.

In another aspect of the present invention, an isolated polynucleotide having at least 85% sequence identity to a nucleic acid sequence encoding an anti-hIL-6 VHH of any one of SEQ ID NOs: 1, 3, 5, 7, or 9 is provided.

In another aspect of the present invention, an isolated polynucleotide having at least 90% sequence identity to a nucleic acid sequence encoding an anti-hIL-6 VHH of any one of SEQ ID NOs: 1, 3, 5, 7, or 9 is provided.

In another aspect of the present invention, an isolated polynucleotide having at least 95% sequence identity to a nucleic acid sequence encoding an anti-hIL-6 VHH of any one of SEQ ID NOs: 1, 3, 5, 7, or 9 is provided.

In another aspect of the present invention, an isolated polynucleotide having at least 98% sequence identity to a nucleic acid sequence encoding an anti-hIL-6 VHH of any one of SEQ ID NOs: 1, 3, 5, 7, or 9 is provided.

In an embodiment of the isolated polynucleotide of any of the above-delineated aspects and/or embodiments thereof, the polynucleotide is DNA or RNA. In an embodiment, the polynucleotide is mRNA.

In another aspect of the present invention, a vector comprising a nucleic acid molecule that encodes the binding protein or polypeptide of any of the above-delineated aspects and/or embodiments thereof is provided.

In another aspect of the present invention, a vector comprising a nucleic acid molecule that encodes the dimeric or multimeric polypeptide of any of the above-delineated aspects and/or embodiments thereof is provided.

In another aspect of the present invention, a vector comprising the isolated polynucleotide of any of the above-delineated aspects and/or embodiments thereof is provided.

In an embodiment of the vector of any of the above-delineated aspects and/or embodiments thereof, the vector is an expression vector. In an embodiment, the expression vector is a viral or non-viral expression vector.

In another aspect of the present invention, a host cell comprising the vector of any of the above-delineated aspects and embodiments thereof is provided.

In another aspect of the present invention, a pharmaceutical composition comprising an effective amount of the binding protein or the polypeptide of any of the above-delineated aspects and/or embodiments thereof, or a hTL-6 binding fragment thereof, and a pharmaceutically acceptable excipient, carrier, or diluent is provided.

In another aspect of the present invention, a pharmaceutical composition comprising an effective amount of the dimeric or multimeric polypeptide of any of the above-delineated aspects and/or embodiments thereof, or a hTL-6 binding fragment thereof, and a pharmaceutically acceptable excipient, carrier, or diluent is provided.

In another aspect of the present invention, a pharmaceutical composition comprising an effective amount of the isolated polynucleotide of any of the above-delineated aspects and/or embodiments thereof, and a pharmaceutically acceptable excipient, carrier, or diluent is provided.

In another aspect of the present invention, a method of neutralizing interleukin-6 (IL-6) activity is provided, in which the method involves contacting a cell with an effective amount of the binding protein or the polypeptide of any of the above-delineated aspects and/or embodiments thereof, thereby neutralizing IL-6 activity.

In another aspect of the present invention, a method of neutralizing interleukin-6 (IL-6) activity is provided, in which the method involves contacting a cell with an effective amount of the isolated polynucleotide of any of the above-delineated aspects and/or embodiments thereof, thereby neutralizing IL-6 activity.

In another aspect of the present invention, a method of inhibiting interleukin-6 (IL-6)-induced STAT3 activation is provided, in which the method involves contacting a cell with an effective amount of the binding protein or the polypeptide of any of the above-delineated aspects and/or embodiments thereof, thereby inhibiting IL-6-induced STAT3 activation.

In another aspect of the present invention, a method of inhibiting interleukin-6 (IL-6)-induced STAT3 activation is provided, in which the method involves contacting a cell with an effective amount of the isolated polynucleotide of any of the above-delineated aspects and/or embodiments thereof, thereby inhibiting IL-6-induced STAT3 activation.

In embodiments of the above-delineated methods and/or embodiments thereof, the cell is in vitro, ex vivo, or in vivo. In embodiments of the method, the cell is an hepatocyte, an endothelial cell, a monocyte, a macrophage, a T cell, a B cell, a fibroblast, a keratinocyte, or an adipocyte.

In another aspect of the present invention, a method of treating an interleukin-6 (IL-6)-mediated disease, disorder, pathology or infection and/or the symptoms thereof in a subject is provided, in which the method involves administering to a subject in need thereof an effective amount of the pharmaceutical composition of any of the above-delineated aspects and/or embodiments thereof, thereby treating the IL-6-mediated disease, disorder, pathology or infection and/or the symptoms thereof in the subject.

In another aspect of the present invention, a method of ameliorating, abrogating, or treating cytokine storm associated with an interleukin-6 (IL-6)-mediated disease, disorder, pathology or infection and/or the symptoms thereof in a subject is provided, in which the method involves administering to a subject in need thereof an effective amount of the pharmaceutical composition of any of the above-delineated aspects and/or embodiments thereof, thereby ameliorating, abating, or treating cytokine storm and/or the symptoms thereof in the subject.

In embodiments of the above methods, the IL-6-mediated disease, disorder, pathology or infection and/or the symptoms is associated with or caused by excess amounts, levels, or production of IL-6, or with dysregulation of IL-6 and/or IL-6 signaling. In embodiments of the above methods, the IL-6-mediated disease, disorder, pathology or infection is a viral or bacterial infections, a cancer, a carcinoma, a tumor, a cholangiocarcinoma, ovarian cancer, multiple myeloma; an autoimmune disease, an inflammatory disease, adult rheumatoid arthritis, juvenile idiopathic arthritis, Castleman's disease, secondary amyloidosis, polymyalgia rheumatic, adult onset Still's disease, polymyositis, systemic sclerosis, large vessel vasculitis lupus erythematosus, Crohn's disease, irritable bowel disease (IBD), Sjogren's syndrome; steroid refractory Graft versus Host Disease in transplantation; type 2 diabetes, obesity, or schizophrenia. In an embodiment of the above methods, the IL-6-mediated disease, disorder, pathology or infection is a viral infection. In an embodiment, the viral infection is Covid-19 infection or Adult Respiratory Distress Syndrome (ARDS). In an embodiment of the above methods, the subject is a mammal. In an embodiment of the above methods, the subject is a human. In an embodiment of the above methods, method reduces the severity of the IL-6-mediated disease, disorder, pathology or infection. In an embodiment of the above methods, the method further involves administering to the subject an anti-epitope tag antibody that specifically binds to an epitope tag, if present, and facilitates clearance of a complex of hIL-6 bound to the anti-hIL-6 VHH polypeptide from the subject.

In another aspect of the present invention, a polypeptide that specifically binds to and neutralizes human interleukin-6 (hIL-6), or a hIL-6-binding portion thereof is provided, wherein the polypeptide comprises three complementarity determining regions (CDRs), CDR1, CDR2 and CDR3, and four camelid VHH framework regions (FRs), FR1, FR2, FR3 and FR4, wherein the FRs structurally and positionally support CDR1-CDR3 therebetween as follows: FR1-CDR1-FR2-CDR2-FR3-CDR3-FR4, and wherein the three CDRs are selected from:
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GRPFSSFA (SEQ ID NO: 31); CDR2 comprising amino acid sequence TWSRGTTH (SEQ ID NO: 32); and CDR3 comprising amino acid sequence AAADGWKVVSTASPAYDY (SEQ ID NO: 33);
CDR1 comprising amino acid sequence GFTLAYYG (SEQ ID NO: 24); CDR2 comprising amino acid sequence SSSDLSTY (SEQ ID NO: 25); and CDR3 comprising amino acid sequence GTWDLKFGYSRSNCVRSYEYDY (SEQ ID NO: 26);
CDR1 comprising amino acid sequence GRTFSSRA (SEQ ID NO: 34); CDR2 comprising amino acid sequence SWTGSPY (SEQ ID NO: 35); and CDR3 comprising amino acid sequence AATSEHVMLVVTTRGGYDY (SEQ ID NO: 36); or
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13); and wherein the four VHH FRs are camelid anti-hIL-6 VHH FRs. In an embodiment of the polypeptide, the four camelid anti-hIL-6 VHH FRs comprise the following: FR1 comprises 25 consecutive amino acids comprising Q-X₁-Q-L-X₂-E-X₃-G-G-G-X₄-V-Q-X₅-G-X₆-S-L-X₇-L-S-C-A-A-S(SEQ ID NO: 37), wherein X₁ is L or V; X₂ is A or V; X₃ is T or S; X₄ is L or S; X₅ is P or A; X₆ is G or D; X₇ is R, T or G; FR2 comprises 18 consecutive amino acids comprising X₁-G-W-F-R-Q-A-P-G-K-E-R-E-X₂-X₃-X₄-X₅-X₆ (SEQ ID NO: 38), wherein X₁ is V, M, or I; X₂ is F or G; X₃ is I or V; X₄ is S or A; X₅ is C, A or V; X₆ is I or L; FR3 comprises 37-39 consecutive amino acids comprising Y-X₁-D-S-V-K-G-R-F-T-I-S-X₂-D-X₃-X₄-K-X₅-T-X₆-X₇-L-Q-M-N-S-L-K-P-E-D-T-X₈-X₉-Y-Y-C-A-A (SEQ ID NO: 39), wherein X₁ is A, T, or V; X₂ is R or G; X₃ is Y, N, or D; X₄ is A or D; X₅ is S, N, or D; X₆ is V or A; X₇ is S, F, or Y; X₈ is G or A; X₉ is V or T; and FR4 comprises 11 consecutive amino acids comprising W-X₁-Q-G-T-Q-V-T-V-S-S(SEQ ID NO: 40), wherein X₁ is D or G.

In another aspect of the present invention, a polypeptide that specifically binds to and neutralizes human interleukin-6 (hIL-6) cytokine, wherein the polypeptide or a hIL-6-binding portion thereof has at least 90% sequence identity to a sequence selected from the group consisting of SEQ ID NOs: 1, 3, 5, 7 and 9 is provided.

In another aspect of the present invention, a polypeptide that specifically binds to and neutralizes human interleukin-6 (hIL-6) cytokine, wherein the polypeptide or a hIL-6-binding portion thereof has at least 95% sequence identity to a sequence selected from the group consisting of SEQ ID NOs: 1, 3, 5, 7 and 9 is provided.

In embodiments of the above polypeptides, conservative amino acid substitutions in the polypeptide comprise the at least 90% or the at least 95% amino acid sequence identity.

In another aspect of the present invention, a polypeptide that specifically binds to and neutralizes human interleukin-6 (hIL-6) cytokine, wherein the polypeptide or a hIL-6-binding portion thereof comprises a sequence selected from the group consisting of SEQ ID NOs: 1, 3, 5, 7 and 9 is provided.

In an embodiment of the above-delineated polypeptides and/or embodiments thereof, the polypeptide is a camelid-derived single domain anti-hTL-6 VHH antibody. In an embodiment, the polypeptide is in the form of a dimer or multimer. In an embodiment, the polypeptide is in the form of a homodimer. In embodiments, the dimer, homodimer, or multimer comprises the hIL-6 binding polypeptides separated by a spacer or linker. In an embodiment, the polypeptide includes one or more epitope tag sequences specifically bindable by an anti-epitope tag antibody or binding portion thereof. In an embodiment, the polypeptide is linked to an immunoglobulin Fc domain.

In an aspect of the present invention, an isolated polynucleotide encoding the polypeptide as delineated above and/or embodiments thereof is provided.

In an aspect of the present invention, a pharmaceutical composition is provided comprising an effective amount of the above delineated polypeptide or a hIL-6-binding fragment thereof, or the above delineated isolated polynucleotide, and a pharmaceutically acceptable excipient, carrier, or diluent.

In an embodiment of any of the above-delineated aspects and/or embodiments thereof, the polypeptide neutralizes human interleukin-6 (hIL-6) activity.

In an aspect of the present invention, a method of inhibiting or abrogating interleukin-6 (IL-6)-induced STAT3 activation in a subject is provided, in which the method involves administering to a subject in need thereof an effective amount of the pharmaceutical composition of any of the above-delineated aspects and/or embodiments thereof, thereby inhibiting IL-6-induced STAT3 activation in the subject. In an embodiment of the method, IL-6-induced STAT3 activation is inhibited or abrogated in hepatocytes of the subject.

In an aspect of the present invention, a kit comprising the binding protein, the polypeptide, the dimeric or multimeric polypeptide, or the pharmaceutical composition of any of the above-delineated aspects and/or embodiments thereof, for treating or protecting against an interleukin-6 (IL-6)-mediated disease, disorder, condition, pathology, or infection and/or the symptoms thereof, and optionally comprising instructions for use.

In another aspect of the present invention, a method of detecting interleukin 6 (IL-6) or a peptide thereof in a sample is provided in which the method involves contacting the sample with at least one detectably labeled VHH binding protein or an antigen binding fragment thereof that specifically binds to IL-6 or a peptide thereof, wherein the binding protein or the antigen binding fragment thereof comprises three Complementarity Determining Regions (CDRs), CDR1, CDR2 and CDR3 structurally positioned between four framework (FR) regions (FR1-FR4) as follows: FR1-CDR1-FR2-CDR2-FR3-CDR3-FR4; wherein the three CDRs are selected from:
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GRPFSSFA (SEQ ID NO: 31); CDR2 comprising amino acid sequence TWSRGTTH (SEQ ID NO: 32); and CDR3 comprising amino acid sequence AAADGWKVVSTASPAYDY (SEQ ID NO: 33);
CDR1 comprising amino acid sequence GFTLAYYG (SEQ ID NO: 24); CDR2 comprising amino acid sequence SSSDLSTY (SEQ ID NO: 25); and CDR3 comprising amino acid sequence GTWDLKFGYSRSNCVRSYEYDY (SEQ ID NO: 26);
CDR1 comprising amino acid sequence GRTFSSRA (SEQ ID NO: 34); CDR2 comprising amino acid sequence SWTGSPY (SEQ ID NO: 35); and CDR3 comprising amino acid sequence AATSEHVMLVVTTRGGYDY (SEQ ID NO: 36); or
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13); and wherein FR1 comprises 25 consecutive amino acids comprising Q-X₁-Q-L-X₂-E-X₃-G-G-G-X₄-V-Q-X₅-G-X₆-S-L-X₇-L-S-C-A-A-S(SEQ ID NO: 37), wherein X₁ is L or V; X₂ is A or V; X₃ is T or S; X₄ is L or S; X₅ is P or A; X₆ is G or D; X₇ is R, T or G; FR2 comprises 18 consecutive amino acids comprising X₁-G-W-F-R-Q-A-P-G-K-E-R-E-X₂-X₃-X₄-X₅-X₆ (SEQ ID NO: 38), wherein X₁ is V, M, or I; X₂ is F or G; X₃ is I or V; X₄ is S or A; X₅ is C, A or V; X₆ is I or L; FR3 comprises 37-39 consecutive amino acids comprising Y-X₁-D-S-V-K-G-R-F-T-I-S-X₂-D-X₃-X₄-K-X₅-T-X₆-X₇-L-Q-M-N-S-L-K-P-E-D-T-X₈-X₉-Y-Y-C-A-A (SEQ ID NO: 39), wherein X₁ is A, T, or V; X₂ is R or G; X₃ is Y, N, or D; X₄ is A or D; X₅ is S, N, or D; X₆ is V or A; X₇ is S, F, or Y; X₈ is G or A; X₉ is V or T; and FR4 comprises 11 consecutive amino acids comprising W-X₁-Q-G-T-Q-V-T-V-S-S(SEQ ID NO: 40), wherein X₁ is D or G; under conditions for the binding protein to interact with IL-6; and measuring the level of binding of the binding protein to IL-6 in the sample relative to a control to detect or identify the presence of IL-6 in the sample. In an embodiment of the method, the sample is selected from blood, peripheral blood, serum, plasma, cerebrospinal fluid, urine, saliva, sputum, tears, stool, or synovial fluid.

In an embodiment of the above-delineated aspects and/or embodiments thereof related to the isolated polynucleotide, pharmaceutical compositions comprising the isolated polynucleotide, and methods involving the use of the isolated polypeptide and/or a pharmaceutical composition comprising the isolated polypeptide, the polynucleotide comprises mRNA. In an embodiment, the mRNA is formulated with a delivery agent. In an embodiment, the delivery agent comprises one or more of nanoparticles, lipid nanoparticles, ionizable lipids; biodegradable ionizable lipids; polymeric materials, polyethyleneimines (PEIs), poly(glycoamidoamine) polymers, poly(glycoamidoamine) polymers modified with fatty chains, poly(O-amino)esters (PBAEs), polymethacrylates; dendrimers, polyamidoamine (PAMAM), polypropylenimine-based dendrimers, PAMAM (generation 0) dendrimer co-formulated with poly(lactic-co-glycolic acid) (PLGA) and ceramide-PEG; cell penetrating peptides; cationic lipids; or zwitterionic lipids.

In an embodiment of any of the above-delineated methods and embodiments thereof, the polypeptide, any composition or pharmaceutical composition thereof, is administered to the subject prior or subsequent to an IL-6-mediated or induced disease, pathology, disorder, condition, infection, and the like.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). The following terms have the meanings ascribed to them below, unless specified otherwise.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in the specification and claim(s) herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions and products of the present disclosure can be used to achieve methods of the present disclosure.

Unless specifically stated or obvious from context, as used herein, the term “about” or “approximately” means within an acceptable error range for the particular value as determined by one of ordinary skill in the art, which will depend, in part, on how the value is measured or determined, i.e., the limitations of the measurement system. For example, “about” can mean within 1 standard deviation or more than 1 standard deviation, e.g., 2 standard deviations of the mean, as typically practiced in the art. Alternatively, and without intending to be limiting, “about” can mean a range of up to 20%, up to 10%, up to 5%, up to 2%, or up to 1% of a given value. Alternatively, and particularly for biological systems or processes, the term can mean within an order of magnitude, e.g., within 5-fold, within 3-fold, within 2.5-fold, or within 2-fold of a value. Where particular values are described in the application and claims, unless otherwise stated, the term “about” means within an acceptable error range for the particular value.

Reference herein to “some embodiments,” “an embodiment,” “one embodiment” or “other embodiments” means that a particular feature, structure, or characteristic described in connection with the embodiments is included in at least some embodiments, but not necessarily all embodiments, of the disclosure.

By “agent” is meant any small molecule chemical compound, antibody, nucleic acid molecule, or polypeptide (e.g., antibody or VHH antibody), or fragments thereof.

By “ameliorate” is meant decrease, reduce, diminish, suppress, attenuate, arrest, or stabilize the development or progression of a disease or pathology.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antibody” is meant any immunoglobulin polypeptide, or fragment thereof, having immunogen or antigen binding ability. Antibody structure is well known in the art. Briefly, the variable (V) regions or domains of antibody heavy (H) and light (L) chains contain Complementarity-Determining Regions (CDRs), which bind to specific antigens or immunogens (e.g., protein antigens or immunogens). CDRs are situated within framework (FR) sequences of the V regions of the heavy (V_{H}) and light chains (V_{L}) of an antibody. CDRs are the most variable parts of antibodies and are critical components in the diversity of antigen specificities of antibodies produced by B lymphocytes. In general, three CDRs (CDR1, CDR2 and CDR3) are arranged consecutively in a V domain of an antibody. Because a VHH, such as a camelid VHH, is essentially a single chain antibody polypeptide, it contains three CDRs that bind to an antigen or target protein such as human interleukin-6 (hIL-6) in the context of four framework (FR) regions, as follows: FR1-CDR1-FR2-CDR2-FR3-CDR3-FR4. Because most of the sequence variability associated with immunoglobulins and antigen binding is found in the CDRs, these regions are sometimes referred to as hypervariable regions. Typically, CDR1, CDR2 and CDR3 of VHHs contribute to and/or do not interfere with antigen binding. The CDRs of a number of anti-hIL-6 VHHs described herein are shown, for example, in Tables 1 and 3 and FIG. 2 herein.

A “camelid VHH framework region (FR)” refers to the structural FR portions or components of a camelid VHH antibody or binding molecule, namely, FR1, FR2, FR4 and FR4, that positionally and structurally support the three CDR components, namely, CDR1, CDR2 and CDR3 of a VHH polypeptide, as described above. Similar to the FRs in conventional antibody polypeptides, the respective FR regions (FR1, FR2, FR3 and FR4) of the anti-hIL6 VHH polypeptides described herein are highly similar in sequence not only among different IL6-(hIL6) binding VHHs but also among camelid VHH polypeptides that bind to other antigens, e.g., unrelated VHH polypeptides. (See, e.g., L. S. Mitchell and L. J. Colwell, 2018, Proteins, 86(7): 697-706 and A. M. Vattekatte et al., March, 2020, PeerJ., 6(8):e8408. DOI: 10.7717/peerj.8408). Accordingly, the FR regions FR1, FR2, FR3 and FR4 of different VHHs do not vary significantly in sequence. By way of example, the below FR sequences (SEQ ID NOS 47-50, respectively, in order of appearance) of the VHH in the above-mentioned publication of Mitchell and Colwell are highly similar to the FR sequences of other VHHs, including the anti-hIL6 VHH polypeptides described herein.

FR1

| | Position # | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| AA | Q | V | Q | L | Q/V | E | S | G | G | G | L/S | V | Q |

| | Position # | | | | | | | | | | | |
|---|---|---|---|---|---|---|---|---|---|---|---|---|
| | 14 | 15 | 16 | 17 | 18 | 19 | 20 | 21 | 22 | 23 | 24 | 25 |
| AA | A/P | G | G | S | L | R | L | S | C | A | A | S |

FR2

| | Position # | | | | | | | | | | | | | |
|---|---|---|---|---|---|---|---|---|---|---|---|---|---|---|
| | 36 | 37 | 38 | 39 | 40 | 41 | 42 | 43 | 44 | 45 | 46 | 47 | 48 | 49 |
| AA | W | F/Y/V | R | Q | A | P | G | K | E/C/G | R/L | E | F/G/L/W | V | A/S/T |

FR3

| | Position # | | | | | | | | | | | | | | | | | | |
|---|---|---|---|---|---|---|---|---|---|---|---|---|---|---|---|---|---|---|---|
| | 60 | 61 | 62 | 63 | 64 | 65 | 66 | 67 | 68 | 69 | 70 | 71 | 72 | 73 | 74 | 75 | 76 | 77 | 78 |
| AA | Y | A/Q/ | D/E | S | V/A | K | G | R | F | T/A | I/V | S | R/Q | D | N/K | A | K/A | N | T |
| | | T/V | | | | | | | | | | | | | | | | | |

| | Position # | | | | | | | | | | | | | | | | | |
|---|---|---|---|---|---|---|---|---|---|---|---|---|---|---|---|---|---|---|
| | 79 | 80 | 81 | 82 | 83 | 84 | 85 | 86 | 87 | 88 | 89 | 90 | 91 | 92 | 93 | 94 | 95 | 96 |
| AA | V/L/M | Y | L | Q | M | N/D | S/N | L | K/R | P | E/D | D | T | A/G | V/I/T/M | Y | Y | C |

FR4

| | Position # | | | | | | | | | | |
|---|---|---|---|---|---|---|---|---|---|---|---|
| | 117 | 118 | 119 | 120 | 121 | 122 | 123 | 124 | 125 | 126 | 127 |
| AA | W | G | Q | G | T | Q | V | T | V | S | S |

It will be appreciated that the amino acid position numbers of the VHH FRs shown above are approximate and may vary to some degree depending on VHH length and on the start and termination amino acid positions of the VHH CDRs. Thus, substantial similarities exist among the structural FRs of camelid VHHs, independent of antigen binding specificity.

A “chimeric antibody” refers to an antibody in which the constant region of an antibody of one species (e.g., rodent, mouse or rat) is replaced with that from a human to achieve a more human-like antibody. Chimeric antibodies may be recombinantly generated by combining the variable light and heavy chain regions obtained from antibody producing cells of one species with the constant light and heavy chain regions from another. In general, chimeric antibodies utilize rodent (or other species, such as rabbit or camelid) variable regions and human constant regions in order to produce an antibody with predominantly human constant domains. The production of chimeric antibodies is well known in the art, and may be achieved by standard means, for example, as described in U.S. Pat. No. 5,624,659, incorporated fully herein by reference.

By “binding to” a molecule is meant having a physicochemical affinity for a molecule (e.g., a protein or protein antigen) or a region of the molecule, e.g., an epitope or antigenic determinant. Binding may be measured by any of the methods practiced in the art, e.g., using an antibody binding assay (e.g., ELISA) or an in vitro translation binding assay.

“Detect” refers to identifying or determining the presence, absence or amount of an analyte, compound, or agent to be detected.

By “detectable label” is meant a compound, substance, or composition that, when linked to a molecule of interest, renders the latter detectable, via spectroscopic, photochemical, biochemical, immunochemical, or chemical means. For example, useful labels include, without limitation, radioactive isotopes, magnetic beads, metallic beads, colloidal particles, luminescent agents, fluorescence agents, chemiluminescent agents, colorimetric agents, electron-dense reagents, enzyme-substrate agents, (for example, as commonly used in an ELISA), biotin, digoxigenin, or haptens.

By “disease” is meant any condition, disorder, or pathology that damages or interferes with the normal function of a cell, tissue, or organ. Examples of diseases, disorders, pathologies, or infections associated with or caused by excess amounts, levels, or production of IL-6, or with dysregulation of IL-6 and/or IL-6 signaling, include, without limitation, infections (e.g., viral or bacterial infections); oncological diseases (cancers, carcinomas, tumors, and the like), e.g., cholangiocarcinoma, ovarian cancer, and multiple myeloma; immune-mediated diseases (autoimmune diseases and inflammatory diseases), e.g., adult rheumatoid arthritis, juvenile idiopathic arthritis, Castleman's disease, secondary amyloidosis, polymyalgia rheumatic, adult onset Still's disease, polymyositis, systemic sclerosis, large vessel vasculitis lupus erythematosus, Crohn's disease, irritable bowel disease (IBD), Sjogren's syndrome; steroid refractory Graft versus Host Disease in transplantation; type 2 diabetes, obesity and schizophrenia.

“Cytokine storm” (CS) and “cytokine release syndrome” (CRS) both refer to life-threatening systemic inflammatory syndromes involving elevated levels of circulating cytokines, such as IL-6, and immune-cell hyperactivation that can be triggered by various therapies, pathogens, cancers, autoimmune conditions, infections, diseases, and monogenic disorders (i.e., disorders caused by variation in a single gene, which are typically recognized by their familial inheritance patterns. Examples include sickle cell anemia, cystic fibrosis, Huntington disease, and Duchenne muscular dystrophy). (Fajgenbaum, D. C. and June, C. H., 2020, N Engl J Med., 383:2255-2273). CS is a physiological reaction in humans and other animals in which the innate immune system causes an uncontrolled and excessive release of pro-inflammatory signaling molecules (cytokines), e.g., IL-6. Cytokines are normally part of the body's immune response to infection, but their sudden release in large quantities can cause multisystem organ failure and death. Cytokine storms can be caused by a number of infectious and non-infectious etiologies, especially viral respiratory infections such as H5N1 influenza, SARS-CoV-1, and SARS-CoV-2 (Covid-19) virus agents, as well as Adult Respiratory Distress Syndrome (ARDS). The non-infectious condition graft-versus-host disease may be another cause of CS. In CS, viruses can invade lung epithelial cells and alveolar macrophages in which viral nucleic acid is produced. This stimulates the infected cells to release cytokines and chemokines, activating macrophages, dendritic cells, and other cell types.

By “effective amount” is meant the amount of a required to ameliorate, or optimally eliminate, the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

An “epitope tag” refers to a peptide or amino acid sequence (e.g., an epitope) that is fused, linked, or coupled to a protein, such as a recombinant protein produced by recombinant techniques, and that can be specifically bound by an antibody, e.g., an anti-tag monoclonal antibody or binding molecule that is directed to or generated against the tag peptide or amino acid sequence. Epitope tags are typically short peptide sequences (e.g., from about 5-30 amino acids, or sometimes up to 40 amino acids, that are selected because high-affinity antibodies can be reliably produced in many different species. Such anti-epitope tag antibodies are optimally not cross-reactive with other human peptides or polypeptides and typically do not generate an antibody response, e.g., an anti-tag antibody response, when administered or delivered to a subject. An epitope tag sequence that is fused to a protein provides for the detection and/or purification of the protein using an antibody, e.g., a monoclonal antibody, that specifically binds to the epitope tag. In an embodiment, the protein to which an epitope tag is fused, linked, or coupled is an antibody or VIII protein, e.g., a recombinantly produced antibody or VHH protein. In an embodiment, the VHH is an anti-hIL-6 VHH antibody. In an embodiment, the protein, or a dimeric or multimeric form thereof, may include one or more epitope tags. In an embodiment, an epitope tag is coupled to the amino (NH) terminus of the protein, e.g., a VHH antibody as described herein. In an embodiment, an epitope tag is coupled to the carboxy (COOH) terminus of the protein, e.g., a VHH antibody as described herein. In an embodiment, an epitope tag is coupled to the NH and the COOH termini of the protein, e.g., a VHH antibody as described herein. In an embodiment, a dimeric or multimeric form of the protein includes one or more, e.g., two, three or four, epitope tags linked to one or more of the VHHs comprising the dimeric or multimeric form of the protein. Such epitope tags may be coupled to the VHH components at locations within the dimer or multimer molecule, or at the NH and/or COOH termini of the molecule. In some embodiments, two or more epitope tags may be coupled to a VHH protein in tandem within or at the termini of the VHH protein or dimeric or multimeric form thereof. An epitope tag sequence such as those described herein may be bound by anti-epitope tag antibodies, forming complexes which may facilitate clearance of the protein containing the tags from the body or system. (See, also, B. Brizzard and R. Chubet, 2001, Curr Protoc Neurosci., Chapter 5, Unit 5.8; DOI: 10.1002/0471142301.ns0508s00; R. Hernan et al., 2000, Biotechniques, 28(4):789-793; C. E. Fritze et al., 2000, Meths Enzymol., 327:3-16; doi: 10.1016/s0076-6879(00)27263-7; A. Einhauer et al., 2001, J Biochem Biophys Methods, 49(1-3):455-65, doi: 10.1016/s0165-022x(01)00213-5)).

Other molecules may serve as protein, amino acid sequence, or polynucleotide tags that are fused, linked, or coupled to a protein, such as a recombinant protein produced by recombinant techniques, e.g., a hIL-6 VHH antibody described herein. In an embodiment, the tag can be specifically bound by an antibody, e.g., an anti-tag monoclonal antibody or binding molecule that is directed to or generated against the tag peptide or amino acid sequence. Examples of tags include, without limitation, FLAG tags (peptide sequence DYKDDDDK (SEQ ID NO: 51) recognized by an anti-FLAG antibody), polyHistidine (His) tags (5-10 histidine residues (SEQ ID NO: 52) (HHHHHH (SEQ ID NO: 53)) bound by a nickel or cobalt chelate), E-tag, a peptide comprising amino acid sequence GAPVPYPDPLEPR (SEQ ID NO: 42) recognized by an antibody; an immunoglobulin Fc region or portion thereof, e.g., having effector or modulator function (Fc tag). In particular, Fc tags comprise a domain (effector domain) of an immunoglobulin molecule, e.g., IgG, which can be genetically linked to a peptide or protein. Fc fusion proteins (also known as Fc chimeric fusion proteins, Fc-Igs, Ig-based chimeric fusion proteins, and Fc-tag proteins) are composed of an Ig Fc domain that is fused, linked, or coupled (e.g., by recombinant techniques) to a peptide or protein, such as an anti-hIL-6 VHH antibody described herein. The Fc domain portion of the fusion protein confers an advantageous characteristic to the protein, particularly in vivo, by greatly prolonging the half-life of the protein in plasma following administration to a subject. In an embodiment, an anti-hIL-6 VHH antibody fused to an Fc region or Fc tag provides improved therapeutic efficacy as a biotherapeutic agent or drug. Fc fusion proteins also have uses in in vitro methods, including, e.g., immunohistochemistry (IHC), flow cytometry (FC), protein binding assays and use as microarray baits. In these applications, the Fc domain serves as a support to which proteins can be attached while retaining their native biological activity. In addition, the Fc domain can improve the in vivo and in vitro solubility and stability of the protein or peptide molecule to which it is coupled, fused, linked, or attached.

A “framework (FR) region” or “FR region” includes amino acid residues that are adjacent to the CDRs in V_{H}, and V_{L} regions, and in VHHs. For example, FR region residues may be present in VHHs as described herein, camelid antibodies (VHHs), human antibodies, rodent-derived antibodies (e.g., murine and rat antibodies), humanized antibodies, primatized antibodies, chimeric antibodies, antibody fragments (e.g., Fab fragments), VHHs, single-chain antibody fragments (e.g., scFv fragments), antibody domains, and bispecific antibodies, among others.

By “fragment” is meant a portion of a polypeptide or nucleic acid molecule. This portion contains, preferably, at least 10%, 20%, 30%, 40%, 50%, 60%, 70%, 80%, 90%, or 95% of the entire length of the reference nucleic acid molecule or polypeptide, including percent values between those enumerated. A fragment may contain 10, 20, 30, 40, 50, 60, 70, 80, 90, or 100, 200, 300, 400, 500, 600, 700, 800, 900, or 1000 nucleotides or amino acids. In an embodiment, a fragment or portion possesses or retains activity or function of the polypeptide from which it is derived.

The term “homodimer” refers to a VHH binding molecule, antibody, or nanobody (“VHH”) which comprises two of the same VHH components that are separated (i.e., joined together) by a spacer or linker, such as a flexible spacer or linker peptide sequence. In an embodiment, the VHH homodimer comprises two JYK-D12 anti-hIL6 VHH antibodies (also termed “VCR-108” herein).

The term “humanized” antibodies refers to forms of non-human (e.g., murine) antibodies, camelid-derived single domain antibody (sdAb) binding molecules, which are comprised of the heavy chain variable (V_{H}) region of heavy-chain-only antibodies (Abs) or VHHs. Humanized antibodies include chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, a humanized antibody or VHH may comprise substantially all of at least one variable domain (or two variable domains in the case of non-VHH antibodies), in which all or substantially all of the CDR regions correspond to those of a non-human immunoglobulin. All or substantially all of the FR regions of a humanized antibody may also be derived from a human immunoglobulin sequence. In the case of non-VHH antibodies, a VHH or a humanized antibody can also comprise at least a portion of an immunoglobulin constant region (Fc), which may be that of a human immunoglobulin consensus sequence. Techniques and protocols for humanizing antibodies (as well as VHHs) are known and practiced in the art, as described, for examples, in Riechmann et al., Nature, 332:323-7, 1988; Kasmiri et al., Methods, 36(1):25-34, 2005; U.S. Pat. Nos. 5,530,101; 5,585,089; 5,693,761; 5,693,762; and U.S. Pat. No. 6,180,370 to Queen et al; EP239400; WO 1991/09967; U.S. Pat. No. 5,225,539; EP592106; and EP519596, the contents of which are incorporated herein by reference. Humanized antibodies or VHHs are molecularly engineered to contain even more human-like immunoglobulin domains, and incorporate only the CDRs of the VHH or animal-derived monoclonal antibody by carefully examining the sequence of the hyper-variable loops of the V regions of the monoclonal antibody or VHH, and fitting them to the structure of the human antibody chains. This process is routinely and commonly carried out by one having skill in the art. See, e.g., U.S. Pat. No. 6,187,287, the contents of which are incorporated by reference herein.

An “interleukin 6 (IL-6)” polypeptide or protein refers to a polypeptide or protein sequence having at least 85%, at least 90%, at least 95%, or at least 99% amino acid sequence identity to the human IL-6 (hIL-6) amino acid sequence (212 amino acids) as set forth below (NCBI Reference Sequence: NP_000591.1):

(SEQ ID NO: 54)
MNSFSTSAFG PVAFSLGLLL VLPAAFPAPV PPGEDSKDVA
APHRQPLTSS ERIDKQIRYI LDGISALRKE TCNKSNMCES
SKEALAENNL NLPKMAEKDG CFQSGFNEET CLVKIITGLL
EFEVYLEYLQ NRFESSEEQA RAVQMSTKVL IQFLQKKAKN
LDAITTPDPT TNASLLTKLQ AQNQWLQDMT THLILRSFKE
FLQSSLRALR QM

Portions or fragments of the IL-6 polypeptide sequence, in particular, those that specifically bind to the anti-IL-6 VHHs described herein are encompassed.

An “interleukin 6 (IL-6)” polynucleotide refers to a polynucleotide or nucleic acid sequence having at least 85%, at least 90%, at least 95%, or at least 99% nucleic acid sequence identity to the human IL-6 (hIL-6) polynucleotide sequence as set forth below (NCBI Reference Sequence: NM_000600.5):

| (SEQ ID NO: 55) | |
|---|---|
| 1 | attctgccct cgagcccacc gggaacgaaa gagaagctct atctcccctc caggagccca |
| 61 | gctatgaact ccttctccac aagcgccttc ggtccagttg ccttctccct ggggctgctc |
| 121 | ctggtgttgc ctgctgcctt ccctgcccca gtacccccag gagaagattc caaagatgta |
| 181 | gccgccccac acagacagcc actcacctct tcagaacgaa ttgacaaaca aattcggtac |
| 241 | atcctcgacg gcatctcagc cctgagaaag gagacatgta acaagagtaa catgtgtgaa |
| 301 | agcagcaaag aggcactggc agaaaacaac ctgaaccttc caaagatggc tgaaaaagat |
| 361 | ggatgcttcc aatctggatt caatgaggag acttgcctgg tgaaaatcat cactggtctt |
| 421 | ttggagtttg aggtatacct agagtacctc cagaacagat ttgagagtag tgaggaacaa |
| 481 | gccagagctg tgcagatgag tacaaaagtc ctgatccagt tcctgcagaa aaaggcaaag |
| 541 | aatctagatg caataaccac ccctgaccca accacaaatg ccagcctgct gacgaagctg |
| 601 | caggcacaga accagtggct gcaggacatg acaactcatc tcattctgcg cagctttaag |
| 661 | gagttcctgc agtccagcct gagggctctt cggcaaatgt agcatgggca cctcagattg |
| 721 | ttgttgttaa tgggcattcc ttcttctggt cagaaacctg tccactgggc acagaactta |
| 781 | tgttgttctc tatggagaac taaaagtatg agcgttagga cactatttta attattttta |
| 841 | atttattaat atttaaatat gtgaagctga gttaatttat gtaagtcata tttatatttt |
| 901 | taagaagtac cacttgaaac attttatgta ttagttttga aataataatg gaaagtggct |
| 961 | atgcagtttg aatatccttt gtttcagagc cagatcattt cttggaaagt gtaggcttac |
| 1021 | ctcaaataaa tggctaactt atacatattt ttaaagaaat atttatattg tatttatata |
| 1081 | atgtataaat ggtttttata ccaataaatg gcattttaaa aaattca |

An “interleukin 6 (IL-6)” polypeptide or protein refers to a polypeptide or protein sequence having at least 85%, at least 90%, at least 95%, or at least 99% amino acid sequence identity to the mouse IL-6 (mIL-6) amino acid sequence (211 amino acids) as set forth below (NCBI Reference Sequence: NP_112445):

| (SEQ ID NO: 56) | |
|---|---|
| 1 | MKFLSARDFH PVAFLGLMLV TTTAFPTSQV RRGDFTEDTT PNRPVYTTSQ VGGLITHVLW |
| 61 | EIVEMRKELC NGNSDCMNND DALAENNLKL PEIQRNDGCY QTGYNQEICL LKISSGLLEY |
| 121 | HSYLEYMKNN LKDNKKDKAR VLQRDTETLI HIFNQEVKDL HKIVLPTPIS NALLTDKLES |
| 181 | QKEWLRTKTI QFILKSLEEF LKVTLRSTRQ T |

An “interleukin 6 (IL-6)” polynucleotide refers to a polynucleotide or nucleic acid sequence having at least 85%, at least 90%, at least 95%, or at least 99% nucleic acid sequence identity to the mouse IL-6 (mIL-6) polynucleotide sequence as set forth below (NCBI Reference Sequence: NM_031168.2):

| (SEQ ID NO: 57) | |
|---|---|
| 1 | aaatatgaga ctggggatgt ctgtagctca ttctgctctg gagcccacca agaacgatag |
| 61 | tcaattccag aaaccgctat gaagttcctc tctgcaagag acttccatcc agttgccttc |
| 121 | ttgggactga tgctggtgac aaccacggcc ttccctactt cacaagtccg gagaggagac |
| 181 | ttcacagagg ataccactcc caacagacct gtctatacca cttcacaagt cggaggctta |
| 241 | attacacatg ttctctggga aatcgtggaa atgagaaaag agttgtgcaa tggcaattct |
| 301 | gattgtatga acaacgatga tgcacttgca gaaaacaatc tgaaacttcc agagatacaa |
| 361 | agaaatgatg gatgctacca aactggatat aatcaggaaa tttgcctatt gaaaatttcc |
| 421 | tctggtcttc tggagtacca tagctacctg gagtacatga agaacaactt aaaagataac |
| 481 | aagaaagaca aagccagagt ccttcagaga gatacagaaa ctctaattca tatcttcaac |
| 541 | caagaggtaa aagatttaca taaaatagtc cttcctaccc caatttccaa tgctctccta |
| 601 | acagataagc tggagtcaca gaaggagtgg ctaaggacca agaccatcca attcatcttg |
| 661 | aaatcacttg aagaatttct aaaagtcact ttgagatcta ctcggcaaac ctagtgcgtt |
| 721 | atgcctaagc atatcagttt gtggacattc ctcactgtgg tcagaaaata tatcctgttg |
| 781 | tcaggtatct gacttatgtt gttctctacg aagaactgac aatatgaatg ttgggacact |
| 841 | attttaatta tttttaattt attgataatt taaataagta aactttaagt taatttatga |
| 901 | ttgatattta ttatttttat gaagtgtcac ttgaaatgtt atatgttata gttttgaaat |
| 961 | gataacctaa aaatctattt gatataaata ttctgttacc tagccagatg gtttcttgga |
| 1021 | atgtataagt ttacctcaat gaattgctaa tttaaatatg tttttaaaga aatctttgtg |
| 1081 | atgtattttt ataatgttta gactgtcttc aaacaaataa attatattat atttaaaaac |
| 1141 | c |

Interleukin 6 (IL-6), in particular, human IL-6 (hIL-6), is a cytokine that functions in inflammation and the maturation of B cells. In addition, the IL-6 protein has been shown to be an endogenous pyrogen capable of inducing fever in people with autoimmune diseases or infections. IL-6 is primarily produced at sites of acute and chronic inflammation, where it is secreted into the serum and induces a transcriptional inflammatory response through interleukin 6 receptor-alpha (IL-6Rα). The functioning of the gene coding for IL-6 is implicated in a wide variety of inflammation-associated disease states, including susceptibility to diabetes mellitus and systemic juvenile rheumatoid arthritis. Elevated levels of the encoded protein have been found in patients having virus infections, including the Covid-19 virus. Interleukin-6 is released by monocytes and macrophages in response to other inflammatory cytokines, which include interleukin-11 (IL-11), and tumor necrosis factor-beta (TNF-β). The IL-6 receptor is present on normal T-lymphocytes in the resting phase, normal activated B-cells, and cells in the myeloid and hepatic cell lines. It is also found on B cells modified by the Epstein-Barr virus.

Interleukin-6 produces inflammatory effects by inducing the transcription of factors in multiple pathways of inflammation. These may originate with protein kinase C (PKC), cAMP/protein kinase A. and calcium release. IL-6 is a molecule with multiple forms and functions, depending on where it is secreted IL-6 is involved in the differentiation of T cells early in their development. It is required for progenitor cell development, and also for I-cell and NK cell activation. IL-6 is involved in aiding T-cell and NK to achieve pathogen lysis inside the cells.

Interleukin-6 promotes B cell differentiation and proliferation, as well as the formation of plasma cells from B cells. In addition, as a growth factor for these cells. IL-6 enhances IgA and IgG antibody release. The IL-6 cytokine is also vital for the development of red and white blood cells and platelets. The presence of IL-6 can lead to the activation of osteoclasts and osteoporosis and to the induction of the secretion of vascular endothelial growth factor (VEGF), which causes increased growth of blood vessels and vascular permeability in inflammation.

While IL-6 participates in the short-term defense against infection or injury and provides surveillance in the immune system against the source of inflammation, defective regulation of IL-6 results in disease. IL-6 deficiency has profound effects on immune activation and IgA antibody production. Moreover, IL-6 overexpression has equally important effects. Acting through different pathways, IL-6 creates an immunological imbalance between Th-17 cells and Treg cells, resulting in autoimmune pathology Defective IL-6 regulation may also produce lymphoid malignancies. IL-6 also may play an important role in the development of Kaposi's sarcoma, and multiple myeloma. In another of its biological roles, IL-6 is also used as a biological response modifier, e.g., to enhance the response to chemotherapy by stimulating the immune response in cancer.

An “interleukin-6 (IL-6)-mediated” or “IL-6-induced” disease, disorder, pathology, condition, or infection refers to one that is associated with or caused by the presence of the IL-6 cytokine, and/or excess amounts, levels, or production of IL-6, or with dysregulation of IL-6, the IL-6 pathway, and/or IL-6 signaling in a cell in vitro and/or in vivo in a subject. In some embodiments, an “IL-6-mediated” or “IL-6-induced” disease, disorder, pathology, or condition, includes a virus infection, such as SARS-Covid19, an inflammatory disease or disorder, or cancer, or Adult Respiratory Distress Syndrome (ARDS).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the terms “polynucleotide,” “DNA molecule” or “nucleic acid molecule” include both sense and anti-sense strands, cDNA, genomic DNA, recombinant DNA, RNA, mRNA, and wholly or partially synthesized nucleic acid molecules. A nucleotide “variant” is a sequence that differs from the recited nucleotide sequence in having one or more nucleotide deletions, substitutions or additions. Such modifications are readily introduced using standard mutagenesis techniques, such as oligonucleotide-directed site-specific mutagenesis as described, for example, in Adelman et al., 1983, DNA 2:183. Nucleotide variants are naturally-occurring allelic variants, or non-naturally occurring variants. Variant nucleotide sequences in various embodiments exhibit at least about 70%, 75%, 80%, 85%, 90%, 95%, 98%, 99%, or 100% sequence homology or sequence identity to the recited sequence. Such variant nucleotide sequences hybridize to the recited nucleotide sequence under stringent hybridization conditions. In one embodiment, “stringent conditions” refers to prewashing in a solution of 6×SSC, 0.2% SDS; hybridizing at 65° Celsius, 6×SSC, 0.2% SDS overnight; followed by two washes of 30 minutes each in 1×SSC, 0.1% SDS at 65° C., and two washes of 30 minutes each in 0.2×SSC, 0.1% SDS at 65° C.

By “isolated polynucleotide” is meant a nucleic acid (e.g., DNA, cDNA, RNA, mRNA) that is free of the genes, which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, e.g., mRNA, as well as a recombinant DNA that is part of a hybrid gene encoding additional polypeptide sequence.

The terms “protein”, “peptide” and “polypeptide” are used herein to describe any chain of amino acid residues, regardless of length or post-translational modification (for example, glycosylation or phosphorylation). Thus, these terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length proteins, which may be, but need not be, naturally occurring, as well as recombinantly or synthetically produced polypeptides that correspond to a full-length protein, or to particular domains or portions of a protein, which may be, but need not be, naturally occurring. The term also encompasses mature proteins which have an added amino-terminal methionine to facilitate expression in prokaryotic cells. The binding molecules of the invention are encoded by polynucleotides and can be chemically synthesized or synthesized by recombinant DNA methods.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

A “nanobody” as used herein also is used synonymously to refer to a single-domain antibody or VHH. A nanobody refers to an antibody fragment or portion which contains a single monomeric variable domain (V_{H}) naturally occurring in the Camelidae family or synthetically derived from the heavy chain of an antibody. Such single-domain binding molecules combine high antigen affinity in the absence of complement-dependent or cell-mediated cytotoxicity due to the lack of a constant (Fc) region in these molecules.

As used herein, “obtaining” as in “obtaining an agent” includes synthesizing, purchasing, deriving, producing, isolating, or otherwise acquiring the agent.

By “operably linked” is meant the connection between regulatory elements and one or more polynucleotides (genes) or a coding region. That is, gene expression is typically placed under the control of certain regulatory elements, including constitutive or inducible promoters, tissue-specific regulatory elements, and enhancers. A polynucleotide (gene or genes) or coding region is said to be “operably linked to” or “operatively linked to” or “operably associated with” the regulatory elements, meaning that the polynucleotide (gene or genes) or coding region is controlled or influenced by the regulatory elements. The one or more polynucleotides may be separated by spacers or linkers.

By “pathogen” is meant any harmful microorganism, bacterium, virus, fungus, or protozoan capable of interfering with the normal function of a cell. Pathogens may produce toxins, e.g., protein toxins, that intoxicate the cells, tissues and organs of a host or recipient organism and cause disease and pathology.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative or lowering alteration of at least 5%, 10%, 15%, 10%, 25%, 50%, 75%, or 100%.

By “reference” is meant a standard or control condition typically used as a comparator in an assay, test, experiment, or trial, as would be understood by one having skill in the pertinent art. In various nonlimiting embodiments, a reference or control is a different or nonpathogenic protein or cell, such as a normal cell, a cell having normal or non-aberrant IL-6 function or activity, a wild-type (unmutated or unaltered) protein, or a healthy (non-diseased) subject or individual.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound, molecule, antibody, or VHH that recognizes and binds a protein, peptide, or polypeptide (e.g., an amino acid sequence of the protein, peptide, or polypeptide), but which does not substantially recognize and bind other molecules in a sample, for example, a biological sample, which may contain the protein, peptide, or polypeptide that is specifically bound. In an embodiment, the VHHs as described herein specifically bind to the IL-6 protein. In an embodiment, the VHHs as described herein specifically bind to the IL-6 protein and neutralize activity associated with IL-6. In an embodiment, the IL-6 protein is human IL-6 (hIL-6).

“Nucleic acid” (also called polynucleotide herein) refers to deoxyribonucleotides or ribonucleotides and polymers thereof in either single- or double-stranded form. The term encompasses nucleic acids (polynucleotides) containing known nucleotide analogs or modified backbone residues or linkages, which are synthetic, naturally occurring, and non-naturally occurring, which have similar binding properties as a reference nucleic acid, and which are metabolized in a manner similar to the reference nucleic acid. Examples of such analogs include, without limitation, phosphorothioates, phosphoramidates, methyl phosphonates, chiral methyl phosphonates, 2-O-methyl ribonucleotides, and peptide-nucleic acids (PNAs). Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., 1991, Nucleic Acid Res, 19:081; Ohtsuka et al., 1985, J. Biol. Chem., 260:2600-2608; Rossolini et al., 1994, Mol. Cell Probes, 8:91-98). The term nucleic acid can be used interchangeably with gene, cDNA, mRNA, oligonucleotide, and polynucleotide.

Nucleic acid molecules or polynucleotides useful in the invention include any nucleic acid molecule or polynucleotide that encodes a polypeptide, e.g., a heteromultimeric binding molecule, of the invention or a component or portion thereof. Nucleic acid molecules useful in the methods of the invention include any polynucleotide or nucleic acid molecule that encodes a polypeptide e.g., heteromultimeric binding molecule, of the invention or a component or portion thereof that has substantial identity to the binding molecule. Such nucleic acid molecules need not be 100% identical with the nucleic acid sequence of the binding molecule, but will typically exhibit substantial identity. Polynucleotides having “substantial identity” to a binding molecule sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, 1987, Methods Enzymol. 152:399; Kimmel, A. R., 1987, Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “homologous” in their various grammatical forms in the context of polynucleotides means that a polynucleotide comprises a sequence that has a desired identity, for example, at least 60% identity, at least 70% sequence identity, at least 80%, at least 85% identity, at least 90% identity; and at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like.

Substantial identity of amino acid sequences, for example, the IL-6 binding polypeptides (anti-IL-6 VHH antibodies) refers to sequence identity between or among amino acid sequences of at least 60%, at least 65%, at least 70%, at least 75%, at least 80%, at least 85%, at least 88%, at least 90%, at least 95%, at least 98%, at least 99% or greater sequence identity. In embodiments, 100% identity between or among the amino acid sequences, e.g., the CDR1-3 sequences of the anti-hIL-6 VHHs as described herein is not required for binding of these polypeptides to IL-6 and/or neutralization of IL-6 activity. In a particular embodiment, variations between or among VHH amino acid sequences encompass one or more conservative amino acid substitutions in the sequence, for example, as shown in Tables 1-3 and FIG. 2 herein. In an embodiment, one or more conservative amino acid substitutions in an anti-hIL-6 VHH amino acid sequence may be in one or more CDR sequences, one or more FR sequences, or a combination thereof.

As will be appreciated by the skilled practitioner in the art, some amino acids in a VHH antibody can be modified without significantly altering antigen binding of the VHH antibody. For example, such amino acid sequence modification occurs frequently during in vivo affinity maturation of VHH antibodies, and the best mutations, e.g., for specific and/or high affinity binding to antigen, are positively selected for in the animal during the molecular production of antibodies. It is possible to isolate different VHH intermediates in the affinity maturation process that possess acceptable and specific antigen binding properties and that have significant variations in their CDR sequences.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

A “sample,” such as a biological sample for analysis or as used in the methods described herein, can be selected, without limitation, from blood, peripheral blood, serum, plasma, cerebrospinal fluid (CF), urine, saliva, sputum, tears, stool and synovial fluid. A sample may be a cell, tissue, or organ sample that may be prepared for analysis and use (e.g., dissociated, homogenized, and suspended in solution) by methods known in the art. A sample may be obtained or derived from a subject.

By “subject” is meant a mammal, including, but not limited to, a human or non-human mammal, such as, without limitation, a human, a non-human primate, or a bovine, equine, canine, ovine, or feline mammal. Other mammals include rabbits, goats, llamas, mice, rats, guinea pigs, camels and gerbils. In particular, a “subject” as used herein refers to a human subject, such as a human patient or individual. In some cases, the terms subject, patient and individual are used interchangeably herein. Subjects and patients may be male and/or female.

A “VHH binding molecule” or “VHH antibody,” or simply “VHH,” as referred to herein is, in general, a single domain immunoglobulin molecule (antibody) isolated from camelid animals (alpacas), e.g., as described in Maass, D. R., 2007, J. Immunol. Methods, 324(1-2):13-15). A VHH (or VHH antibody) corresponds to the heavy chain of a camelid antibody having a single variable domain (or single variable region), e.g., a camelid-derived single variable H (V_{H}) domain antibody. A VHH has a molecular weight (MW) of about 12-15 kDa. VHH technology is based on fully functional antibodies from camelids that lack light chains. These heavy-chain antibody molecules contain a single variable domain (VHH) and, typically, two constant domains (CH₂ and CH₃). See, e.g., Methods in Molecular Biology, “Single Domain Antibodies—Methods and Protocols,” Eds. D. Saerens and S. Muyldermans, Humana Press (Springer), 2012. A cloned (recombinantly produced) and isolated VHH domain is a stable polypeptide harboring the antigen-binding capacity of the original heavy-chain antibody. See, e.g., U.S. Pat. Nos. 5,840,526 and 6,015,695, each of which is incorporated by reference herein in its entirety.

VHHs are efficiently expressed in E. coli, coupled to detection markers, such as a fluorescent marker, or conjugated with enzymes. The small size of VHHs permits their binding to epitopes (antigenic determinants in antigen proteins), e.g., “hidden epitopes” that are not accessible to whole antibodies of much larger size. As a therapeutic, a VHH is capable of efficient penetration and rapid clearance. Its single domain nature allows a VHH to be expressed in a cell without a requirement for supramolecular assembly, as is needed for whole antibodies which are typically tetrameric (two heavy chains and two light chains, having a MW of about 150 kDa). VHHs are also exhibit stability over time and have a longer half-life versus non-VHH antibody molecules, which comprise disulfide bonds that are susceptible to chemical reduction or enzymatic cleavage. Similar to immunoglobulins, VHHs may be modified post-translationally, e.g. to add chemical linkers, detectable moieties, such as fluorescent dyes, enzymes, substrates, chemiluminescent moieties, etc., or specific binding moieties, such as streptavidin, avidin, or biotin, etc., for use in the compositions and methods described herein.

An anti-IL-6 VHH polypeptide that specifically binds to and/or neutralizes the activity of IL-6, may also be referred to as a “VHH-based neutralizing agent (VNA)” a “VNA polypeptide or protein” or a “VNA binding molecule,” or “nanobody.”

As used herein, the terms “treat,” treating,” “treatment,” and the like refer to reducing, diminishing, abating, alleviating, improving, ameliorating, or eliminating a disorder and/or symptoms associated therewith. It will be appreciated that, although not precluded, treating a disorder or condition does not require that the disorder, condition or symptoms associated therewith be completely eliminated. Diseases, disorders, pathologies, or infections associated with or caused by excess amounts, levels, or production of IL-6, or with dysregulation of IL-6 and/or IL-6 signaling and which are suitable for treatment with the anti-IL-6 VHHs described herein include, without limitation, infections (e.g., viral or bacterial infections); oncological diseases (cancers, carcinomas, tumors, and the like), e.g., cholangiocarcinoma, ovarian cancer, and multiple myeloma; immune-mediated diseases (autoimmune diseases and inflammatory diseases), e.g., adult rheumatoid arthritis, juvenile idiopathic arthritis, Castleman's disease, secondary amyloidosis, polymyalgia rheumatic, adult onset Still's disease, polymyositis, systemic sclerosis, large vessel vasculitis lupus erythematosus, Crohn's disease, irritable bowel disease (IBD), Sjogren's syndrome; steroid refractory Graft versus Host Disease in transplantation; type 2 diabetes, obesity and schizophrenia.

The term “multimeric binding molecule” refers in general to a multi-component protein or polypeptide containing two or more, same or different, VHH binding molecules, which are coupled or linked, e.g., via spacer (or linker) sequences, to each other and/or other components of the molecule. In an embodiment, the spacer or linker sequence is a flexible spacer or linker sequence. In an embodiment, without limitation, the spacer or linker sequence comprises (GGGGS)₃ (SEQ ID NO: 44). Multimeric binding molecules may be dimeric, in that the binding molecule contains two VHH polypeptides that bind to IL-6. The anti-IL-6 VHH polypeptides in a dimeric multimer may be the same or they may be different VHH polypeptides. In an embodiment, the anti-IL-6 VHH polypeptide is a dimer. In an embodiment, the anti-IL-6 VHH polypeptide is a homodimer in which the component VHH polypeptides are the same. In an embodiment, the homodimer comprises two JYK-D12 anti-IL-6 VHH molecules (also referred to as “nanobodies”), or closely related VHH polypeptides (e.g., as presented in Table 1). In an embodiment, the VHH polypeptide components of the dimer, homodimer, or multimer are separated by a spacer or linker, e.g., a flexible spacer or linker. The different anti-IL-6 VHH polypeptides in a multimeric binding molecule may bind to different regions, portions, or epitopes (e.g., non-overlapping epitopes) of IL-6. Alternatively, the multimeric binding molecules may be heteromultimeric, in that the binding molecule contains more than one, e.g., two, three, or four, different anti-IL-6 VHH polypeptides such as described herein. In some embodiments, a heteromultimeric binding molecule contains two or more different anti-IL-6 VHH polypeptides, each of which specifically binds to the IL-6 polypeptide, e.g., at different or non-overlapping epitopes. In embodiments, dimeric multimers and heteromultimeric binding molecules comprising two or more anti-IL-6 VHHs bind to and neutralize the activity of the IL-6 polypeptide.

As used herein, the terms “prevent,” “preventing,” “prevention,” “prophylactic treatment,” “protection” and the like refer to reducing the probability of developing a disorder or condition in a subject, who does not have, but who is at risk of, is susceptible to, or disposed to (e.g., genetically disposed to), developing a disease, disorder, pathology, or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of the first and last values.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Although various features of the present disclosure can be described in the context of a single embodiment, the features can also be provided in separate embodiments, or in any suitable combination or combination of embodiments. The section headings used herein are for organizational purposes only and are not intended to be limiting to the subject matter described.

The features of the present disclosure are set forth with particularity in the appended claims. The features and advantages of the present disclosure will be better understood and obtained by reference to the detailed description infra, which sets forth illustrative embodiments, in which the principles of the disclosure are utilized, and in view of the accompanying drawings as described hereinbelow.

### BRIEF DESCRIPTION OF THE DRAWINGS

FIG. 1 presents a graph related to binding activity of representative anti-hIL-6 VHH antibodies as described herein. The graph in FIG. 1 shows the results of binding analyses performed using an enzyme linked immunosorbent assay (ELISA) to assess the apparent binding affinities of representative, purified anti-IL-6 VHH antibodies to human IL-6 protein (hIL-6) coated on a solid substrate. The anti-IL-6 VHH antibodies analyzed in FIG. 1 represent members of one among four clonally-independent (i.e., derived from independent B cells) families of VHHs obtained from lymphocytes of camelids (alpacas) immunized with hIL-6. In FIG. 1, the labels (A) through (G) identify the specific anti-IL-6 VHH antibody in the legend with its corresponding IL-6 binding affinity plot on the graph.

FIG. 2 provides a sequence comparison table in which hIL-6-binding VHH polypeptide sequences of 125 or 126 amino acids in length are aligned. The CDR and FR regions of the VHH molecules are shown. In the sequences set forth in FIG. 2, linearly from left to right, framework 1 (FR1) is approximately 20 amino acids in length and encompasses amino acid residues 1 to 20; complementarity determining region 1 (CDR1) is approximately 9 amino acids in length and encompasses amino acid residues 21 to 29; framework 2 (FR2) is approximately 18 amino acids in length and encompasses amino acid residues 29-46; complementarity determining region 2 (CDR2) is approximately 9 amino acids in length and encompasses amino acid residues 47-54; framework 3 (FR3) is approximately 37 amino acids in length and encompasses amino acid residues 56-92; complementarity determining region 3 (CDR3) is approximately 23 amino acids in length and encompasses amino acid residues 93-115; and framework 4 (FR4) is approximately 11 amino acids in length and encompasses amino acid residues 116-126. It will be appreciated that the exact boundaries of the FRs and CDRs may be imprecise, as amino acid sequence variability is typically observed at and near the end of a FR and at and near the start of a hypervariable CDR. Anti-IL-6 VHH polypeptide sequences described herein (Example 1) and in Tables 3a and 3b, i.e., JYK-A1, JYK-A9, JYK-D12, and JYK-H9, are representative VHHs among the VHH sequences set forth in FIG. 2. The designation “sh” or “lh” in FIG. 2 indicates that the VHH, when produced as a homodimer, contains a short hinge (sh) or spacer (linker), or a long hinge (lh) or spacer (linker). FIG. 2 discloses SEQ TD NOS 93, 93, 94, 94, and 95-105, respectively, in order of appearance. FIG. 2 includes the following IL-6 binding VHHs containing complementarity determining regions (CDRs), CDR1, CDR2 and CDR3 the amino acid sequences of which are shown in Table 1:

TABLE 1

| VHH | | | | | | |
|---|---|---|---|---|---|---|
| (anti-IL- | | SEQ ID | | SEQ ID | | SEQ ID |
| 6 VHH) | CDR1 | NO: | CDR2 | NO: | CDR3 | NO: |
| XAX-C9 | GFTLDYYA | 11 | SSSDRSTY | 12 | GTWDLKWGYNISACVGSYEYDY | 13 |
| sh.ab1 | | | | | | |
| XAX-H12 | GFTLDYYA | 11 | SSSDRSTY | 12 | GTWDLKWGYNISACVGSYEYDY | 13 |
| sh.ab1 | | | | | | |
| XAX-H9 | GFTLDYYA | 11 | SSSDRSTY | 12 | GTWDLKWGYNISACVGSYEYDY | 13 |
| sh.ab1 | | | | | | |
| JYK-H9 | GFTLDYYA | 11 | SSSDRSTY | 12 | GTWDLKWGYNISACVGSYEYDY | 13 |
| sh.ab1 | | | | | | |
| JYK-A8 | GFTLDYYA | 11 | SSSDRSAY | 14 | GTWDLKWGYNISACVRSYEYDY | 15 |
| sh.ab1 | | | | | | |
| JYK-G1 | GFTLDYYA | 11 | SSSDLKTY | 16 | GTWDLKFGYNISACVGSYEYDY | 17 |
| sh.ab1 | | | | | | |
| XAX-E6 | GFTLDYYA | 11 | SSSDLKTY | 16 | GTWDLKFGYNISACVGSYEYDY | 17 |
| lh.ab1 | | | | | | |
| JYK-A1 | GFTLDYYA | 11 | SSSDLKTY | 16 | GTWDLKFGYNISACVGSYEYDY | 17 |
| lh.ab1 | | | | | | |
| JYK-F6 | GFTLDYYA | 11 | SSSDLKTY | 16 | GTWDLKFGYNISACVGSYEYDY | 17 |
| lh.ab1 | | | | | | |
| XAX-G8 | GFTLDYYA | 11 | SSSDLKTY | 16 | GTWDLKFGYNISACVGSYEYDY | 17 |
| lh.ab1 | | | | | | |
| JYK-G10 | GFALDYYA | 18 | SSSDLKTY | 16 | GTWDLKFGYNISACVGSYEYDY | 17 |
| lh.ab1 | | | | | | |
| XAX-B7 | GFTLDYYG | 19 | SSSDLKTY | 16 | GTWDLKFGYNITTCVRSSEYDY | 20 |
| lh.ab1 | | | | | | |
| XAX-H5 | GFTSDYYG | 21 | SSSDLKTY | 16 | GTWDLKFGYNITTCVRSSEYDY | 20 |
| lh.ab1 | | | | | | |
| XAX-C2 | GFTLDYYG | 19 | SSSDWSTY | 22 | GTWDLKFGYNRSNCVRSAEYDY | 23 |
| lh.ab1 | | | | | | |
| JYK-D12 | GFTLAYYG | 24 | SSSDLSTY | 25 | GTWDLKFGYSRSNCVRSYEYDY | 26 |
| sh.ab1 | | | | | | |

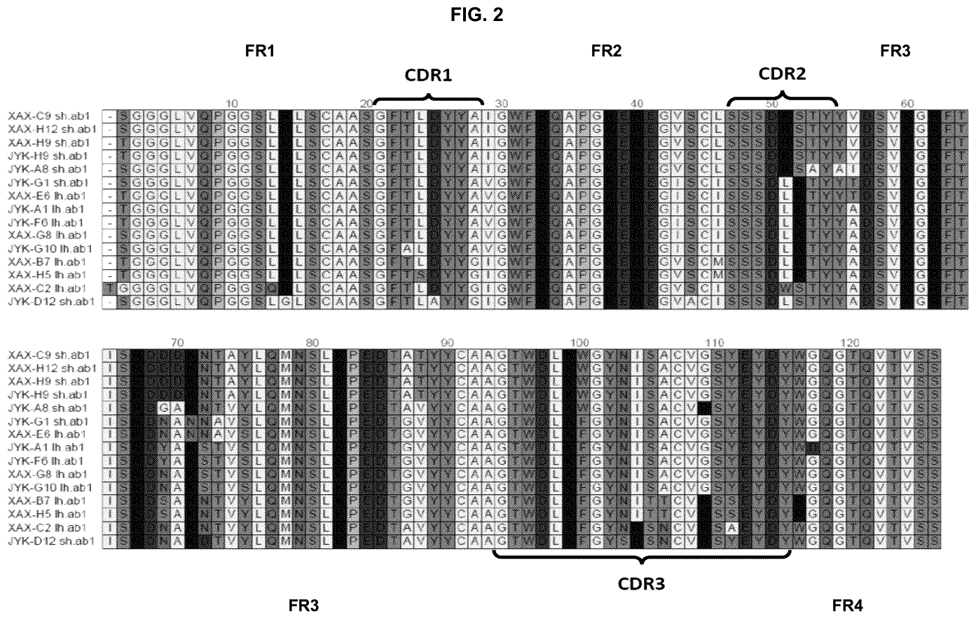

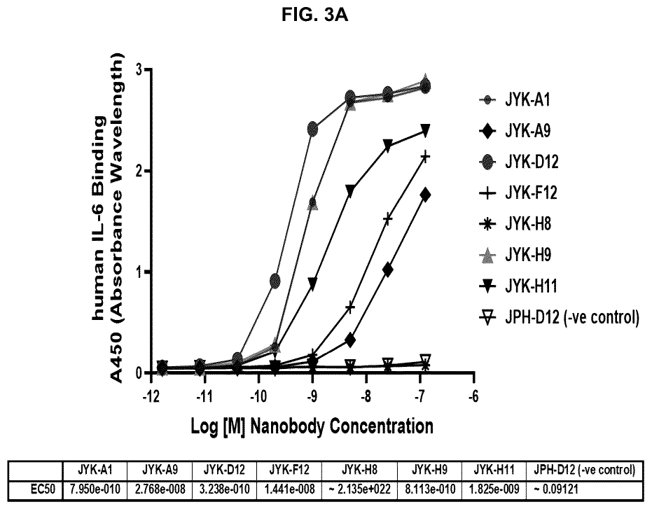

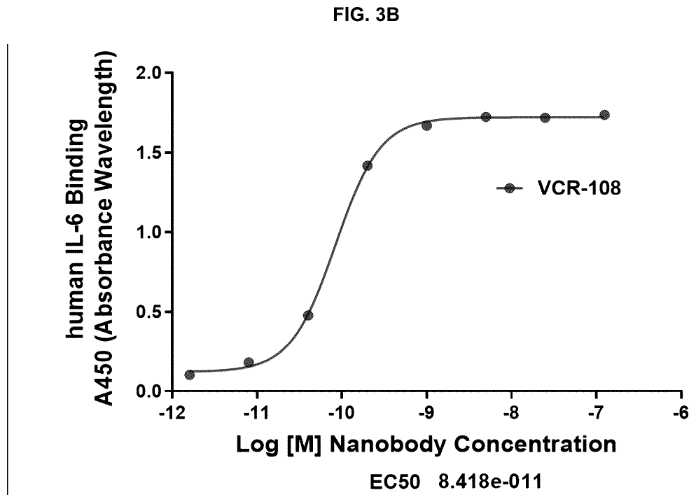

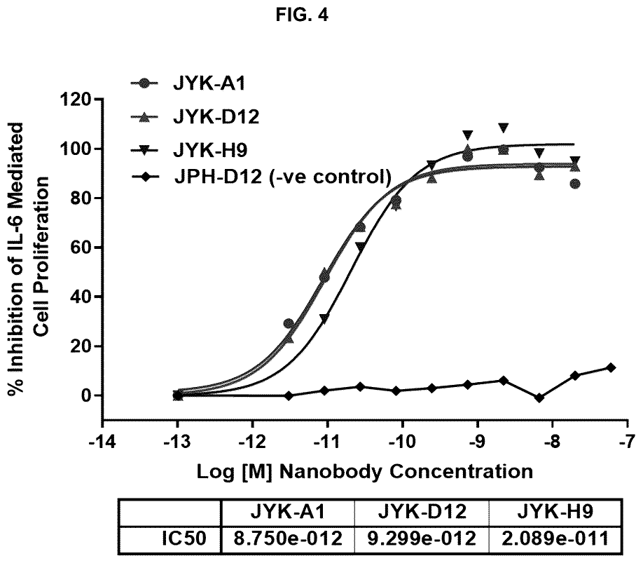

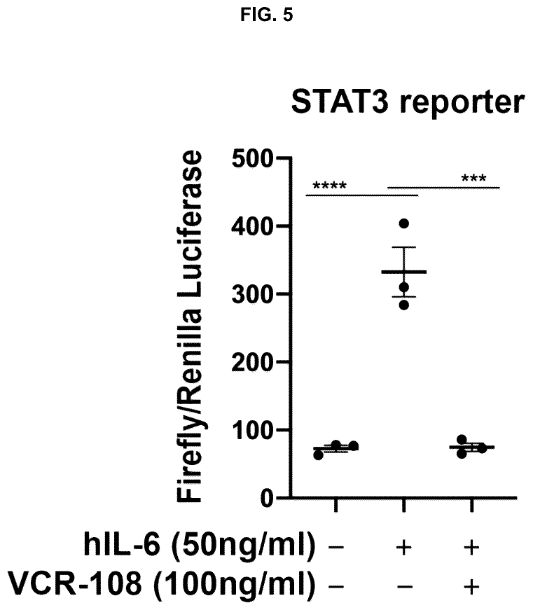

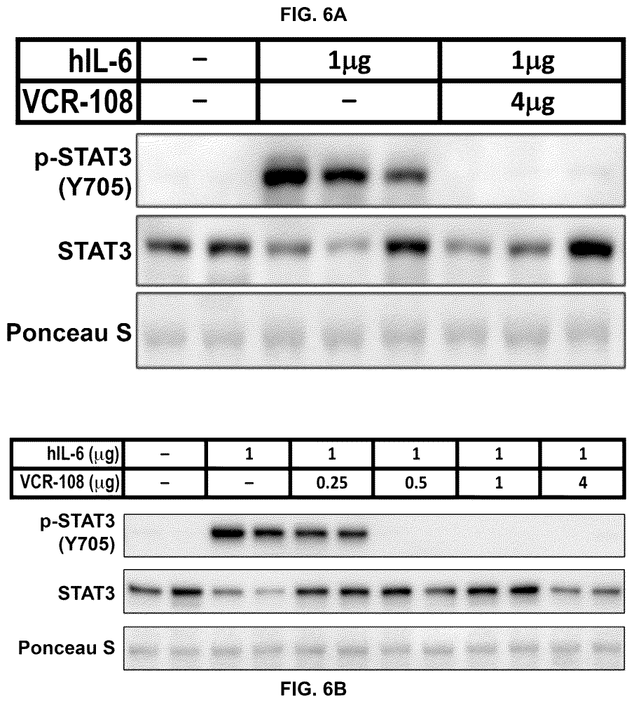

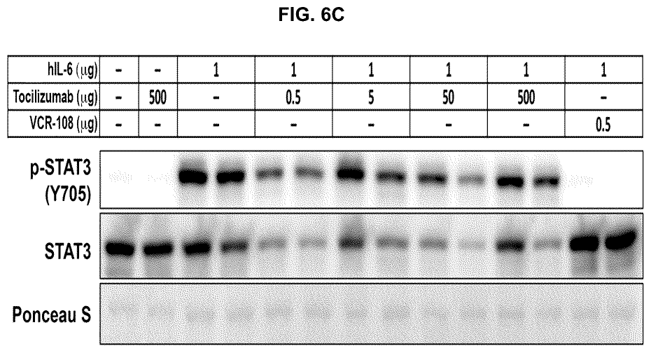

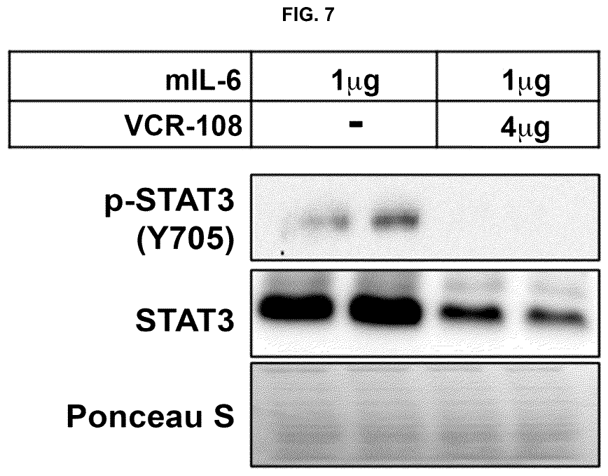

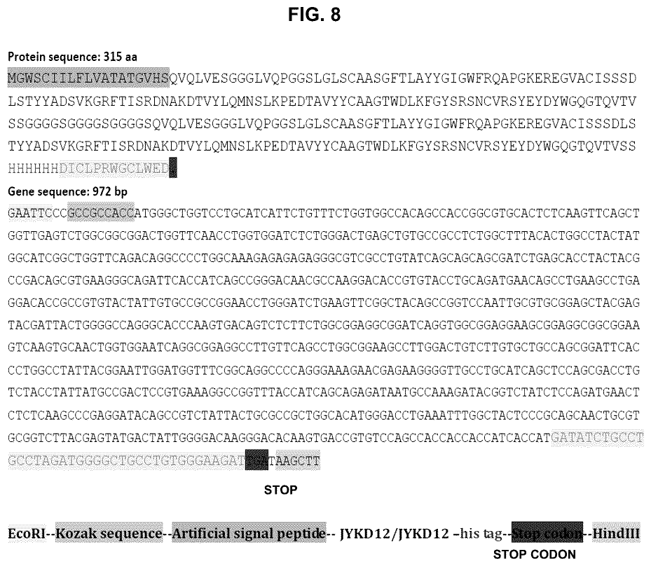

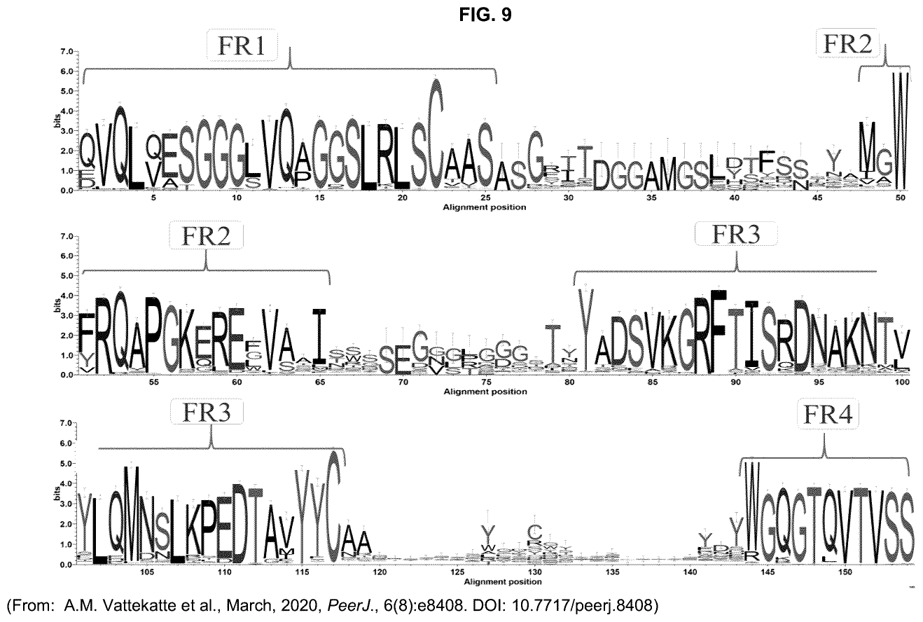

The amino acid sequences of the four framework regions (FR1-FR4) of the IL-6 binding VHHs presented in FIG. 2 are shown in Table 2:

TABLE 2

| VHH | | | | |
|---|---|---|---|---|
| (anti-IL- | | SEQ ID | | SEQ ID |
| 6 VHH) | FR1 | NO: | FR2 | NO: |
| XAX-C9 | -SGGGLVQPGGSLRLSCAAS | 58 | IGWFRQAPGKEREGVSCL | 61 |
| sh.ab1 | | | | |
| XAX-H12 | -SGGGLVQPGGSLRLSCAAS | 58 | IGWFRQAPGKEREGVSCL | 61 |
| sh.ab1 | | | | |
| XAX-H9 | -TGGGLVQPGGSLRLSCAAS | 59 | IGWFRQAPGKEREGVSCL | 61 |
| sh.ab1 | | | | |
| JYK-H9 | -TGGGLVQPGGSLRLSCAAS | 59 | IGWFRQAPGKEREGVSCL | 61 |
| sh.ab1 | | | | |
| JYK-A8 | -SGGGLVQPGGSLRLSCAAS | 58 | IGWFRQAPGKEREGVSCL | 61 |
| sh.ab1 | | | | |
| JYK-G1 | -SGGGLVQPGGSLRLSCAAS | 58 | VGWFRQAPGKEREGISCI | 62 |
| sh.ab1 | | | | |
| XAX-E6 | -TGGGLVQPGGSLRLSCAAS | 59 | VGWFRQAPGKEREGISCI | 62 |
| lh.ab1 | | | | |
| JYK-A1 | -TGGGLVQPGGSLRLSCAAS | 59 | VGWFRQAPGKEREGISCI | 62 |
| lh.ab1 | | | | |
| JYK-F6 | -TGGGLVQPGGSLRLSCAAS | 59 | VGWFRQAPGKEREGISCI | 62 |
| lh.ab1 | | | | |
| XAX-G8 | -SGGGLVQPGGSLRLSCAAS | 58 | VGWFRQAPGKEREGISCI | 62 |
| lh.ab1 | | | | |
| JYK-G10 | -SGGGLVQPGGSLRLSCAAS | 58 | VGWFRQAPGKEREGISCI | 62 |
| lh.ab1 | | | | |
| XAX-B7 | -TGGGLVQPGGSLRLSCAAS | 59 | IGWFRQAPGKEREGVSCM | 63 |
| lh.ab1 | | | | |
| XAX-H5 | -TGGGLVQPGGSLRLSCAAS | 59 | IGWFRQAPGKEREGVSCM | 63 |
| lh.ab1 | | | | |
| XAX-C2 | TGGGGLVQPGGSLRLSCAAS | 60 | IGWFRQAPGKEREGVSCI | 64 |
| lh.ab1 | | | | |
| JYK-D12 | -SGGGLVQPGGSLRLSCAAS | 58 | IGWFRQAPGKEREGVACI | 65 |
| sh.ab1 | | | | |
| VHH | | | | |
| (anti-IL- | | SEQ ID | | SEQ ID |
| 6 VHH) | FR3 | NO: | FR4 | NO: |
| XAX-C9 | YVDSVKGRFTISRDDDKNTA | 66 | WGQGTQVTVSS | 50 |
| sh.ab1 | YLQMNSLKPEDTATYYCAA | | | |
| XAX-H12 | YVDSVKGRFTISRDDDKNTA | 66 | WGQGTQVTVSS | 50 |
| sh.ab1 | YLQMNSLKPEDTATYYCAA | | | |
| XAX-H9 | YVDSVKGRFTISRDDDKNTA | 66 | WGQGTQVTVSS | 50 |
| sh.ab1 | YLQMNSLKPEDTATYYCAA | | | |
| JYK-H9 | YVDSVKGRFTISRDDDKNTA | 66 | WGQGTQVTVSS | 50 |
| sh.ab1 | YLQMNSLKPEDTATYYCAA | | | |
| JYK-A8 | AIDSVKGRFTISRDGAKNTVY | 67 | WGQGTQVTVSS | 50 |
| sh.ab1 | LQMNSLKPEDTAVYYCAA | | | |
| JYK-G1 | YTDSVKGRFTISRDNANNAV | 68 | WGQGTQVTVSS | 50 |
| sh.ab1 | SLQMNSLKPEDTGVYYCAA | | | |
| XAX-E6 | YTDSVKGRFTISRDNANNAV | 68 | WGQGTQVTVSS | 50 |
| lh.ab1 | SLQMNSLKPEDTGVYYCAA | | | |
| JYK-A1 | YADSVKGRFTISRDYAKSTVS | 69 | WDQGTQVTVSS | 74 |
| lh.ab1 | LQMNSLKPEDTGVYYCAA | | | |
| JYK-F6 | YADSVKGRFTISRDYAKSTVS | 69 | WGQGTQVTVSS | 50 |
| lh.ab1 | LQMNSLKPEDTGVYYCAA | | | |
| XAX-G8 | YADSVKGRFTISRDNAKSTVS | 70 | WGQGTQVTVSS | 50 |
| lh.ab1 | LQMNSLKPEDTGVYYCAA | | | |
| JYK-G10 | YADSVKGRFTISRDNAKSTVS | 70 | WGQGTQVTVSS | 50 |
| lh.ab1 | LQMNSLKPEDTGVYYCAA | | | |
| XAX-B7 | YADSVKGRFTISRDSAKNTV | 71 | RGQGTQVTVSS | 75 |
| lh.ab1 | YLQMNSLKPEDTGVYYCAA | | | |
| XAX-H5 | YADSVKGRFTISRDSAKNTV | 71 | RGQGTQVTVSS | 75 |
| lh.ab1 | YLQMNSLKPEDTGVYYCAA | | | |
| XAX-C2 | YADSVKGRFTISRDNAKNTV | 72 | WGQGTQVTVSS | 50 |
| lh.ab1 | YLQMNSLKPEDTAVYYCAA | | | |
| JYK-D12 | YADSVKGRFTISRDNAKDTV | 73 | WGQGTQVTVSS | 50 |
| sh.ab1 | YLQMNSLKPEDTAVYYCAA | | | |

FIGS. 3A and 3B present graphs showing the results of binding assays (ELISAs) performed to determine the binding affinities of representative anti-hIL-6 VHHs and a dimeric anti-hIL-6 VHH, referred to as “VCR-108” herein, to hIL-6 antigen. In FIG. 3A, the anti-hIL-6 VHHs (also referred to as “nanobodies” herein) tested are as follows: the hIL-6-binding VHH JYK-A1 (expression vector JYR-1 containing polynucleotide encoding Trx/JYK-A1/E); the hIL-6-binding VHH JYK-A9 (expression vector JYR-2 containing polynucleotide encoding Trx/JYK-A9/E); the hIL-6-binding VHH JYK-D12 (expression vector JYR-3 containing polynucleotide encoding Trx/JYK-D12/E); the hIL-6-binding VHH JYK-F12 (expression vector JYR-4 containing polynucleotide encoding Trx/JYK-F12/E); the hIL-6-binding VHH JYK-H8 (expression vector JYR-5 containing polynucleotide encoding Trx/JYK-H8/E); the hIL-6-binding VHH JYK-H9 (expression vector JYR-6 containing polynucleotide encoding Trx/JYK-H9/E); and the hIL-6-binding VHH JYK-H11 (expression vector JYR-8 containing polynucleotide encoding Trx/JYK-H11/E). JPH-D12 denotes the negative control (expression vector JWE-8 containing polynucleotide encoding Trx/JPH-D12/E). The EC50 values for each of the hIL-6-binding VHHs tested are provided below the graph. Briefly, the EC50 value for JYK-A1 was 7.950e-010; the EC50 value for JYK-A9 was 2.768e-008; the EC50 value for JYK-D12 was 3.238e-010; the EC50 value for JYK-F12 was 1.441e-008; the EC50 value for JYK-H8 was ˜2.135e+022; the EC50 value for JYK-H9 was 8.113e-010; the EC50 value for JYK-H11 was 1.825e-009; and the EC50 for the JPH-D12 negative control was ˜0.09121. The graph in FIG. 3B shows the binding of a homodimer comprising the anti-hIL-6-binding VHH JYK-D12 (“VCR-108”) to hIL-6. FIGS. 3A and 3B demonstrate and establish that the anti-hIL-6 VHH nanobodies and an anti-hIL-6 VHH dimer exhibit potent and specific binding to hIL-6.

FIG. 4 presents a graph showing the results of cell proliferation assays performed to determine the percent inhibition of IL-6-mediated cell proliferation. The representative hIL-6-binding VHH antibodies (Nanobodies) tested in FIG. 4 include the anti-hIL6 VHHs JYK-A1, JYK-D12, JYK-H9 and the negative control JPH-D12. The IC50 value for JYK-A1 was 8.750e-012; the IC50 value for JYK-D12 was 9.299e-012; and the IC50 value for JYK-H9 was 2.089e-011.

FIG. 5 presents a dot plot showing the results of in vitro neutralization assays in which hepatic JAK-STAT signaling was assessed. A representative hIL-6-binding VHH antibody (100 ng) was found to neutralize (abolish) hIL-6 induced JAK-STAT signaling in HEK293 cells. The representative anti-hIL-6 VHH antibody in the experiments was in the form of a recombinant homodimer in which the two anti-hIL-6 VHH antibody components (JKY-D12) were linked by a short spacer or linker, e.g., as described in Example 7.

FIGS. 6A-6C present Western blots showing the results of in vivo experiments in which JAK-STAT signaling, as assessed by STAT phosphorylation status, was abolished by a representative anti-hTL-6 VHH antibody following injection with hIL-6 into animals. The representative anti-hTL-6 VHH antibody in the experiments was in the form of a recombinant homodimer in which the two anti-hTL-6 VHH antibody components (JKY-D12) were linked by a short spacer or linker.

FIG. 7 presents a Western blot showing that a representative anti-hIL-6 VHH antibody as described herein cross-reacts with mouse IL6 in vivo.

FIG. 8 presents the amino acid (polypeptide) and encoding polynucleotide sequences of a homodimer of the JYK-D12 anti-hIL-6 VHH antibody described herein. The JYK-D12/JYK-D12 homodimer was recombinantly produced and expressed in mammalian cells (Expi293F cells) using the mammalian expression plasmid vector pcDN3.4. Also shown in FIG. 8 is a linear depiction of the expression plasmid encoding the JYK-D12 anti-hTL-6 VHH antibody homodimer. The expression plasmid includes the following components, from left to right: EcoR1 restriction enzyme site; Kozak sequence; artificial signal peptide; dimer of JYK-D12 anti-hIL-6 VHH antibody; histidine tag (his-tag); stop codon; and HindIII restriction enzyme site. FIG. 8 discloses SEQ ID NOS 91 and 92, respectively, in order of appearance.

FIG. 9 depicts a sequence logo representation of a multiple sequence alignment of a complete dataset of VHH sequences showing sequence conservation among VHH framework regions (FRs), (See, A. M. Vattekatte et al., March, 2020, PeerJ., 6(8):e8408. DOI: 10.7717/peerj.8408, incorporated by reference herein). The relative frequency of amino acids at each position shown in FIG. 9 is shown as a sequence logo. In many cases, the residues may have similar chemical properties. The residue positions are not in accordance with the numbering systems, as sequence alignment creates a longer length than canonical VH. As set forth in Vattekatte et al., for the analysis of different VHH protein sequences or the VHH sequences within a VHH protein family, the amino acid sequence characteristics were determined using a multiple sequence alignment (MSA). Such an alignment was generated with a VHH sequence dataset using Clustal Omega. FIG. 9 shows the analysis of a MSA represented as a sequence logo, where residue conservation at each position was calculated as information content (bits). FR positions appear as conserved sequence blocks evidenced by high bit scores. The interspersed CDRs, which have greater sequence variability, have less information content in terms of bits. The analysis was performed for each genus (camel, alpaca, llama) of VHH. Despite the divergence of species, sequence conservation shows similar conservation for all the different regions with no significant differences. In general, VHH sequences have a median length value of 123 amino acids (aa) with a minimum and maximum length of 109 aa and 137 aa, respectively. The amino acid length distribution in different regions of VHHs shows diversity in CDR lengths, especially in CDR3. The median values for CDR lengths are 8 aa, 8 aa and 16 aa for CDR1, CDR2 and CDR3, respectively. The average length of CDR3 in VHH is greater than that of conventional human or mouse immunoglobulin VH sequences. VHH FRs are not of an absolute invariant length, e.g., FR1 may be 21-29 (e.g., 25) aa in length, FR2 may be 14-22 (e.g., 18) aa in length, FR3 may be 33-41 (e.g., 37) aa in length, and FR4 may be 10-13 (e.g., 11) aa in length, with differences of 2 to 3, or 2 to 4, residues for each of the FR lengths. These differences in FR length are considered so that bias is not introduced into the sequence conservation analysis. Without wishing to be bound by theory, the pairwise sequence identity between sequences in the dataset has a median value of 62% and is always above 35%. In general, the variability of amino acids is not constant in the FR and CDR regions of a VHH. FRs are more conserved, with sequence identity 84, 72, 81 and 90% (median values) for FR1, FR2, FR3 and FR4, respectively among VHHs. For CDRs, low sequence identities are observed (below 30%); in general, sequence identity in CDR3 is the lowest with 18%, followed by CDR2 with 25% and CDR1 with 28%. (See, A. M. Vattekatte et al., March, 2020, PeerJ., 6(8):e8408. DOI: 10.7717/peerj.8408).

### DETAILED DESCRIPTION OF THE DISCLOSURE

Described herein are single domain antibody (sdAb) binding molecules, which are comprised of the heavy chain variable (V_{H}) region of heavy-chain-only antibodies (Abs), that specifically bind to interleukin 6 (IL-6) polypeptide, in particular, human interleukin 6 (hIL-6) polypeptide, which is a cytokine that is produced by white blood cells, including monocytes, macrophages, and lymphocyte subsets, as well as fibroblasts, keratinocytes, astrocytes endothelial cells, and adipose tissue cells (adipocytes), under or in response to various physiological conditions.

Single-domain antibodies (camelid single-domain antibodies or nanobodies) are called VHHs as they derive from the V_{H} region of a class of heavy-chain-only antibodies. The anti-hIL-6 VHHs were produced form immunized camelid animals (alpacas) and were selected for their ability to specifically bind to hIL-6 and, in many cases, to neutralize hIL-6 and thus reduce the adverse effects and functional activity of the hIL-6 protein. The anti-hIL-6 VHHs as described herein are hIL-6-binding polypeptides comprising hypervariable variable regions (CDRs) within framework (FR) regions. In general, the FRs of the anti-hIL-6 VHHs are typically highly similar in amino acid sequence, or differ by, conservative amino acid substitutions at certain positions of the FR sequences, among different anti-hIL-6 VHHs or families of anti-hIL-6 VHHs.

The anti-hIL-6 VHHs as described provide advantageous properties, particularly for therapeutic use. By way of nonlimiting example, these sdAb molecules are small proteins (e.g. about 14 Kda), thus facilitating the cloning of their encoding polynucleotides. The anti-hIL-6 VHHs can be functionally expressed at high levels, are stable to extreme pH and high temperatures over time, and function well in multimeric forms, e.g., dimers and other multimers, to provide improved binding and neutralization properties and therapeutic efficacy.

The anti-hIL-6 VHHs are employed as therapeutic agents for the treatment and prevention of IL-6-mediated and associated disorders, conditions, or diseases as described herein. It will be understood that the terms “anti-IL-6 VHH antibody,” “anti-IL-6 VHH polypeptide,” “anti-IL-6 VHH antibody polypeptide,” “anti-IL-6 VHH,” and “IL-6 VHH” are used interchangeably herein.

### Interleukin 6 (IL-6)

Interleukin-6 (IL-6), namely, human IL-6 (hIL-6), is a pleiotropic pro-inflammatory cytokine having a number of physiological functions including regulation of immune cell proliferation and differentiation. The deregulation or dysregulation of IL-6 is associated with chronic inflammation, and multifactorial auto-immune disorders. In general, the IL-6 protein cytokine mediates its biological roles through a hexameric complex composed of IL-6 itself, its receptor IL-6R, and glycoprotein 130 (IL-6/IL-6R/gp130). This complex, in turn, activates different signaling mechanisms (classical and trans-signaling) that carry out various biochemical functions. The trans-signaling mechanism activates certain pathological routes, such as JAK/STAT3, Ras/MAPK, PI3K-PB/Akt, and the regulation of CD4+ T cells and VEGF levels, which are involved in, or cause, cancer, multiple sclerosis, rheumatoid arthritis, anemia, inflammatory bowel disease, Crohn's disease, and Alzheimer's disease. Involvement of IL-6 in pathophysiology of complex diseases makes it an important target for the treatment of these diseases. In particular, aberrant or dysregulated IL-6 signaling is associated with inflammatory and lymphoproliferative disorders and diseases, such as, without limitation, autoimmune diseases, e.g., rheumatoid arthritis (e.g., adult rheumatoid arthritis and juvenile idiopathic arthritis), Castleman disease, secondary amyloidosis, polymyalgia rheumatic, adult onset Still's disease, polymyositis, systemic sclerosis, large vessel vasculitis lupus erythematosus, Crohn's disease, irritable bowel disease/disorder (IBD), Sjogren's syndrome; steroid refractory Graft versus Host Disease in transplantation; type 2 diabetes, obesity, and schizophrenia, as well as cancers such as cholangiocarcinoma, ovarian cancer, and multiple myeloma.

While different classes of therapeutic agents have been developed to target components of the IL-6 signaling pathway, such agents that target IL-6 signaling have raised questions about their utility and appropriate benefit-risk profile in treating certain diseases and patient populations. The anti-IL-6 VHH antibodies described herein offer new and efficacious therapeutics for the treatment of diseases and disorders associated with IL-6 production/overproduction and signaling dysregulation, for example, in blocking the interaction of IL-6 and its receptor (IL-6R). IL-6 is historically also known as BSF-2, BSF2, CDF, HGF, HSF, IFN-beta-2 and IFNB2.

### IL-6 Biology

Human IL-6 is a four-helical polypeptide cytokine of 184 amino acids that may be secreted by many cell types upon appropriate stimulation during infection, e.g., virus infection, such as SARS-Covid19, Adult Respiratory Distress Syndrome (ARDS), inflammation, or cancer. By way of example, IL-6 is secreted by monocytes and macrophages following binding of Toll-like receptors (TLRs) by cognate ligands, for example, lipopolysaccharides (LPS); by fibroblasts, keratinocytes, astrocytes and endothelial cells after stimulation by IL-1 cytokine; and by subsets of activated B cells and T cells and microglial cells after viral infection. IL-6 is important for regulating B cell and T cell responses and for coordinating the activity of the innate and the adaptive immune response systems. IL-6 is also needed for liver regeneration.

Under normal conditions (in healthy or non-diseased subjects or individuals), the concentration of IL-6 in the circulation is around 1-5 picograms per milliliter (pg/ml); however, under pathological conditions or disease states, the concentrations of IL-6 in serum can increase into the nanogram per milliliter (ng/ml) range. IL-6 is strongly induced during most, if not all, inflammatory processes, infections, e.g., virus infection, and cancer. In sepsis, IL-6 levels of several micrograms per milliliter (g/ml) in serum have been reported. In the brain, high IL-6 levels can lead to astrocytosis and neurodegeneration.

The IL-6 polypeptide binds to the IL-6 receptor (IL-6R or IL-6R subunit-α), which is an 80 kDa receptor devoid of signaling capacity. The complex of IL-6 and IL-6R (IL-6/IL-6R) binds to a second membrane protein, glycoprotein 130 (gp130; also known as IL-6R subunit-r$), which dimerizes and initiates intracellular signaling. While gp130 is expressed on all cells, IL-6R is found on only a few cells, such as hepatocytes, as well as some leukocytes and epithelial cells. Because IL-6 exhibits measurable affinity only for IL-6R but not for gp130, cells that express gp130 but not IL-6R are not responsive to IL-6 per se. The gp130 protein has been shown to act as a signaling receptor for additional cytokines, including IL-11, oncostatin M (OSM), ciliary neurotrophic factor (CNTF), cardiotrophin 1 (CT1), leukemia inhibitory factor (LIF) and the cardiotrophin-like cytokine factor 1 (CLCF1), which, together with IL-6, form the IL-6 family of cytokines. In addition, gp130 is a component of the heterodimeric receptor complexes for some heterodimeric IL-12 family members, including IL-27. (See, e.g., Garbers, C. et al., 2018, Nature, Vol. 17, pages 395-412).

IL-6R can exist in a soluble form (sIL-6R) that binds to IL-6 with an affinity similar to that of membrane-bound IL-6R. However, unlike other soluble receptors that compete with membrane-bound receptors for binding to the cognate ligand and therefore act as antagonists, the complex of sIL-6R/IL-6 binds to gp130 and induces dimerization, which results in intracellular signaling. Of note, cells that do not express the IL-6R and thus are not responsive to IL-6, can be stimulated by the complex of sIL-6R/IL-6—this process is termed IL-6 trans-signaling. IL-6 trans-signaling can be selectively blocked by the soluble form of gp130 (sgp130Fc), which is dimerized by a human immunoglobulin IgG1-Fc, without affecting IL-6 signaling via the membrane-bound IL-6R.

IL-6 trans-presentation was recently discovered to be a third mode of IL-6 signaling that occurs in the context of the antigen-specific interaction of a dendritic cell (DC), which provides the IL-6 signal, and a T cell, which receives it, resulting in the commitment of the T cell to a highly tissue-destructive phenotype. In order to develop clinical signs of experimental autoimmune encephalomyelitis (EAE), which is a model for human multiple sclerosis, murine DCs simultaneously express IL-6 and the IL-6R. Without intending to be bound by theory, IL-6 binds to the IL-6R within intracellular compartments of the DC and is transported to the plasma membrane. The DC then presents the membrane-bound IL-6/IL-6R complex from cell to cell to cognate, interacting T cells, which can sense and respond to the DC-derived IL-6/IL-6R complex via the T cells' own gp130. This results in phosphorylation of signal transducer and activator of transcription 3 (STAT3) in the T cell and, subsequently, the induction of a pathogenic effector T cell program. In general. antibodies directed against IL-6 block classic signaling of IL-6 via the membrane-bound IL-6R, and trans-signaling of IL-6 via the sIL-6R. Antibodies directed against IL-6R block all types of IL-6 signaling. (Garbers, C. et al., 2018, Nature, Vol. 17, pages 395-412).

Intracellular signaling occurs mainly via JAK1, which is constitutively bound to the cytoplasmic portion of gp130 and is activated by gp130 dimerization. JAK1 phosphorylates cytoplasmic tyrosine residues of gp130, leading to the activation of the RAS-MAPK-PI3K (RAS-mitogen-activated protein kinase-phosphoinositide 3-kinase) pathway and to the phosphorylation and activation of STAT1 and STAT3. STAT1 and STAT3 homodimerize or heterodimerize, translocate to the nucleus, and serve as transcription factors to induce the activation of gp130 target genes such as BCL2, BIRC5 (also known as survivin), MYC, NOTCH1, cyclins and several matrix metalloproteinases. In patients, somatic mutations in hepatocyte gp130 have been detected in 60% of inflammatory hepatocellular adenomas. These mutations activated gp130 in the absence of IL-6. Phosphorylation and activation of transcriptional coactivator YES-associated protein 1 (YAP1) by the SRC family kinases, SRC and YES, are also triggered via gp130 and contribute to the development of colon cancer. (Garbers, C et al., 2018, Nature, Vol. 17, pages 395-412; Jones, S. A. and Jenkins, B. J., 2018, Nature Reviews Immunol., Vol. 18, pages 773-789).

### IL-6 in Normal Physiology and in Disease

During the past 20 years, research studies have demonstrated that IL-6 and its activity are associated with or involved in a wide repertoire of biological functions, thus establishing IL-6 as a pleiotropic cytokine that plays a major role in health and in disease. In general, IL-6 fulfils homeostatic functions, which include immune cell proliferation and differentiation under normal or healthy conditions, as well as metabolic functions and pro-inflammatory actions due to dysregulated activity. Therefore, the homeostatic functions of TL-6 are optimally spared when targeting IL-6 in order to avoid serious long-term side effects. By contrast, the pro-inflammatory activities of IL-6, which often correlate with increased and/or prolonged protein expression and activity of the cytokine, are the targets of inhibition to efficiently and effectively control diseases associated with IL-6 function.

Under normal circumstances, IL-6 is a physiological regulator of energy metabolism in the liver and in skeletal muscle, supporting insulin in eliminating free glucose. Moreover, IL-6 signaling is an essential promoter of energy expenditure. The net effect of IL-6 derived from adipose tissue in the steady state is believed to be catabolic because TL-6 drives fatty acid oxidation. Accordingly, anabolic side effects, including increased serum triglyceride and cholesterol levels and increased body weight, are commonly reported with the use of certain anti-IL-6 therapies. Conversely, increased TL-6 serum levels have been described in patients with obesity, and weight reduction is accompanied by a reduction in IL-6 serum levels. In addition, IL-6 and CRP levels were found to be elevated in patients with type 2 diabetes than in controls; therefore, these levels are considered to be a risk factor for developing obesity. IL-6 can be released from macrophages in the adipose tissue, and it has been shown that increased IL-6 levels found in patients with type 2 diabetes are related to fat mass. In addition to data obtained from human studies, studies conducted in mouse models using different genetically modified mouse strains (e.g., Il6^{-/-} mice) show that these mice develop mature-onset obesity, with an increase of around 50% weight in fat pad mass, and decreased glucose tolerance. Classic IL-6 signaling in T cells is critical for protection from insulin resistance in the early stages of obesity, which is switched to trans-signaling at later time point. IL-6 trans-signaling is the molecular pathway that triggers the recruitment of macrophages into adipose tissue where IL-6 stimulates polarization of macrophages to an M2 state (highly phagocytic, anti-inflammatory cytokine secretion) and proliferation of M2 macrophages. (Garbers, C. et al., 2018, Nature, Vol. 17, pages 395-412).

IL-6 is also considered to be an important regulator of bone homeostasis. Osteoporosis, a disease characterized by bone weakness that results in an increased risk of bone fractures, is highly prevalent in elderly people and postmenopausal women. Mice that overexpress IL-6 show osteopenia due to a disturbed osteoclast-osteoblast balance with decreased numbers of osteoblasts and increased numbers of osteoclasts. Estrogens suppress IL-6 production by bone marrow stromal cells and osteoblasts, and estrogen deficiency after menopause results in elevated IL-6 levels and bone loss. In general, IL-6 induces osteoblast expression of receptor activator of nuclear factor-κB ligand (RANKL; also known as TNFSF11), an important factor for osteoclast differentiation; however, only osteoblasts but not osteoclasts express IL-6R. (Garbers, C. et al., 2018, Nature, Vol. 17, pages 395-412).

### Acute Phase Response

The acute phase response is mediated by the innate immune system and provides a nonspecific and powerful mechanism that protects from infections (, e.g., virus infection, such as SARS-Covid19) and pathogens, as well as tissue damage. Activation of the acute phase response results in the secretion of a variety of proteins from the liver, including C-reactive protein (CRP), serum amyloid A (SAA), fibrinogen and haptoglobin. While CRP stimulates phagocytosis, IL-6 enhances the production of the clotting factor fibrinogen. When the liver secretes these acute phase reactants, other proteins like albumin and transferrin are necessarily secreted in lesser quantities Mechanistically, such acute phase proteins are induced mainly by IL-6, and also by cytokines such as IL-1β or TNF. The production of acute phase reactants causes the onset of fever, high glucocorticoid levels, activation of complement pathways, and activation of coagulation pathways. A high erythrocyte sedimentation rate (ESR) is another manifestation that may indicate inflammation. By way of example, patients who were administered a therapeutic blockade of all modes of IL-6 signaling experienced bacterial infections as the most common serious adverse event.

### IL-6 in Pathophysiological States

Dysregulated IL-6 can contribute to initiating and perpetuating tissue damage in autoimmunity and chronic inflammation in view of its activity as a growth factor for many hematopoietic cells and through induction of pathogenic adaptive immune cells. The efficacy of therapeutic interventions neutralizing IL-6 or interfering with its signaling supports a major pathogenic role of IL-6 in Castleman disease, rheumatoid arthritis (including polyarticular and systemic juvenile idiopathic

A number of non-neoplastic hematological conditions, including Erdheim-Chester disease and Castleman disease, have been identified as being due to dysregulation of IL-6 production. For example, Castleman disease is driven by exaggerated production of IL-6 either by human herpesvirus 8 (HHV-8)-infected cells or, in HHV-8-negative cases (about one-third), by unknown cellular sources. Viral IL-6 (vIL-6) is encoded by HHV8 and might contribute to the expansion of B lymphocytes via IL-6R-independent engagement of gp130. Therefore, to date, therapeutic interventions with certain antibodies against IL-6 or IL-6R have been approved only for HHV-8 negative cases of Castleman disease. Because the ability to develop Castleman disease-like symptoms by vIL-6 in mice required the presence of endogenous IL-6, the use of anti-IL-6 VHH treatment and/or anti-IL-6R therapy may be beneficial for patients who are positive for HHV-8. Moreover, IL-6 has been implicated in the regulation of hepcidin, which is a liver-derived antimicrobial peptide and a key regulator of body iron homeostasis. Increased hepcidin levels induced by IL-6 cause anemia in subjects by downregulating the expression of the iron exporter ferroportin. Treatment of subjects (patients) with Castleman disease by administering an anti-IL-6 VHH as described herein to block IL-6 activity may advantageously reduce hepcidin levels in serum and normalize iron-related parameters in treated subjects (patients). (Garbers, C. et al., 2018, Nature, Vol. 17, pages 395-412).

### IL-6 and Autoimmunity and Chronic Inflammation

IL-6 targeted interventional therapies such as the administration of the anti-IL-6 VHH antibodies described herein are advantageous for blocking, inhibiting, attenuating, reducing, ameliorating, alleviating, and/or eliminating the pathogenic activity of IL-6 in rheumatoid arthritis and juvenile idiopathic arthritis, as well as in giant cell arteritis. The treatment of the autoimmune disease neuromyelitis optica (NMO), also known as neuromyelitis optica spectrum disorder or Devic's disease, is also provided by the anti-IL-6 VHH antibodies as described herein. NMO is a central nervous system disorder that primarily affects the nerves of the eye (optic neuritis) and the spinal cord (myelitis). In NMO, autoantibodies against aquaporin 4 (AQP4) lead to the functional impairment and destruction of a subset of astrocytes, and secondary demyelination in the spinal cord and in distinct parts of the central nervous system. Without wishing to be bound by theory, the pathogenic role of IL-6 in NMO may involve both its promotion of responses from T helper 17 (T_{H}17) cells against AQP4 and the expansion of plasmablasts that produce antibodies directed against AQP4. Distinct modes of IL-6 signaling in various cells are associated with distinct biological outcomes. The goals of treatment and/or IL-6-directed intervention using the anti-IL-6 VHH antibodies may involve targeting the pathogenic processes of inflammation and/or autoimmune diseases such as rheumatoid arthritis or in NMO that are associated with different IL-6 signaling modalities, or affecting the activation of B cells versus the priming of pathogenic T cells, in order to block, inhibit or subdue the inflammatory cascade spearheaded by IL-6 activity. (Garbers, C. et al., 2018, Nature, Vol. 17, pages 395-412).

### IL-6 and T Cells

For adaptive immune responses in host defense, T_{H}1 cells produce interferon-γ (IFNγ) and protect against intracellular pathogens, while T_{H}2 cells produce IL-4 and orchestrate host defense against parasites. In general, IL-6 was associated with the induction of T_{H}2 responses rather than with the induction of T_{H}1 responses. However, it has been established that IL-6 is an essential differentiation factor for a subset of TH cells, termed T_{H}17 cells, based on the cytokine IL-17 that is produced by these cells.

Because differentiation factors, as well as distinct and mutually exclusive transcriptional networks, have been defined for T_{H}1, T_{H}2 and T_{H}17 cells, these CD4⁺ T_{H} cell subsets have been considered as T cell ‘lineages’. However, T_{H}17 cells exhibit some plasticity and can co-produce other cytokines including IL-22, which is strongly induced by IL-23. As T_{H}17 cells express the largest amounts of the IL-23 receptor (IL-23R), T_{H}17 cells produce IL-22 in response to IL-23. T_{H}22 cells can be generated from naive CD4⁺ T cells in response to IL-6 and TNT and have a role in host defense at epithelial barriers, including the skin and the gut, where IL-22 is an inducer of antibacterial peptides. T_{H}17 cells are highly responsive to IL-Aβ, a growth factor for T_{H}17 cells that may skew them toward a more inflammatory phenotype by suppressing T_{H}17-intrinsic IL-10 production in mice and humans.

Naive T cells express IL-6Rα and respond to IL-6 alone, the complex of IL-6/sIL-6R, or a fusion protein of IL-6 and sIL-6R. Once T cells are activated, IL-6Rα is shed from the surface of both conventional T cells and FOXP3⁺ T_{reg} cells, most probably by the ADAM17 metalloproteinase. Consequently, activated T cells still respond to IL6-sIL6R (hyper-IL-6), but they become resistant to IL-6 alone. T cells have been shown to respond to IL-6 via trans-presentation, a mode of IL-6 signaling that requires close proximity between cells, for example, during a cognate DC-T cell interaction, and the expression of gp130, but not IL-6Rα, by the receiving cell. (Garbers, C. et al., 2018, Nature, Vol. 17, pages 395-412). The IL-6 signal conveyed by IL-6 trans-presentation when the IL-6 signal is synchronized with the T cell receptor (TCR) signal may be responded to by T cells in a different manner, compared with IL-6 classic signaling. By way of example, IL-6 classic signaling is sufficient to suppress the induction of FOXP3 in naive T cells, but it is not sufficient to induce encephalitogenic T_{H}17 cells.

In the context of IL-6 biology and its complexity, a number of different parameters or circumstances may be involved in the interplay of the IL-6 cytokine and cells reactive to it, thereby affecting whether IL-6 is associated with a disease or pathology, or a normal state. By way of example, soluble ambient IL-6, which is available during massive inflammation when the IL-6 buffer system is saturated, is not misinterpreted by T cells as a signal to become tissue destructive. In addition, in local niches where the systemic IL-6 buffer system might not be effective, ambient IL-6 is sufficient to suppress the induction of FOXP3⁺ T_{reg} cells, but does not result in tissue-destructive T_{H}17 cells. In another case, highly pro-inflammatory T_{H}17 cells, which are required for host protection in response to certain pathogens, but which induce massive immune pathology in autoimmune reactions, are primed only upon IL-6 trans-presentation. This mode of IL-6 signaling may not only synchronize the IL-6 signal with the cognate antigen signal, but also may uncouple the IL-6 signal from the systemic IL-6 buffer system. Thus, therapeutic interventions targeting IL-6 may be optimally designed to block IL-6 trans-presentation in order to blunt auto-destructive T cell responses. As such, diseases that rely on antigen-specific T cell responses, e.g., multiple sclerosis, may be efficiently treated by blocking or preventing IL-6 trans-presentation. (Garbers, C. et al., 2018, Nature, Vol. 17, pages 395-412).

### IL-6 and T_{H}17 Cells in Autoimmunity

T_{H}17 cells are major players in inducing tissue damage in the course of a variety of autoimmune and chronic inflammatory disorders. IL-6 serves as a non-redundant differentiation factor of T_{H}17 cells and, as such, is associated with the disease process in EAE (model of multiple sclerosis), in models of rheumatoid arthritis, and in psoriasis. IL-17 is also produced by cells other than T_{H}17 cells. Thus, the importance of IL-6 is less clear in disease models involving these other cellular sources of IL-17. For example, invariant natural killer T cells (iNKT cells), γδ T cells, and type 3 innate lymphoid cells also produce IL-17. However, in contrast to T_{H}17 cells, their development is independent of IL-6. In models of inflammatory bowel disease (IBD), IL-6 is an important pro-inflammatory as well as anti-inflammatory factor. While IL-6 induces intestinal regeneration, it also can induce pro-inflammatory responses via IL-6 trans-signaling. The role of IL-6 in models of IBD is complex and involves more than the capacity of IL-6 to contribute to the induction of T_{H}17 cells. In lupus erythematosus models, IL-6 may have a direct pro-proliferative role on B cells, which may result in increased levels of anti-double-stranded DNA antibody titers in patients afflicted with this autoimmune disorder. (Garbers, C. et al., 2018, Nature, Vol. 17, pages 395-412).

### IL-6 and T_{H}17 Cells in Host Defense

The physiological function of T_{H}17 cells might serve as host defense against specific pathogens that are not efficiently addressed by T_{H}1 and T_{H}2 cellular responses. For example, some infections with extracellular bacteria (e.g., Klebsiella) and fungal infections (e.g., Candida), require T_{H}17 responses to be efficiently cleared.

Host defense against a variety of viral infections requires the mounting of neutralizing antibody responses. In addition to its role as a growth factor for B cells, IL-6 also promotes the development of T follicular helper (T_{FH}) cells, which are essential for the induction of germinal centers, where somatic hypermutation and class-switch recombination in B cells occur to mount high-affinity antibodies. The T_{FH} cell developmental pathway (which includes B cell lymphoma 6 protein (BCL-6) as a major transcription factor) is believed to be distinct from the differentiation pathways of other TH cell subsets. T_{FH} cell differentiation requires cognate interaction of naive T cells with DCs at the T cell-B cell boundary in secondary lymphoid tissues, followed by T cell-B cell interaction (and presentation of protein antigens by B cells to T cells in the germinal center light zone). As shown in mice, the IL-6 and IL-21 cytokines are essential for the differentiation of T_{FH} cells; the frequency of T_{FH} cells is reduced in the absence of either cytokine, and T_{FH} cells are essentially absent when both cytokines are absent. These studies also identified B cells and T cells as the cellular source of IL-6 and IL-21, respectively. B cell-derived IL-6 was necessary and sufficient to induce IL-21 in T cells, which induced T_{FH} cell development in an autocrine manner. Other sources of IL-6 appear to be important for the delayed generation of T_{FH} cells and protective antibody responses in chronic viral infections. For example, follicular dendritic cells (FDCs) provide IL-6 for the differentiation of T_{FH} cells in lymphocytic choriomeningitis virus (LCMV) infection. IL-6 seems to overcome the dysfunctional state of virus-specific CD4⁺ T cells in chronic LCMV infection by inducing a T_{FH} transcriptional program in CD4⁺ T cells, which leads to the production of protective anti-LCMV antibodies and clearance of the pathogen. IL-21 cannot compensate for the loss of IL-6 in the induction of T_{FH} cells in later stages of viral infections. IL-6 may directly induce BCL6 in CD4⁺ T cells via STAT1, while IL-6-induced STAT3 activation protects CD4⁺ T cells from alternative fates, particularly the T_{H}1 transcriptional program, by downregulating IL-2Rα. (Garbers, C. et al., 2018, Nature, Vol. 17, pages 395-412).

### Therapeutic Strategies to Target IL-6

As reported by Garbers et al. (Id.), the mode by which cytokines and growth factors elicit their biological activities in cells can be pictured as a funnel. Hundreds of secreted signaling proteins outside of the cells, which often can form homodimers and heterodimers that further increase the number of different ligands, bind to their membrane-bound receptors on target cells. Specific receptors for individual ligands exist; however, many receptors (especially signal-transducing receptors) are shared by several ligands. Therefore, the number of receptors is smaller than the number of extant signaling proteins. After the formation of the signaling complex within the plasma membrane, an even smaller number of signaling pathways exist that communicate the signal from the membrane to the nucleus of the cell.

Cytokine signaling envisioned as a funnel provides insights for therapeutic intervention. The direct blockade of an individual cytokine, for example, IL-6, enables targeting of a single signaling entity and does not interfere with all the other cytokines and growth factors that use parts of the same signaling cascade. Targeting a receptor, such as IL-6R or gp130, reduces specificity, because other cytokines that use the same receptor, even in a different combination with a second receptor, would also be blocked. The targeting of a kinase or transcription factor represents a most nonspecific type of intervention, as this blocks not only the IL-6 cytokine, but also numerous other cytokines and growth factors.

The anti-hIL-6 VHH antibodies described herein provide therapeutic inhibitors that target IL-6, and that may also target steps in the IL-6 signaling cascade. Furthermore, components of the IL-6 signaling cascade, such as JAKs, can be inhibited by small molecules.

### JAKS and STATS

Activation of the Janus tyrosine kinase (JAK) family members (JAK1, JAK2, and TYK2) leads to the activation of transcription factors of the signal transducer and activator of transcription (STAT) family. JAKS and STATS are critical components of many cytokine receptor systems. These components regulate growth, survival, differentiation and pathogen resistance. The IL-6 cytokine potently activates STAT3 and to a minor extent STAT1, For the IL-6 (or gp130) family of receptors (e.g., hIL-6R), which co-regulate B cell differentiation, plasmacytogenesis, and the acute phase reaction, cytokine (IL-6) binding induces receptor dimerization, activating the associated JAKS, which phosphorylate themselves and the IL-6 receptor. The phosphorylated sites on the receptor and JAKS serve as docking sites for the SH2-containing STATS, such as STAT3, and for SH2-containing proteins and adaptors that link the receptor to MAP kinase, PI3K/Akt, and other cellular pathways.

Phosphorylated STATS dimerize and translocate into the nucleus to regulate target gene transcription. Members of the suppressor of cytokine signaling (SOCS) family dampen receptor signaling via homologous or heterologous feedback regulation. JAKS or STATS can also participate in signaling through other receptor classes; STAT3 and STAT5 were found to be constitutively activated by tyrosine kinases other than JAKS in several solid tumors

The JAK/STAT pathway mediates the effects of cytokines, like erythropoietin, thrombopoietin, and G-CSF, which are protein drugs for the treatment of anemia, thrombocytopenia, and neutropenia, respectively. The pathway also mediates signaling by interferons, which are used as antiviral and antiproliferative agents. Dysregulated cytokine signaling can contribute to cancer. Aberrant IL-6 signaling contributes to the pathogenesis of autoimmune diseases, inflammation, and cancers such as prostate cancer and multiple myeloma. STAT3 can act as an oncogene and is constitutively active in many tumors. Crosstalk between cytokine signaling and EGFR family members is seen in some cancer cells.

Activating JAK mutations are major molecular events in human hematological malignancies. In addition, somatic acquired gain-of-function mutations of JAK1 were found in adult T cell acute lymphoblastic leukemia, Somatic activating mutations in JAK1, JAK2, and JAK3 have also been identified in pediatric acute lymphoblastic leukemia (ALL). Furthermore, JAK2 mutations have been detected around pseudokinase domain R683 (R683G or DIREED) in Down syndrome childhood B-ALL and pediatric B-ALL.

### Blocking IL-6

IL-6 has three distinct binding sites that interact with the IL-6 receptor for binding, The initial binding of IL-6 to its membrane-bound or soluble IL-6R is mediated via a first site (site I). Subsequently, the formation of an IL-6/IL-6R complex triggers the homodimerization of gp130, and IL-6 binds to one gp130 receptor via site II and to the second gp130 molecule via site III.

The IL-6 signaling cascade offers several alternatives for therapeutic intervention, including the anti-hIL-6 VHH antibody biologics described herein that can block the IL-6 cytokine from binding its receptor. Because IL-6 has been identified as the driving signal in a number of inflammatory diseases, the use of agents that block or inhibit IL-6 and IL-6 activity, such as the anti-hIL-6 VHH antibodies described herein, may ameliorates symptoms or even completely prevents the onset of disease. Consequently, blocking IL-6 through the use of anti-hIL-6 VHH antibodies as described herein is a rational therapy for patients and the inhibition of IL-6 and its activity is a valuable option for clinical use. Of note, the anti-hIL-6 VHH antibodies described herein that target IL-6 can be used in considerably lower amounts in patients compared with antibodies that block IL-6R, because sIL-6R is present in the serum at high concentrations. Consequently, all of the serum sIL-6R proteins need to be saturated with blocking antibodies before such anti-IL-6R antibodies have a pharmacodynamic effects and prevent IL-6 signaling.

In contrast, because IL-6 concentrations are low in healthy individuals, the anti-hIL-6 VHH anti bodies described herein that directly target the cytokine need only to bind (capture) newly synthesized and released IL-6 molecules in order to achieve a therapeutic effect. In addition, the direct blockade of IL-6 does not interfere with other cytokines that can signal through the IL-6R; therefore, direct binding of IL-6 by anti-hIL-6 VHH antibodies described herein offers the most direct mode of IL-6 inhibition IL-6 or IL-6R blockade is often successful in patients who are refractory to TNF inhibition and thus provides a viable treatment option for this group of patients. In addition, the anti-hIL-6 VHH antibodies described herein may be used not only to block or inhibit IL-6 and/or IL6 activity directly, but also to inhibit other pro-inflammatory mediators that are simultaneously blocked when JAKs are inhibited.

### Anti-hIL-6 VHH Antibodies

The anti-hIL-6 VHHs or multimeric forms thereof as described herein are provided as beneficial therapeutic agents that bind to human IL-6 protein (polypeptide). In some cases, the anti-hIL-6 VHHs or multimeric forms thereof bind to hIL-6 to block or inhibit the ability of hIL-6 to bind to its cellular receptor (IL-6R) and/or to promote the neutralization of adverse activity, function, or signaling by the hIL-6 cytokine. In some cases, an anti-hIL-6 VHH can also accelerate clearance of hIL-6 from the system to eliminate future adverse events or pathology. In addition, increased stability and longevity of the anti-hIL-6 VHHs or multimeric forms thereof in the circulation and in the body contribute to the advantages of these molecules as therapeutic agents that provide greater therapeutic efficacy as a treatment for hIL-6-related diseases and pathologies, and the symptoms thereof.

In some embodiments, the binding activity and/or neutralizing activity of the anti-hIL-6 VHHs described herein, or multimeric forms thereof, in the absence of any epitope tag sequences are significantly effective such that the hIL-6 binding and neutralization functions of these molecules obviates the need for an anti-tag antibody or clearing antibody.

VHHs, such as the anti-hIL-6 VHHs described herein, have a number of advantages over conventional antibodies and recombinant antibody domains, including (i) they are small monomeric proteins (14 kDa) that express and fold efficiently in recombinant hosts; (ii) they are more stable to extremes of pH and temperature compared with conventional antibodies; (iii) they typically bind conformational epitopes, and thus are more likely to neutralize target functions; and (iv) they are amenable to designed multimerization which often leads to higher potencies; and (v) they offer more therapeutic versatility, such as multispecificity, thus supporting their beneficial utility in treating diseases caused by or associated with hIL-6 and/or hIL-6 signaling.

The amino acid sequences of representative anti-hIL-6 VHH antibodies described herein are set forth in SEQ ID NOs: 1, 3, 5, 7 and 9, and the corresponding polynucleotide sequences encoding each of the representative anti-hTL-6 VHH antibodies are set forth in SEQ ID NOs: 2, 4, 6, 8 and 10 (Example 1). The binding regions of the anti-hIL-6 VHHs include CDRs (CDR1, CDR2 and CDR3) as set forth in the sequences of representative anti-hTL-6 VHHs described in Example 1 and presented in Table 3a below. The CDR binding regions are positioned within framework (FR) regions of the VHH polypeptide, which do not vary substantially in sequence between discrete anti-hTL-6 VHHs and which provide a “structural scaffold” for the CDRs, which bind to hTL-6. By way of non-limiting example, the binding of CDRs within FRs to a target protein (antigen), e.g., hIL-6, may be via conformational binding or interaction, electrostatic binding interaction, hydrogen bonding, Van der Waals forces, or hydrophobic bonding, or combinations thereof, as would be appreciated by those having skill in the art.

The CDRs of the anti-hTL-6 VHH polypeptides described herein may vary in amino acid sequence length. By way of nonlimiting example, CDR1 of the anti-hIL-6 VHH polypeptides as described herein may comprise from about 6 to about 12 (e.g., 8) amino acid residues; CDR2 may comprise from about 7 to about 12 (e.g., 8) amino acid residues; and CDR3 may comprise from about 8 to about 23 (e.g., 18-22) amino acid residues. It will be appreciated by one skilled in the art that number of amino acids that constitute a CDR is not necessarily precise. In some cases, an amino acid residue, or 2 or 3 amino acid residues, at one end or both ends of a given CDR may be considered as part of the CDR or as part of the neighboring FR region. The CDR regions of representative anti-hIL-6 VHH antibody polypeptides generated from camelid alpacas as described herein (Example 1) are presented in Table 3a below. In addition, FIG. 2 presents the amino acid sequences of a family of anti-hIL-6 VHH antibody polypeptides (JYK-D12 family) generated from a camelid alpaca immunized with hIL-6 polypeptide as described herein. Members of the JYK-D12 family of anti-hIL-6 VHH antibodies, which bind hIL-6, are also shown in Table 3a. The anti-hIL-6 VHH antibodies in FIG. 2 demonstrate the CDR diversity that is selected during affinity maturation of hIL-6 binding polypeptides in the same animal. Despite such CDR diversity, the hIL-6 binding VHHs generated as described herein show detectable binding to hIL-6. As observed from the sequence alignments shown in FIG. 2, in the context of the four VHH framework regions, which do not vary significantly in sequence among different anti-hIL-6 VHH polypeptides, the anti-hIL-6 VHH polypeptides demonstrate significant binding to the hIL-6 antigen, despite some variation among the CDR sequences in the context of their framework regions.

TABLE 3a

| VHH | | | | | | |
|---|---|---|---|---|---|---|
| (anti- | | SEQ | | SEQ | | SEQ |
| IL-6 | | ID | | ID | | ID |
| VHH) | CDR1 | NO: | CDR2 | NO: | CDR3 | NO: |
| JYK- | GFTLDYYA | 11 | SSSDLKTY | 16 | GTWDLKFGYNISACVGSYEYDY | 17 |
| A1 | | | | | | |
| JYK- | GRPFSSFA | 31 | TWSRGTTH | 32 | AAADGWKVVSTASPAYDY | 33 |
| A9 | | | | | | |
| JYK- | GFTLAYYG | 24 | SSSDLSTY | 25 | GTWDLKFGYSRSNCVRSYEYDY | 26 |
| D12 | | | | | | |
| JYK- | GRTFSSRA | 34 | SWTGSPY | 35 | AATSEHVMLVVTTRGGYDY | 36 |
| F12 | | | | | | |
| JYK- | GFTLDYYA | 11 | SSSDRSTY | 12 | GTWDLKWGYNISACVGSYEYDY | 13 |
| H9 | | | | | | |

Table 3b shows the FR1-FR4 regions of the anti-IL-6 VHH proteins of Table 3a:

TABLE 3b

| VHH | | | | |
|---|---|---|---|---|
| (anti-IL- | | SEQ ID | | SEQ ID |
| 6 VHH) | FR1 | NO: | FR2 | NO: |
| JYK-A1 | QLQLAETGGGLVQP | 76 | VGWFRQAPGKEREGISCI | 62 |
| | GGSLRLSCAAS | | | |
| JYK-A9 | QVQLVESGGGLVQA | 77 | MGWFRQAPGKEREFVAAI | 81 |
| | GDSLTLSCAAS | | | |
| JYK-D12 | QLQLVESGGGLVQP | 78 | IGWFRQAPGKEREGVACI | 65 |
| | GGSLGLSCAAS | | | |
| JYK-F12 | QVQLAETGGGSVQA | 79 | MGWFRQAPGKEREFVAVI | 82 |
| | GGSLTLSCAAS | | | |
| JYK-H9 | QLQLVETGGGLVQP | 80 | IGWFRQAPGKEREGVSCL | 61 |
| | GGSLRLSCAAS | | | |
| VHH | | | | |
| (anti-IL- | | SEQ ID | | SEQ ID |
| 6 VHH) | FR3 | NO: | FR4 | NO: |
| JYK-A1 | YADSVKGRFTISRDYAK | 69 | WDQGTQVTVSS | 74 |
| | STVSLQMNSLKPEDTG | | | |
| | VYYCAA | | | |
| JYK-A9 | YADSVKGRFTISGDNA | 83 | WGQGTQVTVSS | 50 |
| | KNTVFLQMNSLKPEDT | | | |
| | AVYYC | | | |
| JYK-D12 | YADSVKGRFTISRDNA | 73 | WGQGTQVTVSS | 50 |
| | KDTVYLQMNSLKPEDT | | | |
| | AVYYCAA | | | |
| JYK-F12 | YTDSVKGRFTISRDDA | 84 | WGQGTQVTVSS | 50 |
| | KNTVYLQMNSLKPEDT | | | |
| | AVYYC | | | |
| JYK-H9 | YVDSVKGRFTISRDDD | 66 | WGQGTQVTVSS | 50 |
| | KNTAYLQMNSLKPEDT | | | |
| | ATYYCAA | | | |

In view of the representative anti-hIL-6 VHH amino acid sequences shown in FIG. 2, it will be appreciated by one skilled in the art that individual VHH polypeptides, (e.g., of about 125 amino acids in length and comprising 3 CDRs and 4 FR regions), which comprise at least about or equal to 85%, or 88%, or greater identity in amino acid sequence bind to hIL-6 antigen. In addition, the hIL-6 binding VHHs may further neutralize hIL-6 activity. In an embodiment, at least about or equal to 85%, 86%, 87%, 88%, 89%, 90%, 91%, 92%, 93%, 94%, 95%, 96%, 97%, 98%, 99%, or 100% amino acid sequence identity is tolerated among the anti-hIL-6 VHHs without adversely affecting or eliminating binding of the VHH polypeptides to the hIL-6 antigen. In an embodiment, such amino acid sequence variation among the anti-hIL-6 VHH polypeptides is tolerated in the CDRs of the VHH polypeptides without adversely affecting binding of the VHHs to hIL-6. In a particular embodiment, the amino acid sequence variations between or among anti-hIL-6 VHHs encompass one or more conservative amino acid substitutions or changes in a VHH amino acid sequence. In an embodiment, the one or more conservative amino acid substitutions or changes in a VHH amino acid sequence occur in one or more CDR sequences of the VHH, in one or more FR sequences of the VHH, or in CDR and FR sequences of the VHH.

The three CDRs of the anti-hIL-6 VHH polypeptides are arranged or positioned in the context of four FR regions as follows: FR1-CDR1-FR2-CDR2-FR3-CDR3-FR4, in which FR1 to FR4 refer to the framework regions 1-4, respectively, and in which CDR1 to CDR3 refer to the complementarity determining regions 1-3, respectively. An alignment of anti-hIL-6 VHHs, all of which specifically bind to hIL-6 protein antigen, demonstrates the extensive similarities among the sequences of each of the FRs (FR1, FR2, FR3 and FR4) found in the different hIL-6-binding VHH polypeptides (FIG. 2). Similar to the FRs in conventional antibody polypeptides, the respective FRs (FR1, FR2, FR3 and FR4) of the anti-hIL-6 VHH polypeptides described herein are highly similar in sequence among different hIL-6-binding VHHs that were generated. Accordingly, provided are anti-hIL-6 VHH polypeptides comprising CDR1-3, in the structural context of FR1-4, that bind to and/or neutralize hIL-6 protein, or to suitable fragments of the hIL-6 protein, as well as polypeptides that comprise or consist essentially of one or more of the anti-hIL-6 VHHs and/or hIL-6 binding fragments thereof.

In addition, the FRs of the hIL-6-binding VHHs described herein are highly or essentially similar in sequence to the FRs of VHHs produced in camelid animals, such as alpacas, camels, llamas, and the like. As they provide structural and conformational support for the CDRs of VHH polypeptides, the FRs and the FR1, FR2, FR3 and FR4 regions among camelid VHH polypeptides generally share significant sequence identity. (FIG. 9). See, e.g., A. M. Vattekatte et al., March, 2020, PeerJ., 6(8):e8408. DOI: 10.7717/peerj.8408 and L. S. Mitchell and L. J. Colwell, 2018, Proteins, 86(7): 697-706).

Table 2 presents the amino acid sequences of the four framework regions, i.e., FR1, FR2, FR3 and FR4, respectively, of 15 representative anti-hIL-6 VHH polypeptides described herein (i.e., XAX-C9, XAX-H12, XAX-H9, JYK-H9, JYK-A8, JYK-G1, XAX-E6, JYK-A1, JYK-F6, XAX-G8, JYK-G10. XAX-B7, XAX-H5, XAX-C2, JYK-D12); Table 3b presents the amino acid sequences of the four framework regions, i.e., FR1, FR2, FR3 and FR4, respectively, of anti-hIL-6 VHH polypeptides JYK-A1, JYK-A9, JYK-D12, JYK-F12, and JYK-H9, which are JYK-D12 VHH family members, and FIG. 2 presents the amino acid sequences of several identified anti-hTL-6 VHH polypeptides relative to each other, with the FR and CDR regions shown. The alignment of the sequences supports substantial similarity among the structural FRs of the anti-hIL-6 camelid VHH antibodies described herein.

In embodiments, in cases in which a FR (or CDR) amino acid residue in a VHH polypeptide may be one of several alternative amino acid residues, the alternative amino acid residues will frequently share similar characteristics or properties, e.g., hydrophobicity, polarity, and/or charge. A conservative replacement (also called a conservative substitution) is an amino acid replacement or substitution in a polypeptide or region thereof that changes a given amino acid residue to a different amino acid residue with similar biochemical properties, such as charge, hydrophobicity, and/or size. By way of non-limiting example, the below Table 4 presents amino acids and their 1-letter codes categorized into six main classes based on their structure and the general chemical characteristics of their side chains (R groups).

TABLE 4

| Amino Acids | Class |
|---|---|
| Glycine (G), Alanine (A), Valine (V), Leucine | Aliphatic |
| (L), Isoleucine (I) | |
| Serine (S), Cysteine (C), Selenocysteine (U), | Hydroxyl or sulfur/ |
| Threonine (T), Methionine (M) | selenium containing |
| Proline (P) | Cyclic |
| Phenylalanine (F), Tyrosine (Y), Tryptophan | Aromatic |
| (W) | |
| Histidine (H), Lysine (K), Arginine (R) | Basic |
| Aspartate (D), Glutamate (E), Asparagine (N), | Acidic and amides |
| Glutamine (Q) | thereof |

In an embodiment, amino acid sequence substitutions or changes in an anti-hIL6 VHH polypeptide relative to another anti-h-IL6 VHH polypeptide comprise conservative amino acid substitutions or changes such that a given amino acid residue is substituted with or replaced by a different amino acid residue with similar biochemical properties, such as charge, hydrophobicity, and/or size. In an embodiment, sequence variation between or among anti-hIL6 VHH polypeptides results from one or more conservative amino acid changes and account for the percent sequence variation, e.g., 85%, 86%, 87%, 88%, 90%, 91%, 92%, 93%, 94%, 95%, 96%, 97%, 98%, or 99% sequence variation.

In some embodiments, the VHHs as described herein are humanized using methods and techniques practiced by those having skill in the art. (See, e.g., U.S. Pat. Nos. 8,975,382 and 10,550,174, the contents of which are incorporated by reference herein).

The anti-hIL6 VHH antibodies described herein have widespread application as therapeutics in the treatment of diseases, disorders, conditions, pathologies, and infection associated with the presence and/or activity of the IL-6 cytokine. The described anti-hIL6 VHHs described herein are particularly useful for binding to and neutralizing the IL-6 cytokine, and, in some cases, to blunt, reduce, ameliorate, or eliminate the debilitating effects of cytokine storm associated with cellular production and release of IL-6 in a subject. In embodiments, the invention encompasses polynucleotides (nucleic acid sequences) that encode the operably linked modular components that constitute the described anti-hIL-6 VHHs. In embodiments, the anti-hIL-6 VHHs are recombinantly produced. In embodiments, the anti-hIL-6 VHHs encompass the proteins (polypeptides) encoded by the polynucleotides. In embodiments, the polynucleotide is DNA, cDNA, RNA, mRNA, and the like. In an embodiment, the anti-hIL-6 VHHs may be humanized or codon-optimized using methods practiced by those having skill in the art.

Polynucleotides Encoding VHHs that Bind to Human Interleukin 6 (hIL-6)

In some cases, more than one anti-hIL-6-binding VHH antibody (i.e., anti-hIL-6 VHH) is coupled or linked (e.g., covalently linked) to other sequences, e.g., a leader amino acid sequence, one or more spacer or linker (flexible spacer or linker) amino acid sequences, or one or more epitope tag amino acid sequences, to produce a multimeric VHH binding molecule containing two or more, e.g., three, four, five, or six, VHHs linked together. In an embodiment, a polynucleotide molecule, such as a recombinant or isolated polynucleotide molecule, encodes a single anti-hIL-6 VHH or more than one anti-hIL-6 VHH linked together to form a multimer (i.e., a multimeric anti-hIL-6 VHH binding molecule). In an embodiment, the polynucleotide encodes a fragment or portion of the anti-hIL-6 VHH or multimeric anti-hIL-6 VHH binding molecule, in particular, a fragment or portion that maintains hIL-6 binding activity or hIL-6 binding and neutralizing activities. The polynucleotide sequences encoding representative anti-hIL-6 VHH antibodies as described herein are set forth in SEQ ID NOs: 2, 4, 6, 8 and 10 (Example 1).

In an embodiment, an anti-hIL-6 VHH can be humanized, i.e., modified to increase its similarity to antibodies or antibody variants produced naturally in humans, using techniques known and practiced in the art. Briefly and by way of nonlimiting example, a humanized antibody can be generated by inserting the appropriate CDR coding sequences (e.g., ‘donor’ sequences that are responsible for the desired binding properties) into a human antibody “scaffold” (e.g., ‘acceptor’ sequences) comprising essentially invariant framework region (FR) sequences (FRs). In embodiments, the CDRs of the anti-hIL-6 VHH antibodies described herein may be inserted into FRs, which provide the structural scaffold that allows the CDRs to bind to, and in certain cases, to neutralize, hIL-6. Recombinant DNA methods using an appropriate vector and expression in mammalian cells are employed and routinely practiced in the art to achieve the production of recombinant humanized antibodies.

In an embodiment, the polynucleotide encodes a hIL-6-binding VHH molecule having binding and neutralizing function, or a functional binding portion thereof, that includes an epitope tag. In embodiments, antibody fragments, microproteins, darpins, anticalins, peptide mimetic molecules, aptamers, synthetic molecules, etc. can be linked to the multimeric anti-hIL-6 VHH binding molecule. In embodiments, a multimeric anti-hIL-6 VHH binding molecule may contain two of the same anti-hIL-6 VHHs, e.g., a dimeric form, or two different anti-hIL-6 VHHs described herein. In other embodiments, a multimeric anti-hIL-6 VHH binding molecule may contain more than two anti-hIL-6 VHHs in combination, e.g., a combination of three, four, or five, etc. anti-hIL-6 VHHs linked together. In an embodiment, the anti-hIL-6 VHH components of a multimeric anti-hIL-6 VHH binding molecule may be linked covalently.

In an embodiment, an anti-hIL-6 VHH can be modified, for example, by attachment (e.g., either directly or indirectly via a linker or spacer) to another anti-hIL-6 VHH. In some embodiments, an anti-hIL-6 VHH is attached or genetically (recombinantly) fused to another anti-hIL-6 VHH. Accordingly, a polynucleotide (e.g., DNA) that encodes one anti hIL-6 VHH is joined (in reading frame) with the polynucleotide encoding a second anti-hIL-6 VHH, and so on. In certain embodiments, additional amino acids are encoded within the polynucleotide between the anti-hIL-6 VHHs so as to produce an unstructured region (e.g., a flexible spacer) that separates the anti-hIL-6 VHHs, e.g., to better promote independent folding of each anti-hIL-6 VHH antibody into its active or functional conformation or shape. Commercially available techniques for fusing proteins (or their encoding polynucleotides) may be employed to recombinantly join or couple the anti-hIL-6 VHHs into multimeric anti-hIL-6 VHHs containing two or more of the same or different anti-hIL-6 VHHs as described herein.

Polynucleotide sequences encoding the anti-hIL-6 VHHs or multimeric forms thereof as described herein can be recombinantly expressed and the resulting encoded anti-hIL-6 VHH antibody molecules can be produced at high levels and isolated and/or purified. In an embodiment, the recombinant anti-hIL-6 VHHs or multimeric forms thereof are produced in soluble form. In an embodiment, a recombinantly produced anti-hIL-6 VHH is dimeric, such that two anti-hIL-6 VHHs, same or different, are joined or linked together, either directly or indirectly. In an embodiment, a recombinantly produced anti-hIL-6 VHH is multimeric, e.g., a tetramer, which contains four anti-hIL-6 VHH antibodies, the same or a combination of different anti-hIL-6 VHHs, joined together. By way of example, a tetramer may contain four of the same anti-hIL-6 VHHs joined together, or a combination of four different anti-hIL-6 VHHs, or two pairs of the same anti-hIL-6 VHHs, joined together. In an embodiment, the anti-hIL-6 VHH or multimeric forms thereof are contained in pharmaceutically acceptable compositions for use in treating a disease, disorder, pathology, or infection associated with or caused by excess amounts, levels, or production of IL-6, or with dysregulation of IL-6 and/or IL-6 signaling, such as infections (e.g., viral or bacterial infections); oncological diseases (cancers, carcinomas, tumors, and the like), e.g., cholangiocarcinoma, ovarian cancer, and multiple myeloma; immune-mediated diseases (autoimmune diseases and inflammatory diseases), e.g., adult rheumatoid arthritis, juvenile idiopathic arthritis, Castleman's disease, secondary amyloidosis, polymyalgia rheumatic, adult onset Still's disease, polymyositis, systemic sclerosis, large vessel vasculitis lupus erythematosus, Crohn's disease, irritable bowel disease (IBD), Sjogren's syndrome; steroid refractory Graft versus Host Disease in transplantation; type 2 diabetes, obesity and schizophrenia.

The compositions and methods described herein in various embodiments include an isolated polynucleotide sequence or an isolated polynucleotide molecule that encodes an anti-hIL-6 VHH or multimeric form thereof. Accordingly, in some embodiments, the isolated polynucleotide sequence or isolated polynucleotide molecule comprises or consists of a polynucleotide sequence that encodes a polypeptide molecule (anti-hIL-6 VHH) having an amino acid sequence of SEQ ID NOS: 1, 3, 5, 7, 9, or a functional portion thereof, as described herein. In some embodiments, the isolated polynucleotide sequence or isolated polynucleotide molecule comprises or consists of a polynucleotide sequence of SEQ ID NOS: 2, 4, 6, 8, or 10. In an embodiment, a composition comprises a combination of the isolated polynucleotide sequences or isolated polynucleotide molecules as described herein.

Also encompassed by the present invention are polynucleotide sequences, DNA or RNA, which are substantially complementary to the DNA sequences encoding the polypeptides described herein, and which specifically hybridize with these DNA sequences under conditions of stringency known to those of skill in the art. As referred to herein, substantially complementary means that the nucleotide sequence of the polynucleotide need not reflect the exact sequence of the original encoding sequences, but must be sufficiently similar in sequence to permit hybridization with a nucleic acid sequence under high stringency conditions. For example, non-complementary bases can be interspersed in a nucleotide sequence, or the sequences can be longer or shorter than the polynucleotide sequence, provided that the sequence has a sufficient number of bases complementary to the sequence to allow hybridization thereto. Conditions for stringency are described, e.g., in Ausubel, F. M., et al., Current Protocols in Molecular Biology, (Current Protocol, 1994), and Brown, et al., Nature, 366:575 (1993); and further defined in conjunction with certain assays.

Vectors and plasmids containing one or more of the polynucleotide molecules encoding the anti-hIL-6 VHH amino acid sequence of SEQ ID NO: 1, 3, 5, 7, 9, or a functional portion thereof, are provided. Vectors and plasmids containing one or more of the polynucleotide molecules of SEQ ID NOS: 2, 4, 6, 8, or 10 are also provided. Suitable vectors for use in eukaryotic and prokaryotic cells are known in the art and are commercially available or readily prepared by the skilled practitioner in the art. Additional vectors can also be found, for example, in Ausubel, F. M., et al., Ibid. and in Sambrook et al., “Molecular Cloning: A Laboratory Manual,” 2nd ED. (1989), and other editions.

Any of a variety of expression vectors (prokaryotic or eukaryotic) known to and used by those of ordinary skill in the art may be employed to express recombinant polypeptides described herein. Expression can be achieved in any appropriate host cell that has been transformed or transfected with an expression vector containing a polynucleotide (DNA) molecule that encodes a recombinant polypeptide. Suitable host cells include prokaryotes, yeast and higher eukaryotic cells. By way of example, the host cells employed include, without limitation, E. coli, yeast, insect cells, or a mammalian cell line such as COS or CHO. The DNA sequences expressed in this manner can encode any of the polypeptides described herein, including variants thereof.

Uses of plasmids, vectors or viruses (viral vectors) containing polynucleotides encoding the anti-hIL-6 VHHs or multimeric forms thereof as described herein include generation of mRNA or protein in vitro or in vivo. In related embodiments, host cells transformed with the plasmids, vectors, or virus vectors are provided, as described above. Nucleic acid molecules can be inserted into a construct (such as a prokaryotic expression plasmid, a eukaryotic expression vector, or a viral vector construct, which can, optionally, replicate and/or integrate into a recombinant host cell by known methods. The host cell can be a eukaryote or prokaryote and can include, for example and without limitation, yeast (such as Pichia pastoris or Saccharomyces cerevisiae), bacteria (such as E. coli, or Bacillus subtilis), animal cells or tissue (CHO or COS cells), insect Sf9 cells (such as baculoviruses infected SF9 cells), or mammalian cells (somatic or embryonic cells, Human Embryonic Kidney (HEK) cells, Chinese hamster ovary (CHO) cells, HeLa cells, human 293 cells (Expi293F), and monkey COS-7 cells). Suitable host cells also include a mammalian cell, a bacterial cell, a yeast cell, an insect cell, a plant cell, or an algal cell.

An anti-hIL-6 VHH-encoding polynucleotide molecule can be incorporated or inserted into the host cell by known methods. Examples of suitable methods for transfecting or transforming host cells include, without limitation, calcium phosphate precipitation, electroporation, microinjection, infection, lipofection and direct uptake. “Transformation” or “transfection” as appreciated by the skilled practitioner refers to the acquisition of new or altered genetic features by the incorporation of additional nucleic acids, e.g., DNA, into a cell and/or into cellular DNA. “Expression” of the genetic information of a host cell is a term of art which refers to the directed transcription of DNA to generate RNA that is, in turn, translated into a polypeptide (anti-hIL-6 VHH antibody). Procedures for preparing recombinant host cells and incorporating nucleic acids are described in more detail in Sambrook et al., “Molecular Cloning: A Laboratory Manual,” Second Edition (1989) and Ausubel, et al. “Current Protocols in Molecular Biology,” (1992), and later editions, for example.

A transfected or transformed host cell is maintained under suitable conditions for expression and recovery of the polypeptides described herein. In certain embodiments, the cells are maintained in a suitable buffer and/or growth medium or nutrient source for growth of the cells and expression (and secretion) of the gene product(s) into the growth medium. The type of growth medium is not critical to the invention and is generally known to those skilled in the art, such as, for example, growth medium and nutrient sources that include sources of carbon, nitrogen and sulfur. Examples include Luria-Bertani (LB) broth, Superbroth, Dulbecco's Modified Eagles Media (DMEM), RPMI-1640, M199 and Grace's insect media. The growth medium can contain a buffering agent, as commonly used in the art. The pH of the buffered growth medium may be selected and is generally a pH that is tolerated by, or optimal for, growth of the host cell, which is maintained under a suitable temperature and atmosphere.

In another aspect, an RNA polynucleotide, in particular, mRNA, encodes the anti-hIL-6 VHHs or multimeric forms thereof as described herein. mRNA encoding the anti-hIL-6 VHHs or multimeric forms thereof may contain a 5′ cap structure, a 5′ UTR, an open reading frame, a 3′ UTR and poly-A sequence followed by a C30 stretch and a histone stem loop sequence (Thess, A. et al., 2015, Mol Ther, 23(9):1456-1464; Thran, M. et al., 2017, EMBO Molecular Medicine, DOI: 10.15252/emmm.201707678). Sequences may be codon-optimized for human use using techniques and protocols known and used by those skilled in the art. In an embodiment, the mRNA sequences do not include chemically modified bases. mRNAs encoding the anti-hIL-6 VHHs or multimeric forms thereof as described herein may be capped enzymatically or further polyadenylated for in vivo studies/use. In an embodiment, an anti-hIL-6 VHH monomer or multimer, e.g., a homodimer, is encoded by a mRNA molecule. In an embodiment, the mRNA encoding the anti-hIL-6 VHH monomer or homodimer may be delivered to or introduced into a cell.

Expression of proteins, which normally have a shortened serum half-life, by encoding mRNA, particularly sequence optimized, unmodified mRNA, advantageously prolongs the bioavailability of these proteins for in vivo activity. (see, e.g., K. Kariko et al, 2012, Mol. Ther., 20:948-953; Thess, A. et al., 2015, Mol Ther, 23(9):1456-1464;). Accordingly, anti-hTL-6 VHHs or multimeric forms thereof with an estimated serum half-life of 1-2 days are likely to benefit from being encoded by mRNA. Of note, the half-lives of neutralizing VHH protein serum titers at one to three days after treatment were estimated to be, on average, 1.5-fold higher than from day three onward, even without target-specific mRNA optimization. (Mukherjee et al., 2014, PLoS ONE, 9e106422). In general, one to three days after treatment, both mRNA and protein half-lives contribute to the kinetics of serum titers, while after day three forward, the kinetics is almost exclusively determined by the properties of the expressed protein.

### Multimeric Forms of the Anti-hIL-6 VHHs

Multimeric forms of the anti-hIL-6 VHH antibodies described herein are encompassed by the present disclosure. Such multimeric anti-hIL-6 VHHs contain more than one anti-hIL-6 VHH antibody that binds to hIL-6. In an embodiment, a multimer of anti-hIL-6 VHH antibodies contains two anti-hIL-6 VHHs, same or different, that bind to hIL-6. Such a multimeric form of the anti-hIL-6 VHH molecules constitutes a dimeric multimer. In an embodiment, the dimeric multimer comprises two of the same anti-hIL-6 VHH antibodies coupled using a flexible linker. In an embodiment, the dimeric multimer comprises two, different anti-hIL-6 VHH antibodies coupled using a flexible linker. In an embodiment, the two, different anti-hIL-6 VHH antibodies bind to different, nonoverlapping epitopes of hIL-6.

In another embodiment, a multimer of anti-hIL-6 VHH antibodies contains more than two (e.g., three, four, five, six, etc.) anti-hIL-6 VHHs, same or different, that bind to hIL-6. Such a multimeric form of the anti-hIL-6 VHH molecules may comprise three or more of the same anti-hTL-6 VHH antibodies coupled together directly or indirectly using flexible linkers. In an embodiment, the anti-hIL-6 VHH multimer comprises a combination or mixture of the anti-hIL-6 VHH antibodies described herein coupled using a flexible linker. In some cases, the multimeric form of the anti-hIL-6 VHH antibodies may contain more than one of the same anti-hIL-6 VHH antibody and/or different, or different combinations of, anti-hIL-6 VHH antibodies coupled using flexible linker or spacer peptides. Nonlimiting examples of flexible linking amino acid sequences include amino acid sequence (GGGGS)ₙ (SEQ ID NO: 85), where, without limitation, n may be 1-30, or 1-20, or 1-10, or 1-5, e.g., GGGGS (SEQ ID NO: 43); GGGGSGGGGSGGGGS ((GGGGS)₃) (SEQ ID NO: 44), or a functional portion thereof; EPKTPKPQGGGGSGGGGSGGGGSQGVQSQVQLVE (SEQ ID NO: 45); or EPKTPKPQ (SEQ ID NO: 46). In certain embodiments, the anti-hTL-6 VHH amino acid sequences described herein are coupled to epitope tag amino acid sequences as described infra, or to other sequences. In another embodiment, a dimerization agent that complexes peptide fragments each containing at least about 5 to 25 amino acids, 25 to 50 amino acids, 50 to 100 amino acids, 100 to 150 amino acids, and 150 amino acids to about 200 amino acids may be used. Multimerization agents and methods of using the agents for forming multimeric binding proteins can be found, for example, in U.S. Pat. Nos. 9,023,352, 8,349,326 and 7,763,445, each of which is incorporated by reference herein in its entirety.

In embodiments, the multimeric forms of the anti-hTL-6 VHH antibodies described herein both bind to hIL-6 and neutralize its activity.

### Epitope and Fc Tags and Antibodies Thereto

In certain embodiments, an anti-hIL-6 VHH antibody, or a dimeric or multimeric form thereof, includes a single epitope tag (single tag sequence) or multiple tags (multiple tag sequences), to which anti-tag antibodies specifically bind. For example, a multimeric VHH may include at least one, or two or more, epitope tags in the molecule. Such epitope tags, which are specifically bindable by the anti-epitope tag antibodies, are useful in detecting VHHs bound to hIL-6 protein antigen. In addition, in some cases, such tags may facilitate clearance of VHHs bound to antigen following binding of the tags by anti-tag antibody. By way of nonlimiting example, a tag may constitute an O-tag epitope of amino acid sequence DELGPRLMGK (SEQ ID NO: 41) or an E-tag epitope of amino acid sequence GAPVPYPDPLEPR (SEQ ID NO: 42). The epitope tags may be placed at the amino terminus, carboxy terminus, or internally within a multimeric VHH molecule. Such tags and/or anti-tag antibodies are described for example, in (U.S. Pat. Nos. 8,349,326; 9,023,352, WO 2019/094095A1) and U.S. Pat. Nos. 7,943,345; 8,114,634 and 8,865,871), the contents of which are incorporated herein by reference in their entireties. An example of an anti-O tag monoclonal antibody (IgG1) suitable for binding the DELGPRLMGK (SEQ ID NO: 41) tag sequence is described in WO 2019/094095A1, the contents of which are fully incorporated by reference. By way of illustrative example, peroxidase labeled antibodies that bind the anti-O-tag antibody may be used to detect these anti-tag antibodies in assays in which samples are incubated with goat anti-O-tag-HRP conjugated antibody (Bethyl labs) diluted 1:5000 in blocking solution for 1 hour at RT with rocking and were washed as above before adding TMB microwell peroxidase substrate (KPL) to develop (incubated for 10-40 min). Development was stopped with 1M H₂SO₄ and the plates were read at 450 nm on an ELx808 Ultra Microplate Reader (Bio-Tek instruments), (Mukherjee, J. et al., 2012, PloS ONE 7:e29941).

In some cases, an albumin binding peptide (DICLPRWGCLWED (SEQ ID NO: 86)), may be included at the 3′ end of an anti-hTL-6 VHH antibody or multimeric form thereof.

In certain embodiments, the presence of an epitope tag operably linked, coupled, or fused to a VHH antibody or multimeric form thereof, wherein the tag is bound by an anti-epitope tag antibody, induces clearance of the hIL-6-bound VHH molecule from the body. In an embodiment, the binding of one or more epitope tags in an anti-hTL-6 VHH molecule by anti-epitope tag antibody(ies) may synergistically induce clearance of hIL-6 from the body following binding by the VHH or multimeric form thereof.

In an aspect, an anti-tag (i.e., anti-epitope tag) antibody may be administered to a subject who is also treated with or administered an anti-hIL-6 VHH or multimeric form thereof containing one or more epitope tags, or a pharmaceutical composition thereof. The anti-tag antibodies bind to the epitope tags of the anti-hIL-6 VHH, which, in turn, binds to one or more hIL-6 proteins, thereby forming a complex that is rapidly cleared from the body (Sepulveda, J. et al., 2010, Infect. Immun., 78(2):756-763; Mukherjee, J. et al., 2012, PLoS ONE, 7(1). e29941 PMCID: PMC3253120; https://doi.org/10.1371/journal.pone.0029941). In an embodiment, an anti-epitope tag monoclonal antibody of a specific isotype, for example IgG1, or a binding fragment or portion thereof that binds to the tag sequence, or a molecule containing its CDR components that bind to the tag sequence, may be provided to a subject who is also administered one or more anti-hIL-6 VHHs or a multimeric form thereof as described herein. The administration or co-administration of an anti-tag antibody advantageously enhances clearance from the body of a complex formed by hIL-6 bound by anti-hIL-6 VHH or a multimeric form thereof, which is, in turn, bound by an anti-epitope tag antibody or binding portion thereof.

In certain embodiments, an anti-tag antibody may also affect or facilitate immunoglobulin effector functions. Anti-tag antibodies may include, for example, IgA, IgD, IgE, IgG, and IgM immunoglobulins and subtypes thereof. An immune response to an epitope tag included in an anti-uIL-6 VHH or multimeric form thereof may involve the elicitation of specific monoclonal antibodies and/or polyclonal antibodies that specifically bind to the tag. Immunoglobulin effector functions may involve, for example, interaction(s) between the Fc portion of the immunoglobulin and receptors or other protein molecules in a subject or cells thereof. Depending on the immunoglobulin type, the effector functions result in clearance of the disease agent (e.g., excretion, degradation, lysis or phagocytosis). In an embodiment, an anti-tag antibody of one immunoglobulin effector type binds to an anti-hTL-6 VHH or multimeric form thereof which comprises one or more epitope tags. In embodiments in which a multimeric form of an anti-hIL-6 VHH comprises at least one epitope tag, or two or more epitope tags, an anti-tag antibody, or binding portion thereof, binds to each of the tags of the multimeric molecule. In embodiments, the epitope tags may be the same or different in a given anti-hIL-6 VHH multimeric molecule. Without wishing to be bound by theory, the presence of more than one epitope tag bindable by an anti-epitope tag antibody, or binding portion thereof, in a multimeric form of an anti-hIL-6 VHH may increase the rate and/or level of clearance of hIL-6 bound to the anti-hIL-6 VHH multimer in a subject.

In some embodiments, an immunoglobulin Fc region or portion thereof, e.g., having effector or modulator function (Fc tag), is coupled, fused, or linked to an anti-hIL-6 VHH antibody, or a dimer or multimer thereof, as described herein. In particular, Fc tags comprise a domain (effector domain) of an immunoglobulin molecule, e.g., IgG, which can be genetically (recombinantly) linked to a peptide or protein. Fc fusion proteins (also known as Fc chimeric fusion proteins, Fc-Igs, Ig-based chimeric fusion proteins, and Fc-tag proteins) are composed of an Ig Fc domain that is fused, linked, or coupled (e.g., by recombinant techniques) to a peptide or protein, such as an anti-hIL-6 VHH antibody described herein. The Fc domain portion of the fusion protein confers an advantageous characteristic to the anti-hIL-6 VHH antibody protein, particularly in vivo, by greatly prolonging the half-life of the protein in plasma following administration to a subject. In an embodiment, an anti-hIL-6 VHH antibody fused to an Fc region or Fc tag provides improved therapeutic efficacy as a biotherapeutic agent or drug. In an embodiment, an antibody directed to an Fc portion of the Fc-tagged anti-hIL-6 VHH antibody may be used. In an embodiment, the anti-Fc antibody is labeled or coupled to a detectable moiety or agent or reporter molecule.

Suitable methods of producing or isolating antibody fragments having the requisite binding specificity and affinity for binding to an epitope tag include for example, methods which select recombinant antibody from a library or by PCR (e.g., U.S. Pat. Nos. 5,455,030 and 7,745,587 each of which is incorporated by reference herein in its entirety).

Functional fragments of antibodies, including fragments of chimeric, humanized, primatized, veneered, or single chain antibodies, can also be produced. Functional fragments or portions of the foregoing antibodies include those which are reactive with the hIL-6 protein. For example, antibody fragments capable of binding to hIL-6 or a portion thereof, include, but not limited to, scFvs, Fabs, VHHs, Fv, Fab, Fab′ and F(ab′)₂. Such fragments can be produced by enzymatic cleavage or by recombinant techniques. For instance, papain or pepsin cleavage are used generate Fab or F(ab′)₂ antibody fragments, respectively. Antibody fragments are produced in a variety of truncated forms using antibody-encoding genes in which one or more stop codons has been introduced upstream of the natural stop site. For example, a chimeric gene encoding a F(ab′)₂ heavy chain peptide portion can be designed to include DNA sequences encoding the CH₁ peptide domain and hinge region of an immunoglobulin heavy chain.

### Pharmaceutical Compositions

Also featured herein are methods for treating or preventing pathologies, disorders, and diseases caused by or associated with hIL-6, hIL-6 cytokine storm, or dysregulation of hIL-6 signaling. The methods include administering to a subject in need thereof an amount of an anti-hIL-6 VHH or multimeric anti-hIL-6 VHH binding molecule that is effective to specifically bind to and optimally neutralize hIL-6 activity. In an embodiment, if an anti-hIL-6 VHH or multimeric anti-hIL-6 VHH binding molecule includes an epitope tag, an anti-epitope tag antibody may be administered to the subject. (see, e.g., WO 2019/094095A1, the contents of which is incorporated by reference herein in its entirety). In an embodiment, an anti-hIL-6 VHH or multimeric anti-hIL-6 VHH binding molecule is provided or used in a pharmaceutical composition.

Typically, a carrier or excipient is included in a composition as described herein, such as a pharmaceutically acceptable carrier or excipient, which includes, for example, sterile water, aqueous saline solution, aqueous buffered saline solutions, aqueous sucrose, dextrose, or mannose solutions, aqueous glycerol solutions, ethanol, calcium carbonate, albumin, starch, cellulose, silica gel, polyethylene glycol (PEG), dried skim milk, rice flour, magnesium stearate, and the like, or combinations thereof. The terms “pharmaceutically acceptable carrier” and a “carrier” refer to any generally acceptable excipient or drug delivery device that is relatively inert and non-toxic.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's The Science and Practice of Pharmacy Ed. by LWW 21^{st} EQ. PA, 2005 discloses various carriers used in formulating pharmaceutical compositions and known techniques for the preparation thereof. Carriers are selected to prolong dwell time for example following any route of administration, including IP, IV, subcutaneous, mucosal, sublingual, inhalation or other form of intranasal administration, or other route of administration.

Some examples of materials that can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The preparation of such compositions and solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, intravenous, oral, and the like.

In certain embodiments, these compositions optionally further comprise one or more additional therapeutic agents. In certain embodiments, the additional therapeutic agent(s) is/are selected from antibiotics particularly antibacterial compounds, anti-viral compounds, anti-fungals. In some embodiment, additional therapeutic agent(s) may include one or more of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGF), epidermal growth factor (EGF), platelet derived growth factor (PDGF), neu differentiation factor (NDF), hepatocyte growth factor (HGF), and hyaluronic acid.

In an aspect, according to the methods of treatment described herein, immunization is promoted by contacting the subject with a pharmaceutical composition containing an anti-hIL-6 VHH or multimeric form thereof, as described herein. Thus, methods are provided for immunization, comprising administering to a subject in need thereof, such as a subject having a disease or disorder associated with or caused by hIL-6, hIL-6 cytokine storm, dysregulation of hIL-6 signaling, or symptoms thereof, a therapeutically effective amount of a pharmaceutical composition comprising an anti-hIL-6 VHH or multimeric form thereof as active agent for a time necessary to achieve the desired result. It will be appreciated that the methods encompass protectively administering a composition comprising an anti-hIL-6 VHH or multimeric form thereof as a preventive or therapeutic measure to ameliorate, reduce, abrogate, or diminish diseases, disorders, conditions, infection or the effects thereof by hIL-6 or dysregulation of hIL-6 signaling, thus, minimizing complications associated with a slow development of immunity or response to infection (especially in compromised patients such as those who are nutritionally challenged, or at risk patients such as the elderly or infants).

A therapeutically effective dose refers to that amount of active agent which ameliorates at least one symptom or condition. Therapeutic efficacy and toxicity of active agents can be determined by standard pharmaceutical procedures in cell cultures or experimental animals, e.g., ED₅₀ (the dose that is therapeutically effective in 50% of the population) and LD₅₀ (the dose that is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices are especially useful. The data obtained from cell culture assays and from animal studies are used in formulating a range of dosages for human administration. By way of example, a therapeutic dose may be at least about 1 μg per kg, at least about 5, 10, 50, 100, 500 μg per kg, at least about 1 mg/kg, 5, 10, 50 or 100 mg/kg body weight of a composition or active component thereof per body weight of the subject, although the doses may be more or less depending on age, health status, history of prior infection, and immune status of the subject as would be known by one of skill in the art. Doses may be divided or unitary and may be administered once daily, or repeated at appropriate intervals.

### Administration of Pharmaceutical Compositions

After formulation with an appropriate pharmaceutically acceptable carrier in a desired dosage, a pharmaceutical composition comprising an anti-hIL-6 VHH or multimeric form thereof, or an anti-hIL-6 VHH or multimeric form thereof, can be administered to humans and other mammals by routes known and practiced in the art.

The administration of an anti-hIL-6 VHH or multimeric form thereof, or a pharmaceutical composition comprising of an anti-hIL-6 VHH or multimeric form thereof, as a therapeutic for the treatment or prevention of a disease, condition, infection, or pathology caused by hTL-6, hIL-6 cytokine storm, or dysregulation of hIL-6 signaling may be by any suitable means that results in a concentration of the therapeutic that, combined with other components, if desired, is effective in ameliorating, reducing, eliminating, abating, or stabilizing diseases, pathologies, disorders, or the symptoms thereof in a subject. The therapeutic may be administered systemically, for example, formulated in a pharmaceutically-acceptable composition or buffer such as physiological saline.

Routes of administration include, for example and without limitation, subcutaneous, intravenous, intraperitoneal, intramuscular, intrathecal, intraperitoneal, or intradermal injections that provide continuous, sustained levels of the therapeutic in the subject. Other routes include, without limitation, gastrointestinal, esophageal, oral, rectal, intravaginal, etc. The amount of the therapeutic to be administered varies depending upon the manner of administration, the age and body weight of the subject, and with the clinical symptoms of the bacterial infection or associated disease, pathology, or symptoms. Generally, amounts will be in the range of those used for other agents used in the treatment of disease or pathology associated with hIL-6, hIL-6 cytokine storm, or dysregulation of hIL-6 signaling, although in certain instances, lower amounts may be suitable because of the increased range of protection and treatment afforded by the described anti-hIL-6 VHHs or multimeric forms thereof as therapeutics. A composition is administered at a dosage that ameliorates, decreases, diminishes, abates, alleviates, or eliminates the effects of the hIL-6-associated disease, disorder, condition, or infection, or the symptoms thereof as determined by a method known to one skilled in the art.

In embodiments, a therapeutic or prophylactic treatment agent may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, intrathecal, or intraperitoneal) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may in some cases be formulated to release the active agent substantially immediately upon administration or at any predetermined time or time period after administration. The latter types of compositions are generally known as controlled release formulations, which include (i) formulations that create a substantially constant concentration of a therapeutic agent or drug within the body over an extended period of time; (ii) formulations that after a predetermined lag time create a substantially constant concentration of a therapeutic agent or drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the gut or gastrointestinal system; (v) formulations that allow for convenient dosing, such that doses are administered, for example, once every one or two weeks; and (vi) formulations that target a disease using carriers or chemical derivatives to deliver the therapeutic agent or drug to a particular cell type. For some applications, controlled release formulations obviate the need for frequent dosing during the day to sustain a therapeutic level in plasma, serum, or blood. In an embodiment, one or more anti-hIL-6 VHHs or multimeric forms thereof may be formulated with one or more additional components for administration to a subject in need thereof.

Any of a number of strategies can be pursued in order to obtain controlled release of a therapeutic agent in which the rate of release outweighs the rate of metabolism of the therapeutic agent or drug in question. In one example, controlled release is obtained by appropriate selection of various formulation parameters and ingredients, including, e.g., various types of controlled release compositions and coatings. Thus, the therapeutic agent or drug may be formulated with appropriate excipients into a pharmaceutical composition that, upon administration, releases the therapeutic agent or drug in a controlled manner. Examples include single or multiple unit tablet or capsule compositions, oil solutions, suspensions, emulsions, microcapsules, microspheres, molecular complexes, nanoparticles, patches, and liposomes.

Compositions for parenteral or oral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent (i.e., an anti-hIL-6 VHH or multimeric form thereof) that reduces or ameliorates a disease, pathology, or symptom thereof, associated with excess amounts, levels, or production of IL-6, or with dysregulation of IL-6 and/or IL-6 signaling, the composition may include suitable parenterally acceptable carriers and/or excipients. In some cases, an active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, compositions comprising an anti-hIL-6 VHH or multimeric form thereof are sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. In some embodiments, an anti-hIL-6 VHH or multimeric form thereof are combined, where desired, with other active substances, e.g., enzyme inhibitors, to reduce metabolic degradation. An effective amount of a pharmaceutical composition can vary according to the choice or type of anti-hIL-6 VHH or multimeric form thereof as described herein, the particular composition formulated, the mode of administration and the age, weight and physical health or overall condition of the patient, for example. In an embodiment, an effective amount of an anti-hIL-6 VHH or multimeric form thereof and/or anti-epitope tag antibody is an amount which is capable of reducing one or more symptoms of disease or pathology associated with or caused by excess amounts, levels, or production of IL-6, or with dysregulation of IL-6 and/or IL-6 signaling.

In certain embodiments, a composition includes one or more polynucleotide sequences that encode one or more anti-hIL-6 VHHs or multimeric forms thereof as described herein. In an embodiment, a polynucleotide sequence encoding an anti-hIL-6 VHH or multimeric form thereof is in the form of a DNA molecule or multimer. In some embodiments, the composition includes a plurality of nucleotide sequences each encoding an anti-hIL-6 VHH or multimeric form thereof, or any combination of anti-hIL-6 VHHs described herein, such that the anti-hIL-6 VHH antibodies or multimers thereof are expressed and produced in situ. In such compositions, a polynucleotide sequence is administered using any of a variety of delivery systems known to those of ordinary skill in the art, including eukaryotic, bacterial, or viral vector nucleic acid expression systems. Suitable nucleic acid expression systems contain appropriate nucleotide sequences operably linked for expression in a patient (such as suitable promoter and termination signals). In an embodiment, a polynucleotide molecule encoding an anti-hIL-6 VHH or multimeric form thereof can be introduced using a viral expression system or recombinant virus expression system (e.g., vaccinia or other pox virus, retrovirus, lentivirus, or adenovirus associated virus (AAV)), which uses a non-pathogenic (defective), replication competent virus. Techniques for incorporating nucleic acid (DNA) into such expression systems are well known to and practiced by those of ordinary skill in the art. The nucleic acid (DNA) can also be “naked,” as described, for example, in Ulmer et al., 1993, Science, 259:1745-1749 and as reviewed by Cohen, 1993, Science 259:1691-1692. The uptake of naked DNA can be increased by the use of nanoparticles comprising DNA or coating the DNA onto biodegradable beads, which are efficiently transported into recipient cells.

### Therapeutic Methods

Methods of treating diseases, conditions, disorders, pathologies, infections, and/or symptoms thereof associated with or caused by excess amounts, levels, or production of IL-6, or with dysregulation of IL-6 and/or IL-6 signaling are provided. Nonlimiting examples of such diseases, conditions, disorders, pathologies, infections include viral or bacterial infections; oncological diseases (cancers, carcinomas, tumors, and the like), e.g., cholangiocarcinoma, ovarian cancer, and multiple myeloma; immune-mediated diseases (autoimmune diseases and inflammatory diseases), e.g., adult rheumatoid arthritis, juvenile idiopathic arthritis, Castleman's disease, secondary amyloidosis, polymyalgia rheumatic, adult onset Still's disease, polymyositis, systemic sclerosis, large vessel vasculitis lupus erythematosus, Crohn's disease, irritable bowel disease (IBD), Sjogren's syndrome; steroid refractory Graft versus Host Disease in transplantation; type 2 diabetes, obesity and schizophrenia. The methods comprise administering a therapeutically effective amount of an anti-hIL-6 VHH or multimeric form thereof as described herein, or a pharmaceutical composition comprising these agents to a subject (e.g., a mammal such as a human). In an embodiment, the method is for treating a subject suffering from or susceptible to cytokine storm, such as occurs in conjunction with certain diseases and infections, including Covid-19 infection, as well as Adult Respiratory Distress Syndrome (ARDS). The method includes administering to the subject a therapeutically effective amount of an anti-hIL-6 VHH, multimeric form thereof, or composition thereof sufficient to treat the disease, illness, condition, disorder and/or symptom thereof, under conditions such that the disease or disorder and/or symptom thereof is treated.

The therapeutic methods include prophylactic as well as therapeutic treatment. In an embodiment, the treatment method includes administering a therapeutically effective amount of an anti-hIL-6 VHH or multimeric form thereof as described herein, or a pharmaceutical composition comprising these agents, before or during the time that a subject is administered one or more other drugs or treatments, e.g., anti-inflammatories, antibiotics, or cancer therapies. Accordingly, providing a subject with an anti-hIL-6 VHH or multimeric form of the anti-hIL-6 VHHs provides a beneficially useful and practical prophylactic and/or therapeutic treatment regimen for a subject in need.

A subject or patient includes an animal, particularly a mammal, and more particularly, a human. Such an anti-hIL-6 VHH or multimeric form thereof as described herein, or a pharmaceutical composition comprising these agents, used as therapeutics in treatments will be suitably administered to subjects or patients suffering from, having, susceptible to, or at risk for a disease, disorder, or symptom thereof, associated with or caused by infections associated with or caused by excess amounts, levels, or production of IL-6, or with dysregulation of IL-6 and/or IL-6 signaling. Determination of patients who are “susceptible” or “at risk” can be made by any objective or subjective determination obtained by the use of a diagnostic test or based upon the opinion of a patient or a health care provider (e.g., genetic test, enzyme or protein marker, family history, and the like). Identifying a subject in need of such treatment can be in the judgment of a subject himself or herself, or of a health care/medical professional and can be subjective (e.g., opinion) or objective (e.g., measurable or quantifiable by a test or diagnostic method).

### Methods of Delivery

In an embodiment, an anti-hIL-6 VHH or multimeric form thereof as described herein, or a pharmaceutical composition comprising these agents, can be administered to a subject in need of treatment for a disease, condition, disorder, pathology, infection, and/or symptoms thereof associated with or caused by excess amounts, levels, or production of IL-6, or with dysregulation of IL-6 and/or IL-6 signaling. In an embodiment, a mixture of anti-hIL-6 VHHs or multimeric forms thereof can be administered to a subject in need of treatment. In an embodiment, the anti-hIL-6 VHH or multimeric form thereof may include one or more epitope tag sequences to which anti-epitope tag antibody(ies) specifically bind. In another embodiment in which the anti-hIL-6 VHH or multimeric form thereof administered to a subject includes one or more epitope tag sequences, a specific anti-epitope tag antibody can also be administered to the subject.

In some embodiments, the administration of two or more anti-hIL-6 VHHs or multimeric forms thereof may increase the effectiveness of the therapy to treat diseases, pathologies, disorders, or infections associated with or caused by excess amounts, levels, or production of IL-6, or with dysregulation of IL-6 and/or IL-6 signaling, and reduce the severity of one or more negative symptoms related to exposure of the subject to hIL-6. In an embodiment, administering to a subject the anti-hIL-6 VHH or multimeric form thereof that includes one or more, e.g., two, epitope tag sequences may result in improved therapy, treatment, or protection against diseases, pathologies, disorders, or infections associated with or caused by excess amounts, levels, or production of IL-6, or with dysregulation of IL-6 and/or IL-6 signaling. In an embodiment, the epitope tag sites of the anti-hIL-6 VHH or multimeric form thereof are bound by a specific anti-tag antibody. The administration of an anti-hIL-6 VHH or multimeric form thereof as described herein, or a composition comprising the agent, and the administration of one or more anti-epitope tag antibodies may be performed simultaneously or sequentially in time. In an embodiment, an anti-hIL-6 VHH or multimeric form thereof is administered before, after, or at the same time as the administration of another anti-hIL-6 VHH or multimeric form thereof, or before administration of an anti-tag antibody, provided that the anti-hIL-6 VHH(s) or multimeric form(s) thereof and/or the anti-tag antibody(ies) are administered close enough in time to have the desired effect (e.g., before the anti-hIL-6 VHHs or multimeric forms thereof have been cleared by the body). Accordingly, “co-administration” embraces the administration of an anti-hIL-6 VHH or multimeric form thereof and a subsequent anti-hIL-6 VHH or multimeric form thereof, or the anti-tag antibody, at time points that will achieve effective treatment of diseases, pathologies, disorders, or infections associated with or caused by excess amounts, levels, or production of IL-6, or with dysregulation of IL-6 and/or IL-6 signaling, or reduce the severity thereof. The described methods are not limited by time intervals between which an anti-hIL-6 VHH or multimeric form thereof and/or the anti-tag antibody(ies) are administered; provided that these agents, or compositions containing these agents, are administered close enough in time to produce or achieve the desired effect. In an embodiment, only an anti-hIL-6 VHH or multimeric form thereof is administered to a subject in need thereof. In another embodiment, an anti-hIL-6 VHH or multimeric form thereof and an anti-epitope tag antibody are premixed and administered together, or are not premixed but are co-administered within minutes of each other. In other embodiments, the anti-hIL-6 VHH or multimeric form thereof and anti-epitope tag antibody(ies) are co-administered with other medications, drugs, compounds, or compositions suitable for treating the disease, disorder, pathology, condition, and the like.

In yet other embodiments, an anti-hIL-6 VHH or multimeric form thereof, or a composition containing the agent(s), is administered to a subject prior to the potential risk of diseases, pathologies, disorders, or infections associated with or caused by excess amounts, levels, or production of IL-6, or with dysregulation of IL-6 and/or IL-6 signaling afflicting the subject, in order to protect the subject from these medical afflictions and the symptoms thereof. For example, an anti-hIL-6 VHH or multimeric form thereof and/or anti-epitope tag antibody (“clearing antibody”) is administered minutes, hours or days prior to the risk of a subject's contracting or presenting with a disease, pathology, condition, disorder, or infection associated with or caused by excess amounts, levels, or production of IL-6, or with dysregulation of IL-6 and/or IL-6 signaling. Alternatively, an anti-hIL-6 VHH or multimeric form thereof is administered concomitantly with the risk of disease, etc., exposure of a subject or slightly after the risk of disease, etc., exposure.

Administration of an anti-hIL-6 VHH antibody or multimeric form thereof ameliorates, reduces, or alleviates the severity of diseases, etc., as described herein, or one or more of the symptoms of the diseases, etc. The presence, absence, or severity of symptoms is measured, for example, using physical examination, tests and diagnostic procedures known and practiced in the art. In certain embodiments, the presence, absence and/or level of hIL-6 cytokine are measured using methods known and employed in the art. Symptoms or levels of the hIL-6 cytokine can be measured at one or more time points (e.g., before, during and after treatment, or any combination thereof) during the course of treatment with an anti-hIL-6 VHH or multimeric form thereof to determine if the treatment is effective. A decrease, reduction, or no change in the levels of the hIL-6 cytokine, or in the severity of symptoms associated with hIL-6-induced disease, etc., indicates that treatment is effective, and an increase in the level of hIL-6 or in the severity of symptoms in a subject indicates that treatment is not effective. In various embodiments, the symptoms and levels of hIL-6 are measured using methods known and employed in the art. Methods, compositions and kits involving the use of the anti-hIL-6 VHHs or multimeric forms thereof described herein decrease and alleviate the symptoms of hIL-6-induced disease, etc., and also improve survival from cytokine storm or residual disease caused by or associated with hIL-6-induced diseases, etc.

In some embodiments, encapsulation and enteric coating techniques and processes commonly known and used in the art are suitable for delivering anti-hIL-6 VHHs antibodies to subjects. In embodiments, nanoparticle-based delivery of drugs and biologics and enteric coating of nanoparticles have been described by J. K. Patra et al., 2018, J. Nanobiotech, 16, Art. No. 71 (doi.org/10.1186/s12951-018-0392-8); US Publication No. 20200129444, the contents of which are incorporated by reference herein. Nanoparticles engineered to deliver hIL-6-binding and/or neutralizing VHHs may be introduced into a subject in need thereof.

In an embodiment, a polynucleotide encoding an anti-hIL-6 VHH antibody or multimeric form thereof as described herein, constitutes mRNA. In an embodiment the mRNA is a formulated mRNA, namely, mRNA that is packaged by a formulant, material, or biomaterial (e.g., as a delivery agent) to protect the mRNA from degradation and to facilitate its entry into cells in the body for expression of the encoded anti-hIL-6 VHH antibody protein or therapeutic protein. A wide variety of mRNA formulants or delivery agents may be used, for example, without limitation, ionizable lipids; biodegradable ionizable lipids (e.g., ATX-100, LP-01, OF-02, Lipid 5); polymeric materials (e.g., polyethyleneimines (PEIs), poly(glycoamidoamine) polymers or poly(glycoamidoamine) polymers modified with fatty chains, poly(β-amino)esters (PBAEs), or polymethacrylates); dendrimers (e.g., polyamidoamine (PAMAM) or polypropylenimine-based dendrimers, PAMAM (generation 0) dendrimer co-formulated with poly(lactic-co-glycolic acid) (PLGA) and ceramide-PEG); cell penetrating peptides; and cationic or zwitterionic lipids, e.g., as described in P. S. Kowalski et al., 2019, Mol. Ther., 27(4):710-728, the contents of which are incorporated by reference herein.

In an embodiment, a polynucleotide encoding an anti-hIL-6 VHH antibody or multimeric form thereof as described herein, in particular, mRNA, in the form of nanoparticles, such as lipid nanoparticles, may be used to deliver these anti-hIL-6 binding agents and produce effective and long-lasting antibody titers in subjects who are administered (immunized with) the mRNA-nanoparticles. In a particular embodiment, the mRNA, which is otherwise unmodified, may be codon optimized to afford efficient expression of an anti-hIL-6 VHH or multimeric form thereof from the transcribed mRNA. It has been reported that exogenous mRNA has the ability to instruct cells to produce VHHs, as well as other types of antibodies. See, M. Thran et al., 2017, EMBO Mol. Medicine, online publication no. DOI 10.15252/emmm.201707678. The advantages of using mRNA for passive immunization are appreciated by those in the art. (See, M. Thran et al., Id.). mRNA-based approaches for therapeutics may be safer and more cost effective compared with DNA-based approaches. Because mRNA does not integrate into a host's DNA and is more transient in nature, mRNA-based protein expression is considered to be easier to control for protein expression.

### Substantially Identical Amino Acid and Nucleotide Sequences for VHHs

There is a large body of information in the literature supporting the fact that closely related antibody (Ab) sequences are capable of performing the same binding and therapeutic functions such that this is now generally accepted by those with ordinary skill in the art of immunological sciences. The creation of Abs with small numbers of amino acid sequence variations occurs naturally within mammals and some other animal species during the process of ‘affinity maturation’ in which Ab-producing cells that bind a newly encountered antigen (Ag) are expanded, and their progeny cells contain random mutations within portions of the Ab coding DNA that results in new, related Ab sequences. The cells expressing Abs that have gained improved binding properties for the new Ag are then selected and expanded, thereby increasing the amount of the improved antibody in the animal. This process continues through multiple generations of mutation and selection until Abs with greatly improved antigen binding properties result. The process of Ab affinity maturation demonstrates that related, yet not identical, Ab amino acid sequences can possess similar target binding properties and perform similar therapeutic functions in vivo.

Example 1 herein provides anti-hIL-6 VHH antibodies having related sequences that perform similar functions and provide similar therapeutic benefits. The Abs described herein are heavy-chain only, single domain VHH antibodies, which are generated in camelid alpacas, which have been reported to be convenient sources of camelid VHH antibodies (See, e.g., Maass, D. R. et al., 2007, J. Immunol. Methods, 324:13-25). Briefly, alpacas are immunized with a selected hIL-6 antigen (hTL-6 Ag) multiple times to permit the animal to undergo affinity maturation of the anti-hIL-6 VHHs that are produced. Anti-hIL-6 VHHs are then isolated and the encoding DNA selected for expression of soluble VHHs that bind hIL-6 Ag and have potential therapeutic or diagnostic properties. During this process, many examples of closely related anti-hIL-6 binding VHHs are isolated, which are distinctive, and which are presumably intermediates that result from the affinity maturation process which occurs during anti-hIL-6 VHH production in alpaca lymphocytes. These related anti-hIL-6 VHHs are screened for binding to hIL-6 Ag, and the most promising members of homology groups of hIL-6-binding VHHs are identified and become lead candidates for further development.

Similar to all mammalian antibodies, VHHs consist of four, well-conserved ‘framework’ regions (FRs) which are important in forming the antibody structure. Between the FRs (FR1, FR2, FR3 and FR4) are three much less well-conserved CDRs or hypervariable regions (CDR1, CDR2 and CDR3) which principally interact with and bind to antigenic determinants or epitopes on antigens (Ags), such as hIL-6. The CDR sequences vary widely so as to interact and bind to epitopes of Ags. The third CDR, CDR3, is generally the longest in sequence and is most diverse of the CDRs within VHHs, both in size and sequence. By way of nonlimiting example, CDR3 in VHHs can range in size from about 7 to about 28 amino acid residues. The CDR3 regions of VHHs generated in the same alpacas and selected for binding to a common target Ag are highly similar in size (number of amino acids comprising CDR3) and can vary in their amino acid identities. Without intending to be bound by theory, VHHs and CDR3 regions that bind to the same hIL-6 target Ag are considered to have resulted from affinity maturation of a common precursor VHH within the animal and are classified as a ‘homology group.’ Individual VHHs within a homology group are classified by their binding to the target Ag, and the members of the VHH homology group are able to ‘compete’ with each other for binding to the Ag, thus demonstrating that they bind to the same region on the target Ag. In VHH molecules, the CDRs (CDR1, CDR2 and CD3) play a role in the ability of a VHH to bind to the target Ag, e.g., hIL-6, in conjunction with CDR1 and CDR2.

Since the FRs maintain the structure of a VHH and the positioning of the CDRs for binding to the target Ag, the FRs of VHHs typically do not vary extensively in sequence. (FIG. 9). However, some VHH FR amino acid sequence variation is permissible, particularly in cases in which an amino acid substitution involves the replacement or substitution of one amino acid with another amino acid having similar properties (e.g., similarity in being charged or uncharged), i.e., a conservative substitution. Such conservative changes in FRs can often be found naturally within VHHs that have undergone affinity maturation in an animal. Similar to the case with FRs, VHH CDRs also typically do not vary extensively in amino acid sequence or type so as not to compromise their ability to specifically bind to Ag. As would be appreciated by one skilled in the art, an estimation of the extent of amino acid sequence variation that can be tolerated within VHHs without compromising their Ag binding ability can be made by observing the variation that occurs naturally within affinity-matured homology groups of VHHs isolated from the same types of animals and which bind to the same Ag.

In an embodiment, sequence variation is particularly acceptable in the CDR regions, e.g., CDR1, CDR2, and/or CDR3, while the feature of VHH binding to antigen hIL-6 is maintained. In an embodiment, amino acid sequence variation results from conservative amino acid substitutions in a VHH sequence. In an embodiment, the conservative amino acid substitutions are in one or more CDR sequences of the VHH polypeptide. In an embodiment, the conservative amino acid substitutions are in one or more FR sequences of the VHH polypeptide. In an embodiment, the conservative amino acid substitutions are in one or more CDR sequences and in one or more FR sequences of the VHH polypeptide.

An example evidencing that VHH sequence variation is acceptable within related VHHs having the same Ag binding characteristics is described in Tremblay et al., 2013, Infect Immun 81:4592-4603. In this report, 11 VHH sequences comprise a large homology group with closely related CDR3 sequences, and the unusual property of cross-specific binding to two different Shiga toxins, Stx1 and Stx2. Two of the more distantly related VHH members of this homology group are characterized as having common Ag binding characteristics. These two related VHHs were found to have 32 amino acid changes in the total VHH sequence of 120 or 121 residues. Thus, a 26% variation in amino acid sequence did not adversely affect the common Ag binding properties of the VHH proteins.

### Kits

Provided herein are kits for the treatment or prevention of an infection, condition, disorder, disease, or pathology, and/or the symptoms thereof, caused by or associated with hIL-6 and/or its functional activity, or the aberrant or dysfunctional activity of hIL-6. In some embodiments, the kit includes an effective amount of one or more anti-hIL-6 VHHs or multimeric forms thereof as described herein, in unit dosage form. In an embodiment, the kit further contains an anti-epitope tag antibody, in unit dosage form. In other embodiments, the kit includes a therapeutic or prophylactic composition containing an effective amount of one or more anti-hIL-6 VHHs or multimeric forms thereof, in unit dosage form. In still other embodiments, the kit includes a therapeutic or prophylactic composition containing an effective amount of one or more anti-hIL-6 VHHs or multimeric forms thereof, and an anti-epitope tag antibody, in unit dosage form. In some embodiments, the kit comprises a device, e.g., an automated or implantable device for subcutaneous delivery; an implantable drug-eluting device, or a nebulizer or metered-dose inhaler, for dispersal of the composition or a sterile container which contains a pharmaceutical composition. Non-limiting examples of containers include boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition of the invention is provided together with instructions for administering the pharmaceutical composition containing one or more anti-hIL-6 VHHs or multimeric forms thereof, or one or more anti-hIL-6 VHHs or multimeric forms thereof and an anti-epitope tag antibody, to a subject having or at risk of contracting or developing an infection, condition, disorder, disease, or pathology, and/or the symptoms thereof, caused by or associated with hIL-6 and/or its functional activity, or the aberrant or dysfunctional activity of hIL-6. The instructions will generally include information about the use of the composition for the treatment or prevention of an infection, condition, disorder, disease or pathology, and/or the symptoms thereof, caused by or associated with hIL-6 and/or its functional activity, or the aberrant or dysfunctional activity of hIL-6. In other embodiments, the instructions include at least one of the following: description of the therapeutic/prophylactic agent; dosage schedule and administration for treatment or prevention of infection, disease or symptoms thereof caused by or associated with hIL-6 or its dysfunctional activity; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

In another aspect, a kit is provided for treating a subject having, at risk of, or susceptible to having an infection, condition, disorder, disease, or pathology, and/or the symptoms thereof, caused by or associated with hTL-6 and/or its functional activity, or the aberrant or dysfunctional activity of hTL-6, in which the kit includes a pharmaceutical composition for treating the subject, and the pharmaceutical composition includes at least one recombinant anti-hIL-6 VHH or multimeric form thereof. In an embodiment, the anti-hIL-6 VHH or multimeric form thereof neutralizes hIL-6 activity, thereby treating the subject; a container; and, instructions for use. In various embodiments, the instructions for use include instructions for a method for treating a subject having, at risk of, or susceptible to having an infection, condition, disorder, disease, or pathology, and/or the symptoms thereof, caused by or associated with hIL-6 and/or its functional activity, or the aberrant or dysfunctional activity of hIL-6 using the kit comprising the pharmaceutical composition.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the Examples that follow.

### EXAMPLES

### Example 1—Anti-IL-6-Binding VHHs

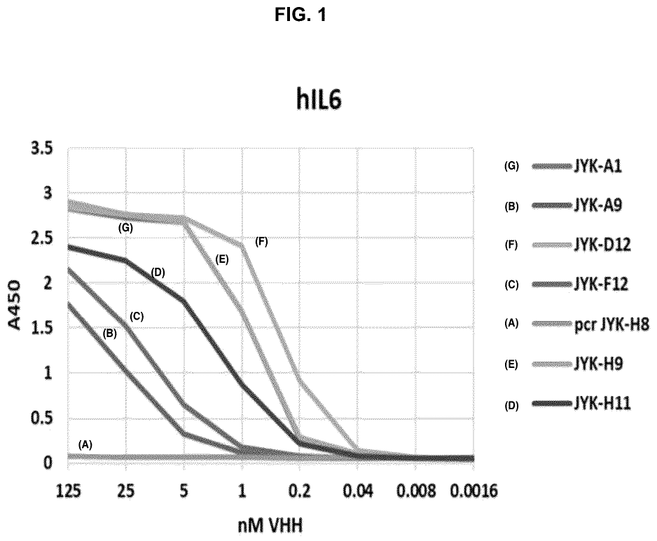

Presented in Example 1 are the amino acid and encoding polynucleotide (nucleic acid) sequences of human IL-6 binding VHH polypeptides (anti-hIL-6 VHHs) as described herein. FIGS. 1(a)-(d) depict the overall structure of a single domain VHH polypeptide compared with that of a classical immunoglobulin molecule. The amino acids comprising the Complementarity Determining Regions (CDRs) of each of the anti-IL-6 VHHs are designated in each VHH polypeptide as follows: CDR1 is designated by a single underline; CDR2 is designated by a double underline; and CDR3 is designated in bold with a single underline.

Human IL6-binding VHHs:
JYK-A1 VHH amino acid sequence (JYR-1 expression plasmid)
(SEQ ID NO: 1)
QLQLAETGGGLVQPGGSLRLSCAASGFTLDYYAVGWFRQAPGKEREGISCISSSDLKTYYAD
SVKGRFTISRDYAKSTVSLQMNSLKPEDTGVYYCAAGTWDLKFGYNISACVGSYEYDYWDQG
TQVTVSS
JYK-A1 VHH polynucleotide sequence
(SEQ ID NO: 2)
cagttgcagctggcggagactggtggagggttggtccagcctggggggtctctgagactctc
ctgtgcagcctctggattcactttggattattatgccgtaggctggttccgccaggccccag
ggaaggagcgtgaggggatctcatgtattagtagtagtgatcttaaaacatactatgcagac
tccgtgaagggccgattcaccatctccagagactacgccaagagcacggtgtctctgcaaat
gaacagcctgaaacctgaggacacaggcgtttattactgtgcggcgggcacatgggatctta
agttcggctataatattagtgcctgcgtgggatcttatgagtatgactactgggaccagggg
acccaggtcaccgtctcctca
JYK-A9 VHH amino acid sequence (JYR-2 expression plasmid)
(SEQ ID NO: 3)
QVQLVESGGGLVQAGDSLTLSCAASGRPFSSFAMGWFRQAPGKEREFVAAITWSRGTTHYAD
SVKGRFTISGDNAKNTVFLQMNSLKPEDTAVYYCAAADGWKVVSTASPAYDYWGQGTQVTVS
S
JYK-A9 VHH polynucleotide sequence
(SEQ ID NO: 4)
caggtgcagctcgtggagtcaggaggaggattggtgcaggctggggactctctgacactctc
ctgtgcagcctctggacgccccttcagtagttttgccatgggctggttccgccaggctccag
ggaaggagcgtgagtttgtagcagctattacatggagtcgtggtaccacacactatgccgac
tccgtgaagggccggttcaccatctccggggacaacgccaagaacacggtgtttctgcaaat
gaacagcctaaaacctgaggatacggccgtttattactgtgcagcagcggatggatggaagg
tagttagtactgctagccccgcgtatgactactggggccaggggacccaggtcaccgtctcc
tca
JYK-D12 VHH amino acid sequence (JYR-3 expression plasmid)
(SEQ ID NO: 5)
QLQLVESGGGLVQPGGSLGLSCAASGFTLAYYGIGWFRQAPGKEREGVACISSSDLSTYYAD
SVKGRFTISRDNAKDTVYLQMNSLKPEDTAVYYCAAGTWDLKFGYSRSNCVRSYEYDYWGQG
TQVTVSS
JYK-D12 VHH polynucleotide sequence
(SEQ ID NO: 6)
cagttgcagctggtggagtccggtggaggcttggtgcagcctggggggtctctgggactctc
ctgtgcagcctctggattcactttggcttattatggcataggctggttccgccaggccccag
ggaaggagcgtgagggggtcgcatgtattagtagtagtgatcttagcacatactatgcagac
tccgtgaagggccgattcaccatctccagagacaacgccaaggacacggtgtatctgcaaat
gaacagcctgaaacctgaggacacagccgtttattactgtgcagcgggcacatgggatctta
aattcggctatagtagaagtaactgcgtgcgatcttatgagtatgactactggggccagggg
acccaggtcaccgtctcctca
JYK-F12 amino acid sequence (JYR-4 expression plasmid)
(SEQ ID NO: 7)
QVQLAETGGGSVQAGGSLTLSCAASGRTFSSRAMGWFRQAPGKEREFVAVISWTGSPYYTDS
VKGRFTISRDDAKNTVYLQMNSLKPEDTAVYYCAATSEHVMLVVTTRGGYDYWGQGTQVTVS
S
JYK-F12 polynucleotide sequence
(SEQ ID NO: 8)
caggtgcagctggcggagaccggcggaggatcggtgcaggctgggggctctctgacactctc
ctgtgcagcctctggacgcaccttcagtagcagagccatgggctggttccgccaggctccag
ggaaggagcgtgagtttgtagcagttattagctggactggtagcccatactatacagactcc
gtgaagggccgattcaccatctccagagacgacgccaagaacacggtgtatctgcaaatgaa
cagcctgaaacctgaggacacggccgtttattactgcgcagcgacgtcagaacatgtaatgc
tggtagttactacgcgtgggggtatgactactggggccaggggacccaggtcaccgtctcc
tca
JYK-H9 VHH amino acid sequence (JYR-6 expression plasmid)
(SEQ ID NO: 9)
QLQLVETGGGLVQPGGSLRLSCAASGFTLDYYAIGWFRQAPGKEREGVSCLSSSDRSTYYVD
SVKGRFTISRDDDKNTAYLQMNSLKPEDTATYYCAAGTWDLKWGYNISACVGSYEYDYWGQG
TQVTVSS
JYK-H9 polynucleotide sequence
(SEQ ID NO: 10)
cagttgcagctggtggagacaggaggaggcttggtgcagcctggggggtctctgagactctc
ctgtgcagcctctggattcactttggattattatgccataggctggttccgccaggctccag
ggaaggagcgtgagggggtctcatgtttgagtagtagtgatcgtagcacatactatgtagac
tccgtgaagggccgattcaccatctccagagacgatgacaagaacacggcgtatctgcagat
gaacagcctgaaacctgaggacacagccacttattactgtgcagcgggcacatgggatctta
aatggggctataacattagtgcctgcgtgggatcttatgagtatgactactggggccagggg
acgcaggtcaccgtctcctca

### Example 2—VHH-Display Library Preparation from Genes Expressed in Immunized Camelids (Alpacas) and ELISA Analysis

In general, two alpacas were immunized with human IL-6 protein (hIL-6), (100 μg), by successive multi-site subcutaneous (SC) injections at three week intervals. For the first immunization, the adjuvant was alum/CpG and subsequent immunizations used alum. All alpacas achieved ELISA anti-IL-6 titers of 1:100,000. Blood was obtained from the alpacas for peripheral blood lymphocyte (PBL) preparation seven days after the final immunization, and RNA was extracted using the RNEASY kit (Qiagen, Valencia, CA). cDNA and anti-hIL-6 VHH-display phage libraries were prepared. By way of example, VHH libraries are prepared as described in Methods in Molecular Biology, “Single Domain Antibodies—Methods and Protocols,” Eds. D. Saerens and S. Muyldermans, Humana Press (Springer), 2012; in E. Romao et al., 2018, Methods in Molecular Biology, “Phage Display: Methods and Protocols, Eds. M. Hust and T. Soon Lim, Springer Science and Business Media, Vol. 1701, pages 169-187, 2018; and in E. Pardon et al., Nature Protocols, Vol. 9(3):674-693, 2014, the contents of which are incorporated herein by reference. Affinity panning of the library and binding assays (e.g., ELISA assays) were carried out to identify VHH antibody clones (e.g., VHH monomers) that bound to hIL-6 The top scoring clones for binding to hIL6 (those with the highest affinity binding to hIL-6) were subjected to DNA fingerprinting analysis using standard methods, and the VHH coding DNAs from clones displaying unique fingerprints were sequenced.

Based on sequence analysis, five clonally-independent families of the anti-hIL-6 VHHs, i.e., having sequence homology derived from independent B cell origins, were obtained. One family represented the majority of VHH family members that strongly bound to hIL-6 as determined by ELISA; therefore, multiple variants of this family were also sequenced. The anti-hIL-6 VHH coding DNA of selected VHHs was re-cloned into E. coli expression vectors and the anti-hIL-6 VHH proteins were expressed and purified using standard methods. The purified anti-hIL-6 VHHs were subjected to dilution ELISA analysis to assess their apparent affinities for binding to hIL-6. Anti-hIL-6 VHH JYK-D12 demonstrated the highest apparent affinity for binding to plate-coated hIL6 using a panning technique. (See, e.g., Mukherjee, J., et al., 2012 PLoS ONE, 7, e299411). The EC₅₀ value of JYK-D12 for binding hTL-6 as determined by ELISA was about 0.4 nM. The results of a representative dilution ELISA analysis are shown in FIG. 1 and FIG. 3A.

Cell-based assays showed that three, closely related anti-hTL-6 VHHs, namely, JYK-A1, JYK-D12 and JYK-H9, all displayed highly potent hIL6-neutralization properties. Further screening of anti-hIL-6 VHH-display library was performed, and numerous additional anti-hIL-6 VHHs in the same family of VHHs (called “the JYK-D12 family”) were identified (FIG. 4). An alignment of the amino acid sequences of 15 anti-hIL-6 VHH members of the JYK-D12 family that were identified in the single re-screening are shown in FIG. 2. These results indicate that significant amino acid sequence flexibility may exist among the anti-hIL-6 VHH family members, even within their CDRs, yet the anti-hIL-6 VHH antibodies are able to bind to hIL-6 used as immunogen.

In some embodiments, an anti-hIL-6 VHH antibody (e.g., JYK-D12) that demonstrated potent hIL-6 binding and/or neutralization properties (or a polynucleotide encoding the anti-hIL-6 VHH antibody) was expressed as a dimer, e.g., a homodimer, in which the anti-hIL-6 VHH components of the dimer molecule were separated by a long or a short amino acid spacer or linker (Example 7).

In summary, several hundred individual clones were screened for hIL-6-binding; positive clones were characterized; and the new and unique hIL-6-binding VHH coding DNAs were re-cloned into an expression vector for soluble protein expression and purification. The purified, quantified VHHs were characterized for binding affinities for hIL-6 and, in some cases, for their potencies to neutralize hIL-6 in both in vitro cell-based assays and in in vivo animal studies.

### Example 3—In Vitro Efficacy of Anti-Human IL-6 (hIL-6) VHH Antibodies

hIL-6 binding assays (ELISAs) and cell proliferation (neutralization) assays were performed using representative anti-hIL-6 VHH antibodies and a dimer thereof as described herein. The results of the hIL-6 binding assay are presented in FIGS. 3A and 3B. The results of the cell proliferation assays are presented in FIG. 4.

The cell proliferation (neutralization) assay involved the use of hIL-6 and 7TD1 cells. All samples were run in parallel. The samples included the anti-hIL-6 VHH antibodies shown in FIG. 4, as well as others not shown, and human IL-6, which was reconstituted prior to use in water and stored at −20° C. in 1× phosphate buffered saline (PBS)+1% bovine serum albumin (BSA). The 7TD1 cells were resuspended in assay medium containing 10% calf serum (CS) and 2× Gentamicin. The cells were transferred to wells in a multi-well tissue culture plate (100 μl culture/well), (8,000 cells/well; Passage #5). The hIL-6 cytokine was serially diluted in assay medium in a separate tissue culture plate. 100 μl of the diluted cytokine was added to the cells in the assay plate. The final assay volume of each well in the plate was 200 μl; the assay medium contained 10% CS, 2× Gentamicin and hIL-6 at dilutions of 0.4000 ng/ml, 0.1000 ng/ml, 0.0250 ng/ml, 0.0063 ng/ml, 0.0016 ng/ml and 0.0004 ng/ml. The cells were incubated with the cytokine for 67 hours to 3 days. Thereafter, 20 μl of Promega substrate (CellTiter 96 Aqueous On Solution Reagent) was added to each well. Following incubation at 37° C., the wells of the plate were read at OD490 nm to measure cell proliferation. After 5 hours, the average minimum OD (0.00-0.0004 ng/ml) was 1.190; the average maximum net OD (0.0250-0.40 ng/ml) was 0.268; for 3 assays, the calculated net OD 490 nm for ED50 was 0.134, 0.147, and 0.164. The ED50 was 0.004-0.006 ng/ml hIL-6 using 7TD1 cells.

A summary of the neutralization data is as follows: for hIL-6-binding VHH JYK-A1, complete neutralization was determined at an ED50 of 0.0247 μg/ml), (ED50 of 0.007-0.010 g/ml using 7TD1 cells); for hIL-6-binding VHH JYK-A9, complete neutralization was determined at an ED50 of 2.0 μg/ml), (ED50 of 0.42-0.62 μg/ml using 7TD1 cells); for hIL-6-binding VHH JYK-D12, complete neutralization was determined at an ED50 of 0.0009 μg/ml), (ED50 of <0.0003 μg/ml using 7TD1 cells); for hIL-6-binding VHH JYK-F12 (NB4), partial neutralization was determined, (ED50 of 0.61-0.92 μg/ml using 7TD1 cells); hIL-6-binding VHH JYK-H8 was determined to have low or no biological activity; for hIL-6-binding VHH JYK-H9, complete neutralization was determined at an ED50 of 0.0027 μg/ml), (ED50 of 0.0003-0.0005 μg/ml using 7TD1 cells); for hIL-6-binding VHH JYK-H11, complete neutralization was determined at an ED50 of 0.0667 μg/ml), (ED50 of 0.17-0.25 μg/ml using 7TD1 cells). hIL-6-binding VHH JYK-A1, hIL-6-binding VHH JYK-D12, and hIL-6-binding VHH JYK-H9 showed potent neutralization activity.

### Example 4—Anti-hIL-6 VHH Antibodies have Neutralizing Activity In Vitro

Neutralization assays were performed using the anti-hIL-6 VHH antibodies as described herein to determine their ability to abolish JAK-STAT signaling in vitro. Briefly, HEK293 cells were plated in tissue culture plates to greater than 80% confluence. The cells were transfected with a STAT3-luciferase reporter polynucleotide for 24 hours prior to being treated for 6 hours with either hIL-6 (50 ng) or with hIL-6 (50 ng) plus an anti-hIL-6 VHH antibody (100 ng). In these assays, a homodimer of hIL-6-binding VHH JYK-D12 was used. In embodiments, a closely related VHH polypeptide (e.g., as presented in Table 1) may be used. Luciferase activity was assessed after 6 hours. The hIL-6 (50 ng) plus anti-hIL-6 VHH antibody (100 ng) were incubated for 1 hour at 4° C. prior to addition to the cells. FIG. 5 demonstrates that the anti-hIL-6 VHH antibody abolished JAK-STAT signalling.

### Example 5—Anti-hIL-6 VHH Antibodies have Neutralizing Activity In Vivo

The experiments described in this Example were conducted to assess the ability of a representative anti-hIL-6 VHH antibody as described herein to inhibit hepatic STAT3 activation induced by hIL-6 in vivo.). In particular, a homodimer of the hIL-6-binding VHH JYK-D12 was used.

The Janus family of tyrosine kinases (JAK) and the signal transducer and activator of transcription (STAT) family is a major signaling pathway involved in cellular metabolism. The JAK/STAT signaling pathway is also involved in several cellular processes, such as proliferation, apoptosis, differentiation and migration. Dysregulation of the JAK/STAT signaling pathway is associated with a wide range of leukemias, lymphomas, head & neck cancers, melanomas and breast cancers. STAT proteins are activated by cytokines (e.g., IL-6, hIL-6) and other receptor kinases, including EGFR, FGFR, CSF1R, PDGFR and other G-protein coupled receptors. The transcription factor and receptor are often expressed at very low levels.

Therefore, an assay with high sensitivity and specificity is beneficial. The RNAscope assay (RNAScope 2.0 HD, Advanced Cell Diagnostics, Inc. (ACD)) provides a method for detecting mRNA transcription targets. Other assays are available for carrying out JAK-STAT signaling assays, e.g., the Human Magnetic Luminex Assay and ELISA assays (R&D Systems; Bio-Techne Corp.).

### In Vivo Neutralization Efficacy of Anti-hIL-6 VHH Antibody

Experiments were performed to assess the in vivo efficacy of an anti-hIL-6 VHH antibody administered to mice. One group of C57BL/6 mice was injected intraperitoneally with 1 μg hIL-6 in PBS and a second group of C57BL/6 mice was injected with 1 μg hIL-6 plus the representative anti-hIL-6 VHH antibody (4 μg). The animals were euthanized 30 minutes post-injection, their livers were isolated; and protein was extracted. Western blot analyses were carried out to assess the phosphorylation of STAT3 using P-STAT3 Y705 antibody (1:1,000 dilution) and STAT3 antibody (1:2,000 dilution), (Cell Signaling Technology (CST), Danvers, MA). The results of the Western blot analysis in which the phosphorylation of STAT3 is abolished in vivo by the representative anti-hIL-6 VHH antibody (4 μg) and hIL-6 (1 μg) are shown in FIG. 6A.

### In Vivo Neutralization Efficacy of Anti-hIL-6 VHH Antibody in a Dose Response Analysis

In vivo dose-response experiments were conducted to assess the neutralizing activity of the representative anti-hIL-6 VHH antibody. In the experiments, one group of C57BL/6 mice was injected intraperitoneally with 0 or 1 μg hIL-6 in PBS and other groups of C57BL/6 mice were injected with 1 μg hIL-6 plus different doses of the anti-hIL-6 VHH antibody, i.e., 4 μg, 1 g, 0.5 μg, 0.25 μg, and 0 μg doses. The animals were euthanized 30 minutes post-injection; their livers were isolated; and protein was extracted. Western blot analyses were carried out to assess the phosphorylation of STAT3 using P-STAT3 Y705 antibody (1:1,000 dilution) and STAT3 antibody (1:2,000 dilution), (Cell Signaling Technology (CST), Danvers, MA). The results of the Western blot analysis to assess the dose response of the representative anti-hIL-6 VHH antibody are shown in FIG. 6B, which demonstrates that the phosphorylation of STAT3 is abolished in vivo by the anti-hIL-6 VHH antibody at doses of 0.25, 1 and 4 μg, and hIL-6 (1 μg).

### In Vivo Neutralization Efficacy of Anti-hIL-6 VHH Antibody Versus an Anti-IL-6R Antibody

Experiments were conducted to assess the use of the representative anti-hIL-6 VHH antibody versus an antibody directed against the interleukin-6 receptor (IL-6R), i.e., Tocilizumab (ACTEMRA, Genentech, Inc.), in vivo. In this experiment, one group of C57BL/6 mice was injected intraperitoneally with 0 or 1 μg hIL-6 in PBS; another group of C57BL/6 mice were injected with 0.5 μg hTL-6 plus 0.5 μg anti-hTL-6 VHH antibody; and additional groups of C57BL/6 mice were injected with 1 μg hIL-6 plus different doses the anti-hTL-6R antibody Tocilizumab, i.e., 500 μg, 50 μg, 5 μg, 0.5 μg, and 0 μg doses. The animals were euthanized 30 minutes post-injection; their livers were isolated; and protein was extracted. Western blot analyses were carried out to assess the phosphorylation of STAT3 using P-STAT3 Y705 antibody (1:1,000 dilution) and STAT3 antibody (1:2,000 dilution), (Cell Signaling Technology (CST), Danvers, MA). The results of the Western blot analysis to assess in vivo neutralization activity of the anti-hIL-6 VHH antibody versus that of the anti-hTL-6R antibody are shown in FIG. 6C. FIG. 6C demonstrates that the phosphorylation of STAT3 was abolished in vivo by the anti-hTL-6 VHH antibody at a dose of 0.5 μg, while STAT3 phosphorylation was abolished by 500 μg of the anti-hIL-6R antibody Tocilizumab. The representative anti-hIL-6 VHH antibody used in the in vivo neutralization assay was found to be effective at a 1000-fold lower dose than Tocilizumab, thus evidencing the significant neutralization potency of the anti-hIL-6 VHH antibodies described herein.

### Example 6—Anti-hIL-6 VHH Antibody Cross-Reacts with Mouse IL-6 In Vivo

Experiments were conducted to determine whether the representative anti-hIL-6 VHH antibody cross-reacted with mouse IL-6 in vivo. In this experiment, one group of C57BL/6 mice was injected intraperitoneally with 1 μg of mouse IL-6 (mIL-6) in PBS; another group of C57BL/6 mice was injected with 1 μg of mouse IL-6 (mIL-6) plus 0.4 μg anti-hIL-6 VHH antibody. The animals were euthanized 30 minutes post-injection; their livers were isolated; and protein was extracted. Western blot analyses were carried out to assess the phosphorylation of STAT3 using P-STAT3 Y705 antibody (1:1,000 dilution) and STAT3 antibody (1:2,000 dilution), (Cell Signaling Technology (CST), Danvers, MA). The results of the Western blot analysis to assess the cross-reactivity of the anti-hIL-6 VHH antibody in mIL-6-induced hepatic STAT activation are shown in FIG. 7. FIG. 7 demonstrates that the anti-hIL-6 VHH antibody affected mIL-6-induced hepatic STAT activation by abolishing the phosphorylation of STAT3 in vivo at a dose of 4 μg.

The results of the experiments described in the examples above demonstrate that the anti-hIL-6 VHH antibodies described herein efficiently and effectively inhibit IL-6—(e.g., human and mouse IL-6) induced STAT3 activation in liver. The described single domain, anti-hIL-6 VHH antibodies may be useful as treatments and therapeutics in patients having dysfunctional, abnormal, or aberrant IL-6 signaling, and/or in patients experiencing hIL-6 cytokine storm (CS), for example, acutely ill patients with SARS-Covid19, or with Adult Respiratory Distress Syndrome (ARDS). CS has been attributed as a major cause of morbidity, multi-organ failure and mortality in patients having a number of diseases, for example, inflammatory diseases, autoimmune diseases, cancer and infectious diseases, including viral infection, e.g., SARS-Covid19, or Adult Respiratory Distress Syndrome (ARDS). In CS, the uncontrolled increase in IL-6, as well as other pro-inflammatory cytokines, results in an influx of immune cells leading to progressive tissue destruction. The anti-hIL-6 VHH antibodies described herein may provide therapeutic intervention to blunt, diminish, neutralize, or block hIL-6-driven CS. Moreover, the described anti-hIL-6 VHH antibodies provide new therapeutics and treatments for other IL-6-mediated pathologies, such as, without limitation, autoimmune diseases, inflammatory diseases, and cancers, such as subsets of cholangiocarcinomas and hepatocellular adenomas.

### Example 7—Monomeric and Dimeric Forms of Anti-Human IL-6 VHH-Binding Molecules

Presented below are the amino acid (polypeptide) sequences of a representative anti-hIL-6 VHH antibody monomer, JYK-D12, and a homodimer of JYK-D12, namely, the JYK-D12 homodimer, as described herein. The two JYK-D12 monomers that comprise the homodimer are separated by a flexible spacer or linker, which is underlined. The JYK-D12/JYK-D12 homodimer (“JYK-D12 homodimer”), which shows highly potent and specific binding to hIL-6, comprises the amino acid sequence, in an NH₂ to —COOH orientation, as follows:

(SEQ ID NO: 87)
QVQLVESGGGLVQPGGSLGLSCAASGFTLAYYGIGWFRQAPGKEREGVACISSSDLSTYYADSV
KGRFTISRDNAKDTVYLQMNSLKPEDTAVYYCAAGTWDLKEGYSRSNCVRSYEYDYWGQGTQVT
VSSGGGGSGGGGSGGGGSQVQLVESGGGLVQPGGSLGLSCAASGFTLAYYGIGWFRQAPGKERE
GVACISSSDLSTYYADSVKGRFTISRDNAKDTVYLQMNSLKPEDTAVYYCAAGTWDLKEGYSRS
NCVRSYEYDYWGQGTQVTVSS

or an amino acid sequence having at least 85%, at least 90%, at least 95%, at least 98%, or at least 99% amino acid sequence identity to the JYK-D12 homodimer sequence.

The amino acid sequence of the JYK-D12 VHH antibody monomeric form is as follows:

(SEQ ID NO: 88)
QVQLVESGGGLVQPGGSLGLSCAASGFTLAYYGIGWFRQAPGKEREGVACISSSDLSTYYADSV
KGRFTISRDNAKDTVYLQMNSLKPEDTAVYYCAAGTWDLKEGYSRSNCVRSYEYDYWGQGTQVT
VSS

The JYK-D12 homodimer was recombinantly produced and expressed with a leader sequence as follows:

(SEQ ID NO: 89)
MGWSCIILFLVATATGVHSQVQLVESGGGLVQPGGSLGLSCAASGFTLAYYGIGWFRQAPGKER
EGVACISSSDLSTYYADSVKGRFTISRDNAKDTVYLQMNSLKPEDTAVYYCAAGTWDLKEGYSR
SNCVRSYEYDYWGQGTQVTVSSGGGGSGGGGSGGGGSQVQLVESGGGLVQPGGSLGLSCAASGF
TLAYYGIGWFRQAPGKEREGVACISSSDLSTYYADSVKGRFTISRDNAKDTVYLQMNSLKPEDT
AVYYCAAGTWDLKEGYSRSNCVRSYEYDYWGQGTQVTVSS

The leader sequence at the NH₂-terminus of the recombinantly expressed homodimer is designated in the above sequence in bold, italicized font.

In a particular, nonlimiting embodiment, an amino acid sequence suitable for JYK-D12 homodimer expression was produced as follows:

(SEQ ID NO: 90)
MGWSCIILFLVATATGVHSQVQLVESGGGLVQPGGSLGLSCAASGFTLAYYGIGWFRQAPGKER
EGVACISSSDLSTYYADSVKGRFTISRDNAKDTVYLQMNSLKPEDTAVYYCAAGTWDLKFGYSR
SNCVRSYEYDYWGQGTQVTVSSGGGGSGGGGSGGGGSQVQLVESGGGLVQPGGSLGLSCAASGF
TLAYYGIGWFRQAPGKEREGVACISSSDLSTYYADSVKGRFTISRDNAKDTVYLQMNSLKPEDT

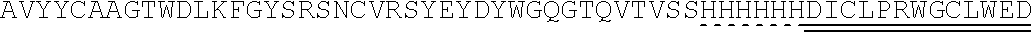

In the above homodimer sequence, the leader sequence at the NH₂-terminus of the homodimer is designated in bold, italicized font; a flexible spacer sequence is designated by single underlining; a hexa-histidine (H) tag (SEQ ID NO: 53) is designated by dotted underlining; and an albumin binding domain is designated by double underlining. As will be appreciated by the skilled practitioner, the leader sequence is an optional component of the homodimer and is typically included for expression and secretion of a recombinant protein from a cell; the histidine tag is an optional component of the homodimer and is included for facilitating purification of the polypeptide; and the albumin binding domain (APB) component of the homodimer is optional and may be included to prolong the pharmacokinetic properties of the polypeptide, e.g., in in vivo and/or preclinical studies. In some embodiments, a recombinant anti-hIL-6 VHH polypeptide monomer or dimer, e.g., homodimer, may include one or more epitope tags (E-tags) as described herein. In an embodiment, such an E-tag may be included at the carboxy (—COOH) terminus of the polypeptide.

By way of example, FIG. 8 presents the amino acid sequence and the encoding nucleic acid sequence of the JYK-D12 homodimer, which was recombinantly expressed in Expi293F cells using the mammalian expression plasmid vector pcDN3.4. Also shown in FIG. 8 is a linear depiction of the expression plasmid encoding the JYK-D12 anti-hIL-6 VHH antibody homodimer. The expression plasmid includes the following components, from left to right: EcoR1 restriction enzyme site; Kozak sequence; artificial signal peptide; dimer of JYK-D12 anti-hIL-6 VHH antibody; histidine tag (his-tag); stop codon; and HindIII restriction enzyme site.

Polynucleotides encoding the monomer and dimer (and other multimer) forms of the anti-hIL-6 VHH molecules described herein may be encoded by a nucleic acid or a nucleic acid construct. In embodiments, the nucleic acid encoding the anti-hIL-6 VHH monomer, dimer, or multimer is DNA or RNA. In an embodiment, the nucleic acid encoding the anti-hIL-6 VHH monomer, dimer, or multimer is mRNA.

### Example 8—Materials and Methods

### Enzyme Linked Immunosorbent Assay (ELISA)

Maxisorp ELISA plates (Nunc; Thermo Fisher USA) coated with recombinantly produced hIL-6 protein (0.5-5 μg/ml) overnight at 4° C. were used for immuno-binding assays (ELISA). Plates were washed 3 times with 1×PBS+0.1% Tween, followed by washing 3 times with 1×PBS. Washed plates were blocked (4-5% non-fat dry milk in 1×PBS+0.1% Tween) for 1 hour at room temperature (RT) with rocking. Serially diluted (1:5) hIL-6 VHH binding molecules targeting hIL-6, diluted in blocking solution, were incubated for 1 hour at RT with rocking and washed as above. Equivalent control samples were spiked with a known amount of an irrelevant VHH for use as an internal standard.

Binding of the VHHs to recombinant hIL-6 protein coating the wells was detected at A450 nm using horse radish peroxidase (HRP)-labeled anti-E-tag antibody and an ELISA reader. Bound HRP was detected using 3,3′,5,5′-tetramethylbenzidine (TMB substrate, Sigma) and values were plotted as a function of the input VHH concentration. Illustratively, the plates were incubated with goat anti-E-tag-HRP conjugated antibody (Bethyl labs) diluted 1:5000 in blocking solution for 1 hour at RT with rocking and were washed as above before adding TMB microwell peroxidase substrate (KPL) to develop (incubated for 10-40 min). Development was stopped with 1M H₂SO₄ and the plates were read at 450 nm on an ELx808 Ultra Microplate Reader (Bio-Tek instruments), (Mukherjee, J. et al., 2012, PloS ONE 7:e29941). VHH levels in unknown samples were determined by comparison of their signals to those of internal standards as previously described (Mukherjee, J. et al., 2014, PLoS One 9:e106422; Sheoran, A S et al., 2015, Infect Immun, 83:286-291; Moayeri, M. et al., 2016, Clin Vaccine Immunol, doi:10.1128/cvi.00611-15; Sponseller, J K et al., 2014, J Infect Dis, doi:10.1093/infdis/jiu605; Tzipori, S. et al., 1995, Infect Immun, 63:3621-3627). EC₅₀ values were calculated for the VHH concentration that secreted in a signal equal to 50% of the maximum signal.

### Computational Analysis

In general, data were analyzed using GraphPad Prism software version 6. All error bars refer to standard deviations. ELISA data were analyzed using nonlinear regression.

### Animal Experiments

Experiments using alpacas (camelids) involving the production of anti-hIL-6 VHHs as described herein were conducted using animals housed under standard and humane conditions with a standard commercial alpaca diet and tap water provided to the animals ad libitum. All experiments involving animals were performed under protocols approved by Tufts University and National Institute of Allergy and Infectious Diseases (NIAID) Animal Care and Use Committees. Work with alpacas was performed at Tufts under approved protocol Tuskegee University School of Veterinary Medicine (TUSVM) and Institutional Animal Care and Use Committee (IACUC) Protocol #G2015-49.

All publications, patents, published patent applications and sequence database entries mentioned and disclosed herein are hereby incorporated by reference in their entireties as if each individual publication or patent were specifically and individually indicated to be incorporated by reference.

What is claimed is:

1. A VH-heavy chain only (VHH) binding protein or an antigen binding portion thereof that specifically binds to interleukin-6 (IL-6), wherein the binding protein or the antigen binding portion thereof comprises three Complementarity Determining Regions (CDRs), CDR1, CDR2 and CDR3, which are structurally positioned between four camelid VHH framework (FR) regions (FR1-FR4) as follows: FR1-CDR1-FR2-CDR2-FR3-CDR3-FR4; wherein the three CDRs are selected from:
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13);
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13);
CDR1 comprising amino acid sequence GFTLDYYA (SEQ ID NO: 11); CDR2 comprising amino acid sequence SSSDRSAY (SEQ ID NO: 14); and CDR3 comprising amino acid sequence GTWDLKWGYNISACVRSYEYDY (SEQ ID NO: 15);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFALDYYA (SEQ ID NO: 18); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GFTLDYYG (SEQ ID NO: 19); CDR2 comprising amino acid sequence SSSDLKTY (SEQ ID NO: 16); and CDR3 comprising amino acid sequence GTWDLKFGYNITTCVRSSEYDY (SEQ ID NO: 20);
CDR1 comprising amino acid sequence GFTSDYYG (SEQ ID NO: 21); CDR2 comprising amino acid sequence SSSDLKTY (SEQ ID NO: 16); and CDR3 comprising amino acid sequence GTWDLKFGYNITTCVRSSEYDY (SEQ ID NO: 20);
CDR1 comprising amino acid sequence GFTLDYYG (SEQ ID NO: 19); CDR2 comprising amino acid sequence SSSDWSTY (SEQ ID NO: 22); and CDR3 comprising amino acid sequence GTWDLKFGYNRSNCVRSAEYDY (SEQ ID NO: 23); or
CDR1 comprising amino acid sequence GFTLAYYG (SEQ ID NO: 24); CDR2 comprising amino acid sequence SSSDLSTY (SEQ ID NO: 25); and CDR3 comprising amino acid sequence GTWDLKFGYSRSNCVRSYEYDY (SEQ ID NO: 26).

2. The VHH binding protein of claim 1, wherein
FR1 comprises 20 consecutive amino acids comprising X₁-X₂-G-G-G-L-V-Q-P-G-G-S-X₃-X₄-L-S-C-A-A-S(SEQ ID NO: 27), wherein X₁ is absent or T; X₂ is S, T, or G; X₃ is L or Q; and X₄ is R or G;
FR2 comprises 18 consecutive amino acids comprising X₁-G-W-F-R-Q-A-P-G-K-E-R-E-G-X₂-X₃-C-X₄ (SEQ ID NO: 28), wherein X₁ is I or V; X₂ is V or I; X₃ is S or A; and X₄ is L, I, or M;
FR3 comprises 38 consecutive amino acids comprising X₁-D-S-V-K-G-R-F-T-I-S-R-D-X₂-X₃-X₄-X₅-X₆-X₇-X₈-L-Q-M-N-S-L-K-P-E-D-T-X₉-X₁₀-Y-Y-C-A-A (SEQ ID NO: 29), wherein X₁ is V, I, T, or A; X₂ is D, G, N, Y, or S; X₃ is D or A; X₄ is K or N; X₅ is N, S, or D; X₆ is T or A; X₇ is A or V; X₈ is Y or S; X₉ is A or G; and X₁₀ is T or V; and
FR4 comprises 11 consecutive amino acids comprising X₁-X₂-Q-G T Q V T V S S (SEQ ID NO: 30), wherein X₁ is W or R; and X₂ is G or D.

3. A VH-heavy chain only (VHH) binding protein or an antigen binding portion thereof that specifically binds to interleukin-6 (IL-6), wherein the binding protein or the antigen binding portion thereof comprises three Complementarity Determining Regions (CDRs), CDR1, CDR2 and CDR3, which are structurally positioned between four camelid VHH framework (FR) regions (FR1-FR4) as follows: FR1-CDR1-FR2-CDR2-FR3-CDR3-FR4; wherein the three CDRs are selected from:
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GRPFSSFA (SEQ ID NO: 31); CDR2 comprising amino acid sequence TWSRGTTH (SEQ ID NO: 32); and CDR3 comprising amino acid sequence AAADGWKVVSTASPAYDY (SEQ ID NO: 33);
CDR1 comprising amino acid sequence GFTLAYYG (SEQ ID NO: 24); CDR2 comprising amino acid sequence SSSDLSTY (SEQ ID NO: 25); and CDR3 comprising amino acid sequence GTWDLKFGYSRSNCVRSYEYDY (SEQ ID NO: 26);
CDR1 comprising amino acid sequence GRTFSSRA (SEQ ID NO: 34); CDR2 comprising amino acid sequence SWTGSPY (SEQ ID NO: 35); and CDR3 comprising amino acid sequence AATSEHVMLVVTTRGGYDY (SEQ ID NO: 36); or
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13).

4. The VHH binding protein of claim 3, wherein
FR1 comprises 25 consecutive amino acids comprising Q-X₁-Q-L-X₂-E-X₃-G-G-G-X₄-V-Q-X₅-G-X₆-S-L-X₇-L-S-C-A-A-S(SEQ ID NO: 37), wherein X₁ is L or V; X₂ is A or V; X₃ is T or S; X₄ is L or S; X₅ is P or A; X₆ is G or D; X₇ is R, T or G;
FR2 comprises 18 consecutive amino acids comprising X₁-G-W-F-R-Q-A-P-G-K-E-R-E-X₂-X₃-X₄-X₅-X₆ (SEQ ID NO: 38), wherein X₁ is V, M, or I; X₂ is F or G; X₃ is I or V; X₄ is S or A; X₅ is C, A or V; X₆ is I or L;
FR3 comprises 37-39 consecutive amino acids comprising Y-X₁-D-S-V-K-G-R-F-T-I-S-X₂-D-X₃-X₄-K-X₅-T-X₆-X₇-L-Q-M-N-S-L-K-P-E-D-T-X₈-X₉-Y-Y-C-A-A (SEQ ID NO: 39), wherein X₁ is A, T, or V; X₂ is R or G; X₃ is Y, N, or D; X₄ is A or D; X₅ is S, N, or D; X₆ is V or A; X₇ is S, F, or Y; X₈ is G or A; X₉ is V or T; and
FR4 comprises 11 consecutive amino acids comprising W-X₁-Q-G-T-Q-V-T-V-S-S (SEQ ID NO: 40), wherein X₁ is D or G.

5. The VHH binding protein of claim 1, wherein the protein neutralizes hIL-6 activity in vitro or in vivo; or wherein the protein reduces or abolishes JAK-STAT signaling in vitro or in vivo.

6. The VHH binding protein of claim 1, which is recombinantly produced.

7. The VHH binding protein of claim 1, which is in the form of a dimer, a homodimer, or a multimer.

8. The VHH binding protein of claim 1, comprising one or more epitope tag sequences specifically bindable by an anti-epitope tag antibody or a binding portion thereof, optionally, wherein the one or more epitope tag sequences comprises at least one of DELGPRLMGK (SEQ ID NO: 41) or GAPVPYPDPLEPR (SEQ ID NO: 42).

9. A polypeptide that specifically binds to human interleukin-6 (hIL-6), wherein the polypeptide or a hIL-6-binding portion thereof has at least 85% amino acid sequence identity to a sequence selected from the group consisting of
(SEQ ID NO: 1)
QLQLAETGGGLVQPGGSLRLSCAASGFTLDYYAVGWFRQAPGKEREGIS
CISSSDLKTYYADSVKGRFTISRDYAKSTVSLQMNSLKPEDTGVYYCAA
GTWDLKEGYNISACVGSYEYDYWDQGTQVTVSS;
(SEQ ID NO: 3)
QVQLVESGGGLVQAGDSLTLSCAASGRPFSSFAMGWFRQAPGKEREFVA
AITWSRGTTHYADSVKGRFTISGDNAKNTVFLQMNSLKPEDTAVYYCAA
ADGWKVVSTASPAYDYWGQGTQVTVSS;
(SEQ ID NO: 5)
QLQLVESGGGLVQPGGSLGLSCAASGFTLAYYGIGWFRQAPGKEREGVA
CISSSDLSTYYADSVKGRFTISRDNAKDTVYLQMNSLKPEDTAVYYCAA
GTWDLKFGYSRSNCVRSYEYDYWGQGTQVTVSS;
(SEQ ID NO: 7)
QVQLAETGGGSVQAGGSLTLSCAASGRTFSSRAMGWFRQAPGKEREFVA
VISWTGSPYYTDSVKGRFTISRDDAKNTVYLQMNSLKPEDTAVYYCAAT
SEHVMLVVTTRGGYDYWGQGTQVTVSS;
and
(SEQ ID NO: 9)
QLQLVETGGGLVQPGGSLRLSCAASGFTLDYYAIGWFRQAPGKEREGVS
CLSSSDRSTYYVDSVKGRFTISRDDDKNTAYLQMNSLKPEDTATYYCAA
GTWDLKWGYNISACVGSYEYDYWGQGTQVTVSS.

10. The polypeptide of claim 9, wherein conservative amino acid substitutions in the polypeptide comprise the at least 85% amino acid sequence identity.

11. A polypeptide that specifically binds to human interleukin-6 (hIL-6), wherein the polypeptide or a hIL-6-binding portion thereof comprises or consists of a sequence selected from the group consisting of SEQ ID NOs: 1, 3, 5, 7 and 9.

12. The polypeptide of claim 9, wherein the polypeptide neutralizes hTL-6 activity in vitro or in vivo, or wherein the polypeptide reduces or abolishes JAK-STAT signaling in vitro or in vivo.

13. The polypeptide of claim 9, which is in the form of a dimer, a homodimer, or a multimer.

14. The polypeptide of claim 9, comprising one or more epitope tag sequences specifically bindable by an anti-epitope tag antibody or binding portion thereof.

15. A dimeric or multimeric polypeptide comprising two or more anti-hIL-6 VHH polypeptides comprising a sequence selected from the group consisting of SEQ ID NOs: 1, 3, 5, 7, and 9, or hIL-6-binding regions thereof, wherein the two or more anti-hTL-6 VHH polypeptides, or hIL-6-binding regions, are joined with one or more spacer or linker peptides.

16. The dimeric or multimeric polypeptide of claim 15, wherein the polypeptide comprises one or more epitope tag sequences specifically bindable by an anti-epitope tag antibody or binding portion thereof, and/or wherein the polypeptide is linked to an immunoglobulin Fc domain.

17. The dimeric or multimeric polypeptide of claim 15, wherein the polypeptide is dimeric and comprises two anti-hIL-6 VHH polypeptides.

18. The dimeric polypeptide of claim 17, wherein the two anti-hIL-6 VHH polypeptides are the same and are in the form of a homodimer or wherein the two anti-hTL-6 VHH polypeptides are different and are in the form of a heterodimer.

19. The dimeric or multimeric polypeptide of claim 15, wherein the polypeptide is multimeric and comprises at least three or at least four same or different anti-hIL-6 VHH polypeptides.

20. The binding protein of claim 1, which is linked to an immunoglobulin Fc domain.

21. An isolated polynucleotide encoding the dimeric or multimeric polypeptide of claim 15.

22. An isolated polynucleotide having at least 90% sequence identity to a nucleic acid sequence selected from the group consisting of
(SEQ ID NO: 2)
cagttgcagctggcggagactggtggagggttggtccagcctggggggtctctgagactctcct
gtgcagcctctggattcactttggattattatgccgtaggctggttccgccaggccccagggaa
ggagcgtgaggggatctcatgtattagtagtagtgatcttaaaacatactatgcagactccgtg
aagggccgattcaccatctccagagactacgccaagagcacggtgtctctgcaaatgaacagcc
tgaaacctgaggacacaggcgtttattactgtgcggcgggcacatgggatcttaagttcggcta
taatattagtgcctgcgtgggatcttatgagtatgactactgggaccaggggacccaggtcacc
gtctcctca;
(SEQ ID NO: 4)
caggtgcagctcgtggagtcaggaggaggattggtgcaggctggggactctctgacactctcct
gtgcagcctctggacgccccttcagtagttttgccatgggctggttccgccaggctccagggaa
ggagcgtgagtttgtagcagctattacatggagtcgtggtaccacacactatgccgactccgtg
aagggccggttcaccatctccggggacaacgccaagaacacggtgtttctgcaaatgaacagcc
taaaacctgaggatacggccgtttattactgtgcagcagcggatggatggaaggtagttagtac
tgctagccccgcgtatgactactggggccaggggacccaggtcaccgtctcctca;
(SEQ ID NO: 6)
cagttgcagctggtggagtccggtggaggcttggtgcagcctggggggtctctgggactctcct
gtgcagcctctggattcactttggcttattatggcataggctggttccgccaggccccagggaa
ggagcgtgagggggtcgcatgtattagtagtagtgatcttagcacatactatgcagactccgtg
aagggccgattcaccatctccagagacaacgccaaggacacggtgtatctgcaaatgaacagcc
tgaaacctgaggacacagccgtttattactgtgcagcgggcacatgggatcttaaattcggcta
tagtagaagtaactgcgtgcgatcttatgagtatgactactggggccaggggacccaggtcacc
gtctcctca;
(SEQ ID NO: 8)
caggtgcagctggcggagaccggcggaggatcggtgcaggctgggggctctctgacactctcct
gtgcagcctctggacgcaccttcagtagcagagccatgggctggttccgccaggctccagggaa
ggagcgtgagtttgtagcagttattagctggactggtagcccatactatacagactccgtgaag
ggccgattcaccatctccagagacgacgccaagaacacggtgtatctgcaaatgaacagcctga
aacctgaggacacggccgtttattactgcgcagcgacgtcagaacatgtaatgctggtagttac
tacgcgtggcgggtatgactactggggccaggggacccaggtcaccgtctcctca;
and
(SEQ ID NO: 10)
cagttgcagctggtggagacaggaggaggcttggtgcagcctggggggtctctgagactctcct
gtgcagcctctggattcactttggattattatgccataggctggttccgccaggctccagggaa
ggagcgtgagggggtctcatgtttgagtagtagtgatcgtagcacatactatgtagactccgtg
aagggccgattcaccatctccagagacgatgacaagaacacggcgtatctgcagatgaacagcc
tgaaacctgaggacacagccacttattactgtgcagcgggcacatgggatcttaaatggggcta
taacattagtgcctgcgtgggatcttatgagtatgactactggggccaggggacgcaggtcacc
gtctcctca.

23. An isolated polynucleotide comprising or consisting of a sequence selected from the group consisting of SEQ ID NOs: 2, 4, 6, 8 and 10.

24. An isolated polynucleotide comprising a nucleic acid sequence encoding an anti-hIL-6 VHH of any one of SEQ ID NOs: 1, 3, 5, 7, or 9.

25. A vector or expression vector comprising a nucleic acid molecule that encodes the binding protein of claim 1.

26. A vector or expression vector comprising a nucleic acid molecule that encodes the dimeric or multimeric polypeptide of claim 15.

27. A host cell comprising the vector or expression vector of claim 25.

28. A pharmaceutical composition comprising an effective amount of the binding protein of claim 1, or a hIL-6 binding fragment thereof, or an isolated polynucleotide encoding the binding protein or the binding fragment thereof, and a pharmaceutically acceptable excipient, carrier, or diluent.

29. A pharmaceutical composition comprising an effective amount of the dimeric or multimeric polypeptide of claim 15, or a hIL-6 binding fragment thereof, or an isolated polynucleotide encoding the dimeric or multimeric polypeptide, and a pharmaceutically acceptable excipient, carrier, or diluent.

30. A method of neutralizing interleukin-6 (IL-6) activity and/or inhibiting interleukin-6 (IL-6)-induced STAT3 activation, the method comprising contacting a cell with an effective amount of the binding protein of claim 1, or an isolated polynucleotide encoding the binding protein, thereby neutralizing IL-6 activity and/or inhibiting IL-6-induced STAT3 activation.

31. The method of claim 30, wherein the cell is in vitro, ex vivo, or in vivo and/or wherein the cell is an hepatocyte, an endothelial cell, a monocyte, a macrophage, a T cell, a B cell, a fibroblast, a keratinocyte, or an adipocyte.

32. A method of treating an interleukin-6 (IL-6)-mediated disease, disorder, pathology or infection and/or the symptoms thereof in a subject, the method comprising administering to a subject in need thereof an effective amount of the pharmaceutical composition of claim 28, thereby treating the IL-6-mediated disease, disorder, pathology or infection and/or the symptoms thereof in the subject.

33. A method of ameliorating, abrogating, or treating cytokine storm associated with an interleukin-6 (IL-6)-mediated disease, disorder, pathology or infection and/or the symptoms thereof in a subject, the method comprising administering to a subject in need thereof an effective amount of the pharmaceutical composition of claim 28, thereby ameliorating, abating, or treating cytokine storm and/or the symptoms thereof in the subject.

34. The method of claim 32, wherein the IL-6-mediated disease, disorder, pathology or infection is a viral infection or a bacterial infection, a cancer, a carcinoma, a tumor, a cholangiocarcinoma, ovarian cancer, multiple myeloma; an autoimmune disease, an inflammatory disease, adult rheumatoid arthritis, juvenile idiopathic arthritis, Castleman's disease, secondary amyloidosis, polymyalgia rheumatic, adult onset Still's disease, polymyositis, systemic sclerosis, large vessel vasculitis lupus erythematosus, Crohn's disease, irritable bowel disease (IBD), Sjogren's syndrome; steroid refractory Graft versus Host Disease in transplantation; type 2 diabetes, obesity, or schizophrenia.

35. The method of claim 34, wherein the IL-6 mediated disease, disorder, or infection is a viral infection, which is a Covid-19 infection or Adult Respiratory Distress Syndrome (ARDS).

36. A polypeptide that specifically binds to and neutralizes human interleukin-6 (hIL-6), or a hIL-6-binding portion thereof, wherein the polypeptide comprises three complementarity determining regions (CDRs), CDR1, CDR2 and CDR3, and four camelid VHH framework regions (FRs), FR1, FR2, FR3 and FR4, wherein the FRs structurally and positionally support CDR1-CDR3 therebetween as follows: FR1-CDR1-FR2-CDR2-FR3-CDR3-FR4, and wherein the three CDRs are selected from:
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GRPFSSFA (SEQ ID NO: 31); CDR2 comprising amino acid sequence TWSRGTTH (SEQ ID NO: 32); and CDR3 comprising amino acid sequence AAADGWKVVSTASPAYDY (SEQ ID NO: 33);
CDR1 comprising amino acid sequence GFTLAYYG (SEQ ID NO: 24); CDR2 comprising amino acid sequence SSSDLSTY (SEQ ID NO: 25); and CDR3 comprising amino acid sequence GTWDLKFGYSRSNCVRSYEYDY (SEQ ID NO: 26);
CDR1 comprising amino acid sequence GRTFSSRA (SEQ ID NO: 34); CDR2 comprising amino acid sequence SWTGSPY (SEQ ID NO: 35); and CDR3 comprising amino acid sequence AATSEHVMLVVTTRGGYDY (SEQ ID NO: 36); or
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13); and wherein the four VHH FRs are camelid anti-hIL-6 VHH FRs.

37. The polypeptide of claim 36, the four camelid anti-hIL-6 VHH FRs comprise the following:
FR1 comprises 25 consecutive amino acids comprising Q-X₁-Q-L-X₂-E-X₃-G-G-G-X₄-V-Q-X₅-G-X₆-S-L-X₇-L-S-C-A-A-S(SEQ ID NO: 37), wherein X₁ is L or V; X₂ is A or V; X₃ is T or S; X₄ is L or S; X₅ is P or A; X₆ is G or D; X₇ is R, T or G;
FR2 comprises 18 consecutive amino acids comprising X₁-G-W-F-R-Q-A-P-G-K-E-R-E-X₂-X₃-X₄-X₅-X₆ (SEQ ID NO: 38), wherein X₁ is V, M, or I; X₂ is F or G; X₃ is I or V; X₄ is S or A; X₅ is C, A or V; X₆ is I or L;
FR3 comprises 37-39 consecutive amino acids comprising Y-X₁-D-S-V-K-G-R-F-T-I-S-X₂-D-X₃-X₄-K-X₅-T-X₆-X₇-L-Q-M-N-S-L-K-P-E-D-T-X₈-X₉-Y-Y-C-A-A (SEQ ID NO: 39), wherein X₁ is A, T, or V; X₂ is R or G; X₃ is Y, N, or D; X₄ is A or D; X₅ is S, N, or D; X₆ is V or A; X₇ is S, F, or Y; X₈ is G or A; X₉ is V or T; and
FR4 comprises 11 consecutive amino acids comprising W-X₁-Q-G-T-Q-V-T-V-S-S(SEQ ID NO: 40), wherein X₁ is D or G.

38. The polypeptide of claim 36, which is in the form of a dimer, a homodimer, or a multimer.

39. The polypeptide of claim 36, wherein the polypeptide comprises one or more epitope tag sequences specifically bindable by an anti-epitope tag antibody or binding portion thereof, and/or wherein the polypeptide is linked to an immunoglobulin Fc domain.

40. A pharmaceutical composition comprising an effective amount of the polypeptide of claim 36 or a hIL-6-binding fragment thereof, or an isolated polynucleotide encoding the polypeptide or the binding fragment thereof, and a pharmaceutically acceptable excipient, carrier, or diluent.

41. A method of inhibiting or abrogating interleukin-6 (IL-6)-induced STAT3 activation in a subject, the method comprising administering to a subject in need thereof an effective amount of the pharmaceutical composition of claim 40, thereby, thereby inhibiting IL-6-induced STAT3 activation in the subject.

42. A kit comprising the binding protein of claim 1, an isolated polynucleotide encoding the binding protein, or a pharmaceutical composition thereof, for treating or protecting against an interleukin-6 (IL-6)-mediated disease, disorder, condition, pathology, or infection and/or the symptoms thereof, and optionally comprising instructions for use.

43. A method of detecting interleukin 6 (IL-6) or a peptide thereof in a sample, the method comprising:
contacting the sample with at least one detectably labeled VHH binding protein or an antigen binding fragment thereof that specifically binds to IL-6 or a peptide thereof, wherein the binding protein or the antigen binding fragment thereof comprises three Complementarity Determining Regions (CDRs), CDR1, CDR2 and CDR3 structurally positioned between four framework (FR) regions (FR1-FR4) as follows: FR1-CDR1-FR2-CDR2-FR3-CDR3-FR4; wherein the three CDRs are selected from:
CDR1 comprising amino acid sequence GFTLDYYA (SEQ ID NO: 11); CDR2 comprising amino acid sequence SSSDLKTY (SEQ ID NO: 16); and CDR3 comprising amino acid sequence GTWDLKFGYNISACVGSYEYDY (SEQ ID NO: 17);
CDR1 comprising amino acid sequence GRPFSSFA (SEQ ID NO: 31); CDR2 comprising amino acid sequence TWSRGTTH (SEQ ID NO: 32); and CDR3 comprising amino acid sequence AAADGWKVVSTASPAYDY (SEQ ID NO: 33);
CDR1 comprising amino acid sequence GFTLAYYG (SEQ ID NO: 24); CDR2 comprising amino acid sequence SSSDLSTY (SEQ ID NO: 25); and CDR3 comprising amino acid sequence GTWDLKFGYSRSNCVRSYEYDY (SEQ ID NO: 26);
CDR1 comprising amino acid sequence GRTFSSRA (SEQ ID NO: 34); CDR2 comprising amino acid sequence SWTGSPY (SEQ ID NO: 35); and CDR3 comprising amino acid sequence AATSEHVMLVVTTRGGYDY (SEQ ID NO: 36); or
CDR1 comprising amino acid sequence GFTLDYYA (SEQ ID NO: 11); CDR2 comprising amino acid sequence SSSDRSTY (SEQ ID NO: 12); and CDR3 comprising amino acid sequence GTWDLKWGYNISACVGSYEYDY (SEQ ID NO: 13); and wherein
FR1 comprises 25 consecutive amino acids comprising Q-X₁-Q-L-X₂-E-X₃-G-G-G-X₄-V-Q-X₅-G-X₆-S-L-X₇-L-S-C-A-A-S(SEQ ID NO: 37), wherein X₁ is L or V; X₂ is A or V; X₃ is T or S; X₄ is L or S; X₅ is P or A; X₆ is G or D; X₇ is R, T or G;
FR2 comprises 18 consecutive amino acids comprising X₁-G-W-F-R-Q-A-P-G-K-E-R-E-X₂-X₃-X₄-X₅-X₆ (SEQ ID NO: 38), wherein X₁ is V, M, or I; X₂ is F or G; X₃ is I or V; X₄ is S or A; X₅ is C, A or V; X₆ is I or L;
FR3 comprises 37-39 consecutive amino acids comprising Y-X₁-D-S-V-K-G-R-F-T-I-S-X₂-D-X₃-X₄-K-X₅-T-X₆-X₇-L-Q-M-N-S-L-K-P-E-D-T-X₈-X₉-Y-Y-C-A-A (SEQ ID NO: 39), wherein X₁ is A, T, or V; X₂ is R or G; X₃ is Y, N, or D; X₄ is A or D; X₅ is S, N, or D; X₆ is V or A; X₇ is S, F, or Y; X₈ is G or A; X₉ is V or T; and
FR4 comprises 11 consecutive amino acids comprising W-X₁-Q-G-T-Q-V-T-V-S-S (SEQ ID NO: 40), wherein X₁ is D or G; under conditions for the binding protein to interact with IL-6; and
measuring the level of binding of the binding protein to IL-6 in the sample relative to a control to detect or identify the presence of IL-6 in the sample.

44. The method of claim 24, wherein the isolated polynucleotide comprises mRNA, which is formulated with a delivery agent.

45. The method of claim 44, wherein the delivery agent comprises one or more of nanoparticles, lipid nanoparticles, ionizable lipids; biodegradable ionizable lipids; polymeric materials, polyethyleneimines (PEIs), poly(glycoamidoamine) polymers, poly(glycoamidoamine) polymers modified with fatty chains, poly(β-amino)esters (PBAEs), polymethacrylates; dendrimers, polyamidoamine (PAMAM), polypropylenimine-based dendrimers, PAMAM (generation 0) dendrimer co-formulated with poly(lactic-co-glycolic acid) (PLGA) and ceramide-PEG; cell penetrating peptides; cationic lipids; or zwitterionic lipids.


SEQUENCE LISTING (105 sequences)

SEQ ID NO: 1 - protein, 131 aa, synthetic construct
  REGION 1..131: note = Description of Artificial Sequence: Synthetic polypeptide
QLQLAETGGGLVQPGGSLRLSCAASGFTLDYYAVGWFRQAPGKEREGISCISSSDLKTYYADSVKGRFTISRDYAKSTVSLQMNSLKPEDTGVYYCAAGTWDLKFGYNISACVGSYEYDYWDQGTQVTVSS

SEQ ID NO: 2 - DNA, 393 bp, synthetic construct
  misc_feature 1..393: note = Description of Artificial Sequence: Synthetic polynucleotide
  source 1..393: mol_type = other DNA
cagttgcagctggcggagactggtggagggttggtccagcctggggggtctctgagactctcctgtgcagcctctggattcactttggattattatgccgtaggctggttccgccaggccccagggaaggagcgtgaggggatctcatgtattagtagtagtgatcttaaaacatactatgcagactccgtgaagggccgattcaccatctccagagactacgccaagagcacggtgtctctgcaaatgaacagcctgaaacctgaggacacaggcgtttattactgtgcggcgggcacatgggatcttaagttcggctataatattagtgcctgcgtgggatcttatgagtatgactactgggaccaggggacccaggtcaccgtctcctca

SEQ ID NO: 3 - protein, 125 aa, synthetic construct
  REGION 1..125: note = Description of Artificial Sequence: Synthetic polypeptide
QVQLVESGGGLVQAGDSLTLSCAASGRPFSSFAMGWFRQAPGKEREFVAAITWSRGTTHYADSVKGRFTISGDNAKNTVFLQMNSLKPEDTAVYYCAAADGWKVVSTASPAYDYWGQGTQVTVSS

SEQ ID NO: 4 - DNA, 375 bp, synthetic construct
  misc_feature 1..375: note = Description of Artificial Sequence: Synthetic polynucleotide
  source 1..375: mol_type = other DNA
caggtgcagctcgtggagtcaggaggaggattggtgcaggctggggactctctgacactctcctgtgcagcctctggacgccccttcagtagttttgccatgggctggttccgccaggctccagggaaggagcgtgagtttgtagcagctattacatggagtcgtggtaccacacactatgccgactccgtgaagggccggttcaccatctccggggacaacgccaagaacacggtgtttctgcaaatgaacagcctaaaacctgaggatacggccgtttattactgtgcagcagcggatggatggaaggtagttagtactgctagccccgcgtatgactactggggccaggggacccaggtcaccgtctcctca

SEQ ID NO: 5 - protein, 131 aa, synthetic construct
  REGION 1..131: note = Description of Artificial Sequence: Synthetic polypeptide
QLQLVESGGGLVQPGGSLGLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVSS

SEQ ID NO: 6 - DNA, 393 bp, synthetic construct
  misc_feature 1..393: note = Description of Artificial Sequence: Synthetic polynucleotide
  source 1..393: mol_type = other DNA
cagttgcagctggtggagtccggtggaggcttggtgcagcctggggggtctctgggactctcctgtgcagcctctggattcactttggcttattatggcataggctggttccgccaggccccagggaaggagcgtgagggggtcgcatgtattagtagtagtgatcttagcacatactatgcagactccgtgaagggccgattcaccatctccagagacaacgccaaggacacggtgtatctgcaaatgaacagcctgaaacctgaggacacagccgtttattactgtgcagcgggcacatgggatcttaaattcggctatagtagaagtaactgcgtgcgatcttatgagtatgactactggggccaggggacccaggtcaccgtctcctca

SEQ ID NO: 7 - protein, 125 aa, synthetic construct
  REGION 1..125: note = Description of Artificial Sequence: Synthetic polypeptide
QVQLAETGGGSVQAGGSLTLSCAASGRTFSSRAMGWFRQAPGKEREFVAVISWTGSPYYTDSVKGRFTISRDDAKNTVYLQMNSLKPEDTAVYYCAATSEHVMLVVTTRGGYDYWGQGTQVTVSS

SEQ ID NO: 8 - DNA, 375 bp, synthetic construct
  misc_feature 1..375: note = Description of Artificial Sequence: Synthetic polynucleotide
  source 1..375: mol_type = other DNA
caggtgcagctggcggagaccggcggaggatcggtgcaggctgggggctctctgacactctcctgtgcagcctctggacgcaccttcagtagcagagccatgggctggttccgccaggctccagggaaggagcgtgagtttgtagcagttattagctggactggtagcccatactatacagactccgtgaagggccgattcaccatctccagagacgacgccaagaacacggtgtatctgcaaatgaacagcctgaaacctgaggacacggccgtttattactgcgcagcgacgtcagaacatgtaatgctggtagttactacgcgtggcgggtatgactactggggccaggggacccaggtcaccgtctcctca

SEQ ID NO: 9 - protein, 131 aa, synthetic construct
  REGION 1..131: note = Description of Artificial Sequence: Synthetic polypeptide
QLQLVETGGGLVQPGGSLRLSCAASGFTLDYYAIGWFRQAPGKEREGVSCLSSSDRSTYYVDSVKGRFTISRDDDKNTAYLQMNSLKPEDTATYYCAAGTWDLKWGYNISACVGSYEYDYWGQGTQVTVSS

SEQ ID NO: 10 - DNA, 393 bp, synthetic construct
  misc_feature 1..393: note = Description of Artificial Sequence: Synthetic polynucleotide
  source 1..393: mol_type = other DNA
cagttgcagctggtggagacaggaggaggcttggtgcagcctggggggtctctgagactctcctgtgcagcctctggattcactttggattattatgccataggctggttccgccaggctccagggaaggagcgtgagggggtctcatgtttgagtagtagtgatcgtagcacatactatgtagactccgtgaagggccgattcaccatctccagagacgatgacaagaacacggcgtatctgcagatgaacagcctgaaacctgaggacacagccacttattactgtgcagcgggcacatgggatcttaaatggggctataacattagtgcctgcgtgggatcttatgagtatgactactggggccaggggacgcaggtcaccgtctcctca

SEQ ID NO: 11 - protein, 8 aa, synthetic construct
  REGION 1..8: note = Description of Artificial Sequence: Synthetic peptide
GFTLDYYA

SEQ ID NO: 12 - protein, 8 aa, synthetic construct
  REGION 1..8: note = Description of Artificial Sequence: Synthetic peptide
SSSDRSTY

SEQ ID NO: 13 - protein, 22 aa, synthetic construct
  REGION 1..22: note = Description of Artificial Sequence: Synthetic peptide
GTWDLKWGYNISACVGSYEYDY

SEQ ID NO: 14 - protein, 8 aa, synthetic construct
  REGION 1..8: note = Description of Artificial Sequence: Synthetic peptide
SSSDRSAY

SEQ ID NO: 15 - protein, 22 aa, synthetic construct
  REGION 1..22: note = Description of Artificial Sequence: Synthetic peptide
GTWDLKWGYNISACVRSYEYDY

SEQ ID NO: 16 - protein, 8 aa, synthetic construct
  REGION 1..8: note = Description of Artificial Sequence: Synthetic peptide
SSSDLKTY

SEQ ID NO: 17 - protein, 22 aa, synthetic construct
  REGION 1..22: note = Description of Artificial Sequence: Synthetic peptide
GTWDLKFGYNISACVGSYEYDY

SEQ ID NO: 18 - protein, 8 aa, synthetic construct
  REGION 1..8: note = Description of Artificial Sequence: Synthetic peptide
GFALDYYA

SEQ ID NO: 19 - protein, 8 aa, synthetic construct
  REGION 1..8: note = Description of Artificial Sequence: Synthetic peptide
GFTLDYYG

SEQ ID NO: 20 - protein, 22 aa, synthetic construct
  REGION 1..22: note = Description of Artificial Sequence: Synthetic peptide
GTWDLKFGYNITTCVRSSEYDY

SEQ ID NO: 21 - protein, 8 aa, synthetic construct
  REGION 1..8: note = Description of Artificial Sequence: Synthetic peptide
GFTSDYYG

SEQ ID NO: 22 - protein, 8 aa, synthetic construct
  REGION 1..8: note = Description of Artificial Sequence: Synthetic peptide
SSSDWSTY

SEQ ID NO: 23 - protein, 22 aa, synthetic construct
  REGION 1..22: note = Description of Artificial Sequence: Synthetic peptide
GTWDLKFGYNRSNCVRSAEYDY

SEQ ID NO: 24 - protein, 8 aa, synthetic construct
  REGION 1..8: note = Description of Artificial Sequence: Synthetic peptide
GFTLAYYG

SEQ ID NO: 25 - protein, 8 aa, synthetic construct
  REGION 1..8: note = Description of Artificial Sequence: Synthetic peptide
SSSDLSTY

SEQ ID NO: 26 - protein, 22 aa, synthetic construct
  REGION 1..22: note = Description of Artificial Sequence: Synthetic peptide
GTWDLKFGYSRSNCVRSYEYDY

SEQ ID NO: 27 - protein, 20 aa, synthetic construct
  REGION 1..20: note = Description of Artificial Sequence: Synthetic peptide
  MOD_RES 1: note = T or absent
  MOD_RES 2: note = S, T, or G
  MOD_RES 13: note = L or Q
  MOD_RES 14: note = R or G
XXGGGLVQPGGSXXLSCAAS

SEQ ID NO: 28 - protein, 18 aa, synthetic construct
  REGION 1..18: note = Description of Artificial Sequence: Synthetic peptide
  MOD_RES 1: note = I or V
  MOD_RES 15: note = V or I
  MOD_RES 16: note = S or A
  MOD_RES 18: note = L, I, or M
XGWFRQAPGKEREGXXCX

SEQ ID NO: 29 - protein, 38 aa, synthetic construct
  REGION 1..38: note = Description of Artificial Sequence: Synthetic polypeptide
  MOD_RES 1: note = V, I, T, or A
  MOD_RES 14: note = D, G, N, Y, or S
  MOD_RES 15: note = D or A
  MOD_RES 16: note = K or N
  MOD_RES 17: note = N, S, or D
  MOD_RES 18: note = T or A
  MOD_RES 19: note = A or V
  MOD_RES 20: note = Y or S
  MOD_RES 32: note = A or G
  MOD_RES 33: note = T or V
XDSVKGRFTISRDXXXXXXXLQMNSLKPEDTXXYYCAA

SEQ ID NO: 30 - protein, 11 aa, synthetic construct
  REGION 1..11: note = Description of Artificial Sequence: Synthetic peptide
  MOD_RES 1: note = W or R
  MOD_RES 2: note = G or D
XXQGTQVTVSS

SEQ ID NO: 31 - protein, 8 aa, synthetic construct
  REGION 1..8: note = Description of Artificial Sequence: Synthetic peptide
GRPFSSFA

SEQ ID NO: 32 - protein, 8 aa, synthetic construct
  REGION 1..8: note = Description of Artificial Sequence: Synthetic peptide
TWSRGTTH

SEQ ID NO: 33 - protein, 18 aa, synthetic construct
  REGION 1..18: note = Description of Artificial Sequence: Synthetic peptide
AAADGWKVVSTASPAYDY

SEQ ID NO: 34 - protein, 8 aa, synthetic construct
  REGION 1..8: note = Description of Artificial Sequence: Synthetic peptide
GRTFSSRA

SEQ ID NO: 35 - protein, 7 aa, synthetic construct
  REGION 1..7: note = Description of Artificial Sequence: Synthetic peptide
SWTGSPY

SEQ ID NO: 36 - protein, 19 aa, synthetic construct
  REGION 1..19: note = Description of Artificial Sequence: Synthetic peptide
AATSEHVMLVVTTRGGYDY

SEQ ID NO: 37 - protein, 25 aa, synthetic construct
  REGION 1..25: note = Description of Artificial Sequence: Synthetic peptide
  MOD_RES 2: note = L or V
  MOD_RES 5: note = A or V
  MOD_RES 7: note = T or S
  MOD_RES 11: note = L or S
  MOD_RES 14: note = P or A
  MOD_RES 16: note = G or D
  MOD_RES 19: note = R, T, or G
QXQLXEXGGGXVQXGXSLXLSCAAS

SEQ ID NO: 38 - protein, 18 aa, synthetic construct
  REGION 1..18: note = Description of Artificial Sequence: Synthetic peptide
  MOD_RES 1: note = V, M, or I
  MOD_RES 14: note = F or G
  MOD_RES 15: note = I or V
  MOD_RES 16: note = S or A
  MOD_RES 17: note = C, A, or V
  MOD_RES 18: note = I or L
XGWFRQAPGKEREXXXXX

SEQ ID NO: 39 - protein, 39 aa, synthetic construct
  REGION 1..39: note = Description of Artificial Sequence: Synthetic polypeptide
  MOD_RES 2: note = A, T, or V
  MOD_RES 13: note = R or G
  MOD_RES 15: note = Y, N, or D
  MOD_RES 16: note = A or D
  MOD_RES 18: note = S, N, or D
  MOD_RES 20: note = V or A
  MOD_RES 21: note = S, F, or Y
  MOD_RES 33: note = G or A
  MOD_RES 34: note = V or T
YXDSVKGRFTISXDXXKXTXXLQMNSLKPEDTXXYYCAA

SEQ ID NO: 40 - protein, 11 aa, synthetic construct
  REGION 1..11: note = Description of Artificial Sequence: Synthetic peptide
  MOD_RES 2: note = D or G
WXQGTQVTVSS

SEQ ID NO: 41 - protein, 10 aa, synthetic construct
  REGION 1..10: note = Description of Artificial Sequence: Synthetic peptide
DELGPRLMGK

SEQ ID NO: 42 - protein, 13 aa, synthetic construct
  REGION 1..13: note = Description of Artificial Sequence: Synthetic peptide
GAPVPYPDPLEPR

SEQ ID NO: 43 - protein, 5 aa, synthetic construct
  REGION 1..5: note = Description of Artificial Sequence: Synthetic peptide
GGGGS

SEQ ID NO: 44 - protein, 15 aa, synthetic construct
  REGION 1..15: note = Description of Artificial Sequence: Synthetic peptide
GGGGSGGGGSGGGGS

SEQ ID NO: 45 - protein, 34 aa, synthetic construct
  REGION 1..34: note = Description of Artificial Sequence: Synthetic polypeptide
EPKTPKPQGGGGSGGGGSGGGGSQGVQSQVQLVE

SEQ ID NO: 46 - protein, 8 aa, synthetic construct
  REGION 1..8: note = Description of Artificial Sequence: Synthetic peptide
EPKTPKPQ

SEQ ID NO: 47 - protein, 25 aa, synthetic construct
  REGION 1..25: note = Description of Artificial Sequence: Synthetic peptide
  MOD_RES 5: note = Q or V
  MOD_RES 11: note = L or S
  MOD_RES 14: note = A or P
QVQLXESGGGXVQXGGSLRLSCAAS

SEQ ID NO: 48 - protein, 14 aa, synthetic construct
  REGION 1..14: note = Description of Artificial Sequence: Synthetic peptide
  MOD_RES 2: note = F, Y, or V
  MOD_RES 9: note = E, C, or G
  MOD_RES 10: note = R or L
  MOD_RES 12: note = F, G, L, or W
  MOD_RES 14: note = A, S, or T
WXRQAPGKXXEXVX

SEQ ID NO: 49 - protein, 37 aa, synthetic construct
  REGION 1..37: note = Description of Artificial Sequence: Synthetic polypeptide
  MOD_RES 2: note = A, Q, T, or V
  MOD_RES 3: note = D or E
  MOD_RES 5: note = V or A
  MOD_RES 10: note = T or A
  MOD_RES 11: note = I or V
  MOD_RES 13: note = R or Q
  MOD_RES 15: note = N or K
  MOD_RES 17: note = K or A
  MOD_RES 20: note = V, L, or M
  MOD_RES 25: note = N or D
  MOD_RES 26: note = S or N
  MOD_RES 28: note = K or R
  MOD_RES 30: note = E or D
  MOD_RES 33: note = A or G
  MOD_RES 34: note = V, I, T, or M
YXXSXKGRFXXSXDXAXNTXYLQMXXLXPXDTXXYYC

SEQ ID NO: 50 - protein, 11 aa, synthetic construct
  REGION 1..11: note = Description of Artificial Sequence: Synthetic peptide
WGQGTQVTVSS

SEQ ID NO: 51 - protein, 8 aa, synthetic construct
  REGION 1..8: note = Description of Artificial Sequence: Synthetic peptide
DYKDDDDK

SEQ ID NO: 52 - protein, 10 aa, synthetic construct
  REGION 1..10: note = Description of Artificial Sequence: Synthetic His tag
  SITE 1..10: note = This sequence may encompass 5-10 residues
HHHHHHHHHH

SEQ ID NO: 53 - protein, 6 aa, synthetic construct
  REGION 1..6: note = Description of Artificial Sequence: Synthetic 6xHis tag
HHHHHH

SEQ ID NO: 54 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 55 - DNA, 1127 bp, Homo sapiens
  source 1..1127: mol_type = other DNA
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca

SEQ ID NO: 56 - protein, 211 aa, Mus musculus
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 57 - DNA, 1141 bp, Mus musculus
  source 1..1141: mol_type = other DNA
aaatatgagactggggatgtctgtagctcattctgctctggagcccaccaagaacgatagtcaattccagaaaccgctatgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacctagtgcgttatgcctaagcatatcagtttgtggacattcctcactgtggtcagaaaatatatcctgttgtcaggtatctgacttatgttgttctctacgaagaactgacaatatgaatgttgggacactattttaattatttttaatttattgataatttaaataagtaaactttaagttaatttatgattgatatttattatttttatgaagtgtcacttgaaatgttatatgttatagttttgaaatgataacctaaaaatctatttgatataaatattctgttacctagccagatggtttcttggaatgtataagtttacctcaatgaattgctaatttaaatatgtttttaaagaaatctttgtgatgtatttttataatgtttagactgtcttcaaacaaataaattatattatatttaaaaacc

SEQ ID NO: 58 - protein, 19 aa, synthetic construct
  REGION 1..19: note = Description of Artificial Sequence: Synthetic peptide
SGGGLVQPGGSLRLSCAAS

SEQ ID NO: 59 - protein, 19 aa, synthetic construct
  REGION 1..19: note = Description of Artificial Sequence: Synthetic peptide
TGGGLVQPGGSLRLSCAAS

SEQ ID NO: 60 - protein, 20 aa, synthetic construct
  REGION 1..20: note = Description of Artificial Sequence: Synthetic peptide
TGGGGLVQPGGSLRLSCAAS

SEQ ID NO: 61 - protein, 18 aa, synthetic construct
  REGION 1..18: note = Description of Artificial Sequence: Synthetic peptide
IGWFRQAPGKEREGVSCL

SEQ ID NO: 62 - protein, 18 aa, synthetic construct
  REGION 1..18: note = Description of Artificial Sequence: Synthetic peptide
VGWFRQAPGKEREGISCI

SEQ ID NO: 63 - protein, 18 aa, synthetic construct
  REGION 1..18: note = Description of Artificial Sequence: Synthetic peptide
IGWFRQAPGKEREGVSCM

SEQ ID NO: 64 - protein, 18 aa, synthetic construct
  REGION 1..18: note = Description of Artificial Sequence: Synthetic peptide
IGWFRQAPGKEREGVSCI

SEQ ID NO: 65 - protein, 18 aa, synthetic construct
  REGION 1..18: note = Description of Artificial Sequence: Synthetic peptide
IGWFRQAPGKEREGVACI

SEQ ID NO: 66 - protein, 39 aa, synthetic construct
  REGION 1..39: note = Description of Artificial Sequence: Synthetic polypeptide
YVDSVKGRFTISRDDDKNTAYLQMNSLKPEDTATYYCAA

SEQ ID NO: 67 - protein, 39 aa, synthetic construct
  REGION 1..39: note = Description of Artificial Sequence: Synthetic polypeptide
AIDSVKGRFTISRDGAKNTVYLQMNSLKPEDTAVYYCAA

SEQ ID NO: 68 - protein, 39 aa, synthetic construct
  REGION 1..39: note = Description of Artificial Sequence: Synthetic polypeptide
YTDSVKGRFTISRDNANNAVSLQMNSLKPEDTGVYYCAA

SEQ ID NO: 69 - protein, 39 aa, synthetic construct
  REGION 1..39: note = Description of Artificial Sequence: Synthetic polypeptide
YADSVKGRFTISRDYAKSTVSLQMNSLKPEDTGVYYCAA

SEQ ID NO: 70 - protein, 39 aa, synthetic construct
  REGION 1..39: note = Description of Artificial Sequence: Synthetic polypeptide
YADSVKGRFTISRDNAKSTVSLQMNSLKPEDTGVYYCAA

SEQ ID NO: 71 - protein, 39 aa, synthetic construct
  REGION 1..39: note = Description of Artificial Sequence: Synthetic polypeptide
YADSVKGRFTISRDSAKNTVYLQMNSLKPEDTGVYYCAA

SEQ ID NO: 72 - protein, 39 aa, synthetic construct
  REGION 1..39: note = Description of Artificial Sequence: Synthetic polypeptide
YADSVKGRFTISRDNAKNTVYLQMNSLKPEDTAVYYCAA

SEQ ID NO: 73 - protein, 39 aa, synthetic construct
  REGION 1..39: note = Description of Artificial Sequence: Synthetic polypeptide
YADSVKGRFTISRDNAKDTVYLQMNSLKPEDTAVYYCAA

SEQ ID NO: 74 - protein, 11 aa, synthetic construct
  REGION 1..11: note = Description of Artificial Sequence: Synthetic peptide
WDQGTQVTVSS

SEQ ID NO: 75 - protein, 11 aa, synthetic construct
  REGION 1..11: note = Description of Artificial Sequence: Synthetic peptide
RGQGTQVTVSS

SEQ ID NO: 76 - protein, 25 aa, synthetic construct
  REGION 1..25: note = Description of Artificial Sequence: Synthetic peptide
QLQLAETGGGLVQPGGSLRLSCAAS

SEQ ID NO: 77 - protein, 25 aa, synthetic construct
  REGION 1..25: note = Description of Artificial Sequence: Synthetic peptide
QVQLVESGGGLVQAGDSLTLSCAAS

SEQ ID NO: 78 - protein, 25 aa, synthetic construct
  REGION 1..25: note = Description of Artificial Sequence: Synthetic peptide
QLQLVESGGGLVQPGGSLGLSCAAS

SEQ ID NO: 79 - protein, 25 aa, synthetic construct
  REGION 1..25: note = Description of Artificial Sequence: Synthetic peptide
QVQLAETGGGSVQAGGSLTLSCAAS

SEQ ID NO: 80 - protein, 25 aa, synthetic construct
  REGION 1..25: note = Description of Artificial Sequence: Synthetic peptide
QLQLVETGGGLVQPGGSLRLSCAAS

SEQ ID NO: 81 - protein, 18 aa, synthetic construct
  REGION 1..18: note = Description of Artificial Sequence: Synthetic peptide
MGWFRQAPGKEREFVAAI

SEQ ID NO: 82 - protein, 18 aa, synthetic construct
  REGION 1..18: note = Description of Artificial Sequence: Synthetic peptide
MGWFRQAPGKEREFVAVI

SEQ ID NO: 83 - protein, 37 aa, synthetic construct
  REGION 1..37: note = Description of Artificial Sequence: Synthetic polypeptide
YADSVKGRFTISGDNAKNTVFLQMNSLKPEDTAVYYC

SEQ ID NO: 84 - protein, 37 aa, synthetic construct
  REGION 1..37: note = Description of Artificial Sequence: Synthetic polypeptide
YTDSVKGRFTISRDDAKNTVYLQMNSLKPEDTAVYYC

SEQ ID NO: 85 - protein, 150 aa, synthetic construct
  REGION 1..150: note = Description of Artificial Sequence: Synthetic polypeptide
  SITE 1..150: note = This sequence may encompass 1-30 "Gly Gly Gly Gly Ser" repeating units
  REGION 1..150: note = See specification as filed for detailed description of substitutions and preferred embodiments
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 86 - protein, 13 aa, synthetic construct
  REGION 1..13: note = Description of Artificial Sequence: Synthetic peptide
DICLPRWGCLWED

SEQ ID NO: 87 - protein, 277 aa, synthetic construct
  REGION 1..277: note = Description of Artificial Sequence: Synthetic polypeptide
QVQLVESGGGLVQPGGSLGLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVSSGGGGSGGGGSGGGGSQVQLVESGGGLVQPGGSLGLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVSS

SEQ ID NO: 88 - protein, 131 aa, synthetic construct
  REGION 1..131: note = Description of Artificial Sequence: Synthetic polypeptide
QVQLVESGGGLVQPGGSLGLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVSS

SEQ ID NO: 89 - protein, 296 aa, synthetic construct
  REGION 1..296: note = Description of Artificial Sequence: Synthetic polypeptide
MGWSCIILFLVATATGVHSQVQLVESGGGLVQPGGSLGLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVSSGGGGSGGGGSGGGGSQVQLVESGGGLVQPGGSLGLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVSS

SEQ ID NO: 90 - protein, 315 aa, synthetic construct
  REGION 1..315: note = Description of Artificial Sequence: Synthetic polypeptide
MGWSCIILFLVATATGVHSQVQLVESGGGLVQPGGSLGLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVSSGGGGSGGGGSGGGGSQVQLVESGGGLVQPGGSLGLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVSSHHHHHHDICLPRWGCLWED

SEQ ID NO: 91 - protein, 611 aa, synthetic construct
  REGION 1..611: note = Description of Artificial Sequence: Synthetic polypeptide
MGWSCIILFLVATATGVHSQVQLVESGGGLVQPGGSLGLSCAASGFTLAYYGIGWFRQAPGKEREGVACISSSDQVQLVESGGGLVQPGGSLGLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVLSTYYADSVKGRFTISRDNAKDTVYLQMNSLKPEDTAVYYCAAGTWDLKFGYSRSNCVRSYEYDYWGQGTQVTVSSGGGGSGGGGSGGGGSQVQLVESGGGLVQPGGSLGLSCAASGFTLAYYGIGWFRQAPGKEREGVACISSSDLSSSGGGGSGGGGSGGGGSQVQLVESGGGLVQPGGSLGLSCAASGFTLAYYGIGWFRQAPGKEREGVACISSSDLSTYYADSVKGTYYADSVKGRFTISRDNAKDTVYLQMNSLKPEDTAVYYCAAGTWDLKFGYSRSNCVRSYEYDYWGQGTQVTVSSRFTISRDNAKDTVYLQMNSLKPEDTAVYYCAAGTWDLKFGYSRSNCVRSYEYDYWGQGTQVTVSSHHHHHHHHHHHHDICLPRWGCLWEDDICLPRWGCLWED

SEQ ID NO: 92 - DNA, 1938 bp, synthetic construct
  misc_feature 1..1938: note = Description of Artificial Sequence: Synthetic polynucleotide
  source 1..1938: mol_type = other DNA
gaattcccccgccgccaccgccgccaccatgggctggtcctgcatcattctgtttctggtggccacagccaccggcgtgcactctcaagttcagctatgggctggtcctgcatcattctgtttctggtggccacagccaccggcgtgcactctcaagttcagctggttgagtctggcggcggactggttcaacctggtggatctctgggactgagctgtgccgcctctggctttacactggcctactatggttgagtctggcggcggactggttcaacctggtggatctctgggactgagctgtgccgcctctggctttacactggcctactatggcatcggctggttcagacaggcccctggcaaagagagagagggcgtcgcctgtatcagcagcagcgatctgagcacctactacgggcatcggctggttcagacaggcccctggcaaagagagagagggcgtcgcctgtatcagcagcagcgatctgagcacctactacgccgacagcgtgaagggcaccgacagcgtgaagggcagattcaccatcagccgggacaacgccaaggacaccgtgtacctgcagatgaacagcctgaagcctgagattcaccatcagccgggacaacgccaaggacaccgtgtacctgcagatgaacagcctgaagcctgaggacaccgccgtgtactattgtgccgccggaacctgggatctgaagttcggctacagccggtccaattgcgtgcggagctacgagggacaccgccgtgtactattgtgccgccggaacctgggatctgaagttcggctacagccggtccaattgcgtgcggagctacgagtacgattactggggccagggcacccaagtgacagtctcttctggcggaggcggatcaggtggcggaggaagcggaggcggcggaatacgattactggggccagggcacccaagtgacagtctcttctggcggaggcggatcaggtggcggaggaagcggaggcggcggaagtcaagtgcaactggtggagtcaagtgcaactggtggaatcaggcggaggccttgttcagcctggcggaagccttggactgtcttgtgctgccagcggattcacatcaggcggaggccttgttcagcctggcggaagccttggactgtcttgtgctgccagcggattcaccctggcctattacggaattggatggtttcggcaggccccagggaaagaacgagaaggggttgcctgcatcagctccagcgacctgcctggcctattacggaattggatggtttcggcaggccccagggaaagaacgagaaggggttgcctgcatcagctccagcgacctgtctacctattatgccgactccgtgaaaggccggtttaccatcagcagagataatgccaaagatacggtctatctccagatgaacttctacctattatgccgactccgtgaaaggccggtttaccatcagcagagataatgccaaagatacggtctatctccagatgaactctctcaagcccgaggatacactctcaagcccgaggatacagccgtctattactgcgccgctggcacatgggacctgaaatttggctactcccgcagcaactgcgtgccgtctattactgcgccgctggcacatgggacctgaaatttggctactcccgcagcaactgcgtgcggtcttacgagtatgactattggggacaagggacacaagtgaccgtgtccagccaccaccaccatcaccatgcggtcttacgagtatgactattggggacaagggacacaagtgaccgtgtccagccaccaccaccatcaccatgatatctgcctgatatctgcctgcctagatggggctgcctgtgggaagatgcctagatggggctgcctgtgggaagattgatgattaagcttaagctt

SEQ ID NO: 93 - protein, 125 aa, synthetic construct
  REGION 1..125: note = Description of Artificial Sequence: Synthetic polypeptide
SGGGLVQPGGSLRLSCAASGFTLDYYAIGWFRQAPGKEREGVSCLSSSDRSTYYVDSVKGRFTISRDDDKNTAYLQMNSLKPEDTATYYCAAGTWDLKWGYNISACVGSYEYDYWGQGTQVTVSS

SEQ ID NO: 94 - protein, 125 aa, synthetic construct
  REGION 1..125: note = Description of Artificial Sequence: Synthetic polypeptide
TGGGLVQPGGSLRLSCAASGFTLDYYAIGWFRQAPGKEREGVSCLSSSDRSTYYVDSVKGRFTISRDDDKNTAYLQMNSLKPEDTATYYCAAGTWDLKWGYNISACVGSYEYDYWGQGTQVTVSS

SEQ ID NO: 95 - protein, 125 aa, synthetic construct
  REGION 1..125: note = Description of Artificial Sequence: Synthetic polypeptide
SGGGLVQPGGSLRLSCAASGFTLDYYAIGWFRQAPGKEREGVSCLSSSDRSAYAIDSVKGRFTISRDGAKNTVYLQMNSLKPEDTAVYYCAAGTWDLKWGYNISACVRSYEYDYWGQGTQVTVSS

SEQ ID NO: 96 - protein, 125 aa, synthetic construct
  REGION 1..125: note = Description of Artificial Sequence: Synthetic polypeptide
SGGGLVQPGGSLRLSCAASGFTLDYYAVGWFRQAPGKEREGISCISSSDLKTYYTDSVKGRFTISRDNANNAVSLQMNSLKPEDTGVYYCAAGTWDLKFGYNISACVGSYEYDYWGQGTQVTVSS

SEQ ID NO: 97 - protein, 125 aa, synthetic construct
  REGION 1..125: note = Description of Artificial Sequence: Synthetic polypeptide
TGGGLVQPGGSLRLSCAASGFTLDYYAVGWFRQAPGKEREGISCISSSDLKTYYTDSVKGRFTISRDNANNAVSLQMNSLKPEDTGVYYCAAGTWDLKFGYNISACVGSYEYDYWGQGTQVTVSS

SEQ ID NO: 98 - protein, 125 aa, synthetic construct
  REGION 1..125: note = Description of Artificial Sequence: Synthetic polypeptide
TGGGLVQPGGSLRLSCAASGFTLDYYAVGWFRQAPGKEREGISCISSSDLKTYYADSVKGRFTISRDYAKSTVSLQMNSLKPEDTGVYYCAAGTWDLKFGYNISACVGSYEYDYWDQGTQVTVSS

SEQ ID NO: 99 - protein, 125 aa, synthetic construct
  REGION 1..125: note = Description of Artificial Sequence: Synthetic polypeptide
TGGGLVQPGGSLRLSCAASGFTLDYYAVGWFRQAPGKEREGISCISSSDLKTYYADSVKGRFTISRDYAKSTVSLQMNSLKPEDTGVYYCAAGTWDLKFGYNISACVGSYEYDYWGQGTQVTVSS

SEQ ID NO: 100 - protein, 125 aa, synthetic construct
  REGION 1..125: note = Description of Artificial Sequence: Synthetic polypeptide
SGGGLVQPGGSLRLSCAASGFTLDYYAVGWFRQAPGKEREGISCISSSDLKTYYADSVKGRFTISRDNAKSTVSLQMNSLKPEDTGVYYCAAGTWDLKFGYNISACVGSYEYDYWGQGTQVTVSS

SEQ ID NO: 101 - protein, 125 aa, synthetic construct
  REGION 1..125: note = Description of Artificial Sequence: Synthetic polypeptide
SGGGLVQPGGSLRLSCAASGFALDYYAVGWFRQAPGKEREGISCISSSDLKTYYADSVKGRFTISRDNAKSTVSLQMNSLKPEDTGVYYCAAGTWDLKFGYNISACVGSYEYDYWGQGTQVTVSS

SEQ ID NO: 102 - protein, 125 aa, synthetic construct
  REGION 1..125: note = Description of Artificial Sequence: Synthetic polypeptide
TGGGLVQPGGSLRLSCAASGFTLDYYGIGWFRQAPGKEREGVSCMSSSDLKTYYADSVKGRFTISRDSAKNTVYLQMNSLKPEDTGVYYCAAGTWDLKFGYNITTCVRSSEYDYRGQGTQVTVSS

SEQ ID NO: 103 - protein, 125 aa, synthetic construct
  REGION 1..125: note = Description of Artificial Sequence: Synthetic polypeptide
TGGGLVQPGGSLRLSCAASGFTSDYYGIGWFRQAPGKEREGVSCMSSSDLKTYYADSVKGRFTISRDSAKNTVYLQMNSLKPEDTGVYYCAAGTWDLKFGYNITTCVRSSEYDYRGQGTQVTVSS

SEQ ID NO: 104 - protein, 126 aa, synthetic construct
  REGION 1..126: note = Description of Artificial Sequence: Synthetic polypeptide
TGGGGLVQPGGSLRLSCAASGFTLDYYGIGWFRQAPGKEREGVSCISSSDWSTYYADSVKGRFTISRDNAKNTVYLQMNSLKPEDTAVYYCAAGTWDLKFGYNRSNCVRSAEYDYWGQGTQVTVSS

SEQ ID NO: 105 - protein, 125 aa, synthetic construct
  REGION 1..125: note = Description of Artificial Sequence: Synthetic polypeptide
SGGGLVQPGGSLRLSCAASGFTLAYYGIGWFRQAPGKEREGVACISSSDLSTYYADSVKGRFTISRDNAKDTVYLQMNSLKPEDTAVYYCAAGTWDLKFGYSRSNCVRSYEYDYWGQGTQVTVSS